SEQUENCE LISTING (37 sequences)

SEQ ID NO: 1 - protein, 24 aa, synthetic construct
KRVAPQKQMSIAKKKKVRRRRRRR

SEQ ID NO: 2 - protein, 17 aa, Homo sapiens
KRVAPQKQMSIAKKKKV

SEQ ID NO: 3 - protein, 25 aa, Simian immunodeficiency virus
KRTADGSEFESPKKKRKVRRRRRRR

SEQ ID NO: 4 - protein, 18 aa, Simian immunodeficiency virus
KRTADGSEFESPKKKRKV

SEQ ID NO: 5 - protein, 27 aa, synthetic construct
KRKLITSEEERSPAKRGRKSRRRRRRR

SEQ ID NO: 6 - protein, 20 aa, Homo sapiens
KRKLITSEEERSPAKRGRKS

SEQ ID NO: 7 - DNA, 20 bp, synthetic construct
  source 1..20: mol_type = other DNA
agcaaagaccccaacgagaa

SEQ ID NO: 8 - DNA, 17 bp, synthetic construct
  source 1..17: mol_type = other DNA
ggcggcggtcacgaact

SEQ ID NO: 9 - DNA, 20 bp, synthetic construct
  source 1..20: mol_type = other DNA
ctggttagttcttgccacgg

SEQ ID NO: 10 - DNA, 20 bp, synthetic construct
  source 1..20: mol_type = other DNA
gttgggcactgacaattccg

SEQ ID NO: 11 - DNA, 20 bp, synthetic construct
  source 1..20: mol_type = other DNA
aaggaaggtccgctggattg

SEQ ID NO: 12 - DNA, 20 bp, synthetic construct
  source 1..20: mol_type = other DNA
aaagtcccggaaaggagctg

SEQ ID NO: 13 - protein, 7 aa, Simian immunodeficiency virus
PKKKRKV

SEQ ID NO: 14 - DNA, 21 bp, Simian immunodeficiency virus
  source 1..21: mol_type = other DNA
cctaagaaaaagaggaaggtg

SEQ ID NO: 15 - protein, 10 aa, synthetic construct
PKKKRKVRRR

SEQ ID NO: 16 - DNA, 30 bp, synthetic construct
  source 1..30: mol_type = other DNA
cctaagaaaaagcgcaaggtgagaaggcgg

SEQ ID NO: 17 - protein, 12 aa, synthetic construct
PKKKRKVRRRRR

SEQ ID NO: 18 - DNA, 36 bp, synthetic construct
  source 1..36: mol_type = other DNA
cctaagaaaaagcgcaaggtgagaaggcggcggcgc

SEQ ID NO: 19 - protein, 14 aa, synthetic construct
PKKKRKVRRRRRRR

SEQ ID NO: 20 - DNA, 42 bp, synthetic construct
  source 1..42: mol_type = other DNA
cctaagaaaaagcgcaaggtgagaaggcggcggcgccgtcga

SEQ ID NO: 21 - protein, 16 aa, synthetic construct
PKKKRKVRRRRRRRRR

SEQ ID NO: 22 - DNA, 48 bp, synthetic construct
  source 1..48: mol_type = other DNA
cctaagaaaaagcgcaaggtgagaaggcggcggcgccgtcgaaggaga

SEQ ID NO: 23 - protein, 18 aa, synthetic construct
PKKKRKVRRRRRRRRRRR

SEQ ID NO: 24 - DNA, 54 bp, synthetic construct
  source 1..54: mol_type = other DNA
cctaagaaaaagcgcaaggtgagaaggcggcggcgccgtcgaaggagacgtcgc

SEQ ID NO: 25 - DNA, 54 bp, Simian immunodeficiency virus
  source 1..54: mol_type = other DNA
aaaagaaccgccgacggctccgaattcgagagccctaagaaaaagcgcaaggtg

SEQ ID NO: 26 - DNA, 81 bp, synthetic construct
  source 1..81: mol_type = other DNA
aaacgcaccgccgacggcagcgagtttgagtctcccaagaaaaagcgcaaagtagaaaggagaagaaggagaaggaggggg

SEQ ID NO: 27 - DNA, 51 bp, Homo sapiens
  source 1..51: mol_type = genomic DNA
aaaagggtggctccccagaagcagatgagcatagcaaagaaaaagaaggtg

SEQ ID NO: 28 - protein, 20 aa, synthetic construct
KRVAPQKQMSIAKKKKVRRR

SEQ ID NO: 29 - DNA, 60 bp, synthetic construct
  source 1..60: mol_type = other DNA
aaaagggtggctccccagaagcagatgagcatagcaaagaaaaagaaggtgagaaggcgg

SEQ ID NO: 30 - protein, 22 aa, synthetic construct
KRVAPQKQMSIAKKKKVRRRRR

SEQ ID NO: 31 - DNA, 65 bp, synthetic construct
  source 1..65: mol_type = other DNA
aaaagggtggctccccagaagcagatgagcatagcaaagaaaaagaaggtgagaaggcggcggcg

SEQ ID NO: 32 - DNA, 72 bp, synthetic construct
  source 1..72: mol_type = other DNA
aaaagggtggctccccagaagcagatgagcatagcaaagaaaaagaaggtgagaaggcggcggcgccgtcga

SEQ ID NO: 33 - protein, 26 aa, synthetic construct
KRVAPQKQMSIAKKKKVRRRRRRRRR

SEQ ID NO: 34 - DNA, 78 bp, synthetic construct
  source 1..78: mol_type = other DNA
aaaagggtggctccccagaagcagatgagcatagcaaagaaaaagaaggtgagaaggcggcggcgccgtcgaaggaga

SEQ ID NO: 35 - protein, 28 aa, synthetic construct
KRVAPQKQMSIAKKKKVRRRRRRRRRRR

SEQ ID NO: 36 - DNA, 84 bp, synthetic construct
  source 1..84: mol_type = other DNA
aaaagggtggctccccagaagcagatgagcatagcaaagaaaaagaaggtgagaaggcggcggcgccgtcgaaggagacgtcgc

SEQ ID NO: 37 - protein, 10 aa, Aequorea victoria
VAGHASGSPA

(19) United States
(12) Patent Application Publication
(10) Pub. No.: US 2026/0357296 A1
(43) Pub. Date: Nov. 12, 2026

(54) SYNTHETIC NUCLEAR LOCALIZATION SIGNALS AND METHODS OF THEIR USE

(71) Applicant: University of Washington, Seattle, WA (US)

(72) Inventors: Sam A. Golden, Seattle, WA (US); Eric Szelenyi, Seattle, WA (US)

(73) Assignee: University of Washington, Seattle, WA (US)

(21) Appl. No.: 19/484,804

(22) PCT Filed: May 15, 2024
(86) PCT No.: PCT/US2024/029476

§ 371 Date: Nov. 14, 2025

Related U.S. Application Data
(60) Provisional application No. 63/467,255, filed on May 17, 2023.
(60) Provisional application No. 63/601,131, filed on Nov. 20, 2023.

(51) Int. Cl. C12N 15/86

(52) U.S. Cl. CPC C12N 15/86; C12N 2750/14143

(57) ABSTRACT

Various implementations described herein relate to synthetic nuclear tags for delivering various agents to the nucleus of a cell. The synthetic nuclear tags include a polyarginine linker attached to a bipartite nuclear localization signal (NLS). According to some implementations, a detectable label linked to the synthetic nuclear tag is administered to a subject or a sample, and an imaging device is used to detect the detectable label. Particular implementations include a construct encoding the synthetic nuclear tag and an agent. An example method includes administering a therapeutic agent linked to the synthetic nuclear tag to a subject. Example methods and compositions described herein can be used for diagnostic, therapeutic, or research purposes.

### CROSS-REFERENCE TO RELATED APPLICATION(S)

This application is a U.S. National Stage application which claims priority to Internation Application No. PCT/US2024/029476, filed on May 15, 2024, which claims the priority of U.S. Provisional Patent Application No. 63/601,131, filed on Nov. 20, 2023, and U.S. Provisional Patent Application No. 63/467,255, filed on May 17, 2023, which are incorporated by reference herein in their entirety.

### REFERENCE TO SEQUENCE LISTING

The Sequence Listing associated with this application is provided in XML format in lieu of a paper copy and is hereby incorporated by reference into the specification. The name of the file containing the Sequence Listing is W149-0052US-Seq.xml. The file is 33,818 bytes, was created Nov. 14, 2025, and is being submitted electronically via Patent Center

### TECHNICAL FIELD

This application relates to compositions and methods for delivering agents (e.g., peptides, detectable labels, nucleic acids, etc.) to the nucleus of eukaryotic cells. It relates more specifically to non-naturally occurring nuclear localization tags and their uses, including to label cells for automatic analysis.

### BACKGROUND

Recent advancements in high-throughput volumetric fluorescent microscopy provide means to deconstruct whole-brain anatomy and function at cellular resolution. This cellular-to-whole-brain spatial integration allows for the mapping of a variety of structural-functional properties of the brain that is governed at the level of single cells, such as: cellular activity, cell-type distributions, endogenous genetic and exogenous viral infectivity patterns, and target engagement of psychoactive drugs.

### BRIEF DESCRIPTION OF THE DRAWINGS

FIG. 1 illustrates an example environment for delivery of an agent to a nucleus of a cell of a subject.

FIG. 2 illustrates an example process for delivering an agent to the nucleus of a cell.

FIG. 3 illustrates an example of a system for performing various functions described herein.

FIGS. 4A-4L illustrate the strategy and in vitro characterization of an arginine-rich NLS (ArgiNLS).

FIGS. 5A, 5B illustrate that poly-R7 synergizes bipartite SV40 NLS activity in vitro.

FIGS. 6A-6L illustrate optimized nuclear localization of fluorescence across major cortical cell-type classes in vivo.

FIGS. 7A, 7B provide representative images of viral expression from local barrel cortex co-injections. Example viral expression observed in a (7A) vGAT-Cre or (7B) vGlut1-Cre animal for Cre-dependent tdTomato and SV40nls-(top row) or ArgiNLS-EGFP (bottom row) viruses.

FIGS. 8A-8M illustrate that ArgiNLS-EGFP viral expression is not associated with physiological or behavioral deficits.

FIG. 9 illustrates representative systemic PHP.eB viral EGFP expression from behavioral cohort.

FIGS. 10A-10H illustrate enhanced performance of single-cell ML classification.

FIGS. 11A-11C illustrate representative tagged FP localization in brain and peripheral liver tissue of single animals infected with systemic Php.eB AAV.

FIGS. 12A-12D provide representative 2D raw images and performance of single-cell ML classification.

FIGS. 13A-13M illustrate enriched brain-wide segmentation of single-cells.

FIGS. 14A-14G illustrate validation of ML-based brain-wide segmentation of single-cells.

FIGS. 15A-15F illustrate optimized volumetric brain cell counting applications with ArgiNLS viral vectors.

FIGS. 16A, 16B illustrate cortical differences in single-cell labeling and segmentation.

FIG. 17 illustrates ArgiNLS-enriched subregions.

FIGS. 18A, 18B illustrate volumetric quantification of single-cells from systemic PHP.eB ArgiNLS-oScarlet labeling with 3 genetic payload amounts.

### DETAILED DESCRIPTION

The structure of the mammalian brain is complex and spans multiple spatial scales, requiring the development and application of spatially integrated methods to reveal key relationships with its function. High-throughput volumetric fluorescent microscopy pipelines, facilitated by light-sheet fluorescence microscopy (LSFM) (S. Corsetti, et al., J Neurosci Methods 319, 16-27 (2019)) and serial section block-face microscopy (T. Ragan, et al., Nat Methods 9, 255-258 (2012); A. Li, et al., Science 330, 1404-1408 (2010), have accelerated these efforts. Notably, they have democratized experimental access to spatially integrated intact whole-brain anatomy, at the resolution of single-cells and within computationally feasible datasets that are compatible with brain atlas mapping. For example, in combination with genetic fluorescent labeling, these techniques have facilitated the mapping of cellular activity (Y. Kim, et al., Front Neural Circuits 10, 3 (2016); Y. Kim, et al., Cell Rep 10, 292-305 (2015); H. Azevedo, et al., Neurobiol Stress 13, 100226 (2020); G. Cai, et al., Neurobiol Stress 20, 100478 (2022); A. V. Roland, et al., Biol Psychiatry (2023); R. Madangopal, et al., Biorxiv. [Preprint](2022); L. A. DeNardo, et al., Nat Neurosci 22, 460-469 (2019); D. R. Rijsketic, et al., Neuropsychopharmacology (2023); H. H. Hansen, et al., Mol Metab 47, 101171 (2021); N. Renier, et al. Cell 165(7):1789-1802 (2016); E. A. Susaki, et al., Cell 157, 726-739 (2014); H. Yamaguchi, et al., Biorxiv. [Preprint](2023); D. S. Roy, et al., Nat Commun 13, 1799 (2022)), cell-type distributions (Y. Kim, et al., Cell 171, 456-469.e22 (2017); S. Son, et al., J Neurosci 42, 5021-5033 (2022); M. Habart, et al., STAR Protoc 4, 101968 (2023); K. S. Matho, et al., Nature 598, 182-187 (2021)), and endogenous genetic (E. R. Szelenyi, et al., Cell Reports 43 (2024)) and connectivity patterns (Y. W. Wang, et al., Biorxiv. [Preprint](2023)).

However, while significant hardware-based advancements have been made in microscopy systems (T. Ragan, et al., Nat Methods 9, 255-258 (2012; E. H. K. Stelzer, et al., Nat Rev Methods Primers 1, 1-25 (2021); K. M. Dean, et al., Nat Protoc 17, 2025-2053 (2022)), and numerous software-based image analysis tools are readily available (Y. Kim, et al., Cell Rep 10, 292-305 (2015; N. Renier, et al. Cell 165(7):1789-1802 (2016); E. A. Susaki, et al., Cell 157, 726-739 (2014); F. Claudi, et al., eLife 10, e65751 (2021); C. J. Niedworok, et al., Nat Commun 7, 11879 (2016); A. L. Tyson, et al., PLOS Computational Biology 17, e1009074 (2021); A. L. Tyson, et al., Sci Rep 12, 867 (2022); M. Negwer, et al., Gigascience 12, giad035 (2022)), there has been a relative lack of genetic tool development that maximizes single-cell detection and quantification through the optimization of fluorescent signal. As opposed to hardware- and software-based approaches, genetic approaches may offer improved cellular detection without the prohibitively expensive acquisition of cutting-edge microscopy and computational equipment. Past genetic-based approaches have relied on fluorescent protein (FP) expression in its unmodified or modified forms (D. Choquet, et al., Nat Rev Neurosci 22, 237-255 (2021)), but the efficacy of these prior efforts have not been systematically examined at the level of single-cell discrimination across image datasets of various sizes. As a result, the quantitative accuracy of single-cell brain mapping measurements remains ambiguous both within and between studies.

Various implementations described herein relate to methods and compositions for nuclear localization of an agent. As used herein, “agent” and its equivalents refers to a peptide, a protein, a detectable label, a therapeutic agent, a vector, a nucleic acid, a chemical agent, or the like. In various implementations, the agent is linked to a synthetic nuclear tag configured to localize the agent to a cell of interest, such as the nucleus of the cell. In some implementations, the synthetic nuclear tag is configured to localize the agent to the nucleolus of the cell. Various implementations of the present disclosure include synthetic nuclear tags that include a nuclear localization signal (NLS) linked to a polyarginine linker. The NLS, in some implementations, is a classical monopartite or a classical bipartite NLS. In particular implementations, the NLS is a classical bipartite NLS. In various implementations, the polyarginine linker includes a polyarginine region, such as a region including 3, 4, 5, 6, 7, 8, 9, 10, 11, or 12 arginines. In particular implementations, the polyarginine linker includes at least 7 arginines. Various implementations of the present disclosure include an isolated agent linked to the synthetic nuclear tag. Various implementations include a nucleic acid encoding the synthetic nuclear tag. For instance, a nucleic acid encoding the synthetic nuclear tag and the agent, when expressed in cells, may target the agent to the nucleus or nucleolus of the cell.

Various implementations relate to methods localizing the agent to the nucleus of a cell. In some implementations, a method of delivering the agent to the cell nucleus includes contacting a cell with the agent linked to the synthetic nuclear tag. In various implementations, a method of delivering the agent to the cell nucleus includes transfecting a cell with a nucleic acid encoding the synthetic nuclear tag and the agent. In some implementations, a method of cell imaging includes introducing a detectable label linked to the synthetic nuclear tag to the cell, such as causing the cell to be in contact with the detectable label linked to the synthetic nuclear tag. Particular implementations include expressing, in a cell, the detectable label linked to the synthetic nuclear tag. Also described are methods of using this synthetic nuclear tag, for instance, for single cell labeling and 2-to-3D image segmentation through its ability to restrict signal from the attached detectable label near-exclusively in the nucleus.

Systems described herein include an imaging system. The imaging system, for instance, includes at least one sensor configured to detect a detectable label linked to the synthetic nuclear tag. In some cases, the sensor(s) generate an image that is analyzed by a processor. The processor, for instance, can perform one or more functionalities described herein to analyze the image. For example, the processor is configured to execute one or more instructions stored in memory or a non-transitory computer readable medium storing instructions for performing any functions described herein.

Various implementations of the present disclosure enable delivery of an agent to the nucleus of a cell without harming the health of the cell. Various implementations enable nuclear delivery to cells of an organism without harming the health of the organism. Various implementations of the present disclosure improve nuclear delivery. In various implementations, use of the described synthetic nuclear tags reduces off-target delivery and increases efficacy of nuclear delivery.

Particular examples are now described, with reference to the accompanying figures. The scope of this disclosure includes individual examples described herein as well as any combination of the examples and elements therein, unless otherwise specified.

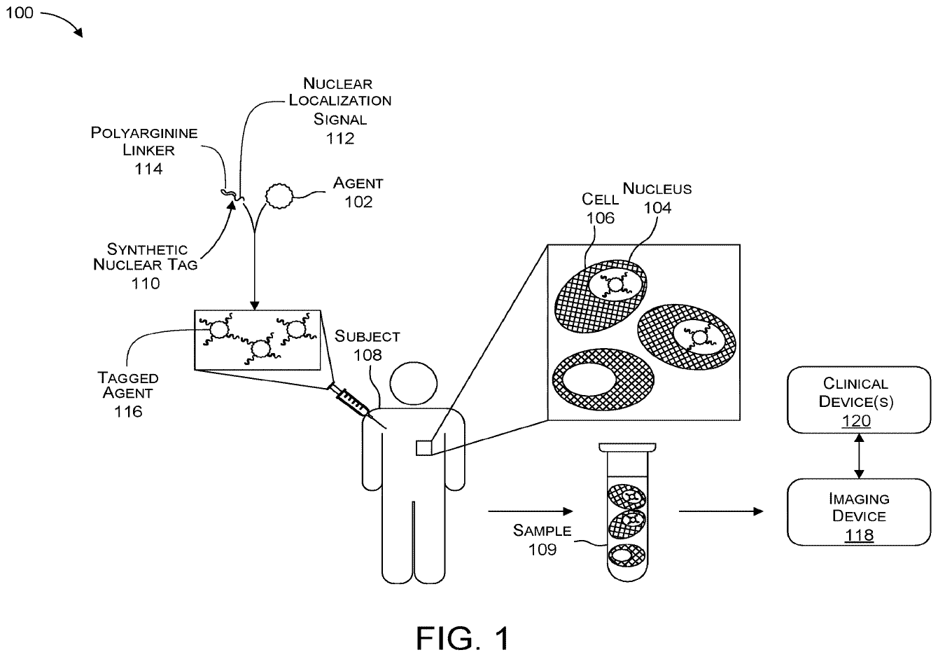

FIG. 1 illustrates an example environment 100 for delivery of an agent 102 to a nucleus 104 of a cell 106 of a subject 108. The agent 102, in various examples, includes a peptide, a protein, a detectable label, a therapeutic agent, a vector, a nucleic acid, a chemical agent, or the like. The subject 108, in some cases, may be suffering from a pathological condition, and the agent 102 may facilitate identification, diagnosis, or treatment of the pathological condition. The term “pathological condition,” and its equivalents, may refer to a disease, an abnormal anatomical condition, an abnormal physiological condition, a genetic disorder, a chronic illness, a short-term illness, a syndrome, a disorder, or the like. In some examples, the agent 102 may be administered to the subject 108 for research purposes.

In some implementations, the agent 102 is a detectable label. For instance, the agent 102 may be a fluorescent protein, a luminescent protein, a fluorophore, a quantum dot, a nanoparticle, a dye, a radioisotope, a contrast agent, or the like. Exemplary detectable labels include luminescent and/or fluorescent proteins, such as yellow fluorescent molecules such as SYFP2, Alexa Fluor 532, Citrine, PhiYFP, ZsYellow1, and Venus fluorescent protein (Nagai et al., Nature Biotech. 20:87-90, 2002); red fluorescent molecules such as Texas Red™, mCherry, mRuby, Jred, Alexa Fluor 594, AsRed2, and E2-Crimson; green fluorescent molecules such as green fluorescent protein (GFP), enhanced green fluorescent protein (EGFP), avGFP, ZsGreen, Alexa Fluor 488, mAzamiGreen, and FITC; orange fluorescent molecules such as Alexa Fluor 546, mOrange, and mKusabira-Orange; blue fluorescent molecules such as Sapphire, mKalama1, EBFP2, and Azurite; cyan fluorescent molecules such as Cerulean and mTurquoise; far red proteins such as mPlum and mNeptune; and cyanine fluorescent molecules such as Cy2, Cy3, Cy5, and Cy7. Other reporter proteins include AausFP1, mVenus-ME, and mGreenLantern. The fluorescent protein may be unmodified or modified. In some examples, the detectable label includes a contrast agent for detection using magnetic resonance imaging (MRI), computed tomography (CT), ultrasound, or the like. In some examples, the detectable label includes a radioisotope for detection using positron emission tomography (PET), single-photon emission computed tomography (SPECT), or the like.

In some examples, the agent 102 is a therapeutic agent. For example, the agent 102 may include a pharmaceutical agent, a biological agent, a chemical agent, gene therapy agent, an antiviral treatment (e.g., a nucleoside analog, an integrase inhibitor, etc.), a cancer treatment (e.g., a chemotherapy drug, a tyrosine kinase inhibitor, etc.), or another type of therapeutic agent. Gene therapy agents may include CRISPR/Cas9 system elements, zinc finger nucleases, plasmids, therapeutic genes, siRNAs, miRNAs, or the like. In some examples, a gene therapy agent may be encapsulated in a vector, such as a viral vector, a non-viral vector, any vector described herein, or any other vector known in the art. In some examples, the agent 102 is a nucleic acid configured to transform a cell. For instance, the agent 102 may be configured to modify the gene expression of one or more cell(s) of the subject. In some examples, the agent 102 is configured to modify a cell to express a detectable label.

While FIG. 1 illustrates the administration of the agent 102 to the subject 108, implementations of the present disclosure are not so limited. The agent 102, in various implementations, can be administered to a sample 109 derived from the subject 108. The subject 108, for instance, may be human, a non-human primate, a mouse, a rat, or another living organism. The sample 109 may include cells from a human, a non-human primate, a mouse, a rat, or another living organism. The sample 109 may include cells, fluid, tissue, or a combination thereof, collected from the subject 108. The sample 109 may include a brain cell, a central nervous system cell, a heart cell, an epithelial cell, an endothelial cell, a muscle cell, an immune cell, an adipocyte, an osteocyte, or a stem cell. In some examples, the brain cell is a glutamatergic cell or a gamma-aminobutyric acid(GABA)ergic cell. In some examples, the sample 109 includes modified cells, engineered cells, synthetic cells, chimeric cells, or the like. The sample 109 may include differentiated tissue or undifferentiated tissue. The agent, in some examples, can be administered in vivo or ex vivo.

In various implementations, it may be beneficial to deliver the agent 102 to the nucleus 104 of the cell 106 of the subject 108. Those of skill in the relevant art will understand myriad uses. Representative contemplated uses include nuclear delivery of the agent 102 that is intended to function within the nucleus 104 of the cell 106. For instance, it is beneficial to specifically deliver, to the nucleus 104, elements of a genome- or transcription-editing system (e.g., CRISPR/CAS9 system elements, elements of a light-inducible transcription-editing system, and the like). Moreover, specific delivery of a detectable label to the nucleus 104 can improve single cell labeling and image segmentation (e.g., 2D-to-3D image segmentation). In particular examples, visualization and segmentation of single brain cells from large-scale image volumes of complex tissue, such as that of the whole mouse brain, conventionally require genetically-encoded fluorescent labeling. Modified fluorescent proteins (FPs) that have reduced extranuclear expression can facilitate single cell discrimination and manual or machine segmentation during downstream image quantification.

Possible FP modifications used for this purpose include 1) short NLS tags (e.g., simian virus 40 (SV40nls)) or 2) full-length nuclear protein-FP fusions (e.g., H2B-FP) to increase the nuclear localization of the FP (Lanford & Butel, Cell37:801-813, 1984; Goertsen et al., Nat Neurosci 25:106-115, 2022; Jang et al. Nat Biotechnol 1-15, 2023; Kim et al., Cell 171:456-469.e22, 2017). However, some previously used NLS tag modifications for FP modifications, or for nuclear localization of other agents, sub-optimally achieve nuclear localization and are therefore unsuitable for precise large-scale volumetric image segmentation of single cells. The use of full-length nuclear protein fusions are nuclear-restrictive but cause robust detrimental effects to cellular, neuronal, and animal health (Ito et al., Nat Commun 5:4450, 2014).

Two major bottlenecks have slowed the development of new ectopic NLSs—including those identified in silico (M. Bernhofer, et al., Nucleic Acids Research 46, D503-D508 (2018)) or synthetic versions developed for genome-editing (K. Luk, et al., GEN Biotechnology 1, 271-284 (2022); T. Thumberger, et al., eLife 11 (2022))—for use as FP fusion tags. First, NLS performance when ectopically tagged as an FP fusion is unpredictable. This is because the nuclear import efficiency of naturally occurring NLSs are influenced by many factors, including intrinsic NLS sequence affinity to nuclear transport receptors KPNA and KPNB (B. Cautain, et al, FEBS J 282, 445-462 (2015)), and their interplay with the amino acids surrounding the NLS in its native state (B. Friedrich, et al., Mol Cell Biol 26, 8697-8709 (2006); C. Y. Xiao, et al., FEBS Lett 440, 297-301 (1998)). Second, due to the barrier posed by challenging biological properties, there is not a high-throughput pipeline to systematically screen novel NLSs for whole-brain mapping compatibility. Those properties include: (i) minimal extra-nuclear FP localization for high SNR, (ii) a simple and flexible sequence with minimal length and a single location of fused tag for unaltered FP function, (iii) modular compatibility with FP variants for spectral flexibility in imaging design, including multiplexing, (iv) equal functionality across cell-types for unbiased labeling of single-cells, and (v) non-deleterious effects of cellular and animal health. Thus, a genetic tag strategy that simultaneously synergizes and stereotypes FP-tagged NLS function while maintaining cellular health could satisfy these requirements.

These issues can be addressed, for instance, by using a non-deleterious synthetic nuclear tag 110 that does not harm, or otherwise affect, the health of the subject 108 and provides improved localization to the nucleus 104 compared to various minimal NLSs. In various implementations, administration of the synthetic nuclear tag 110 to the subject 108 or to the sample 109 does not cause deleterious effects to the cellular health or animal health (e.g., behavioral health) of the subject 108 or the sample 109. In some implementations, the synthetic nuclear tag 110 localizes the agent 102 to the nucleus 104 without significant off-target (e.g., extranuclear) localization. In various cases, the synthetic nuclear tag 110 localizes the agent 102 to the nucleolus of the cell 106 without significant off-target localization. For instance, the synthetic nuclear tag 110 may be linked to a detectable label and applied to a tissue sample, allowing for cell-by-cell analysis of the tissue, such as brain tissue, based on detection of cellular nuclei. Because of the low non-targeted localization, the synthetic nuclear tag 110 may enhance computer-assisted analysis of tissue labeled with, for instance, a fluorescent protein targeted to the nucleus 104 by the synthetic nuclear tag 110. In general, various implementations described herein can be used to enhance processes that are based in localizing agent(s) 102 to the nucleus 104 for control over visualization or any biological function of the cell 106 that is governed by or within the nucleus 104.

The synthetic nuclear tag 110 also can be used to improve other methods systems that rely on labeled nuclei—for instance, systems or methods that involve isolation of nuclei. The high-fidelity nuclear labelling enabled using the synthetic nuclear tag 110, and the very low non-nuclear background, permit improved separation of nuclei from non-nuclear material of cells. For instance, droplet-based single-nucleus genomics (e.g., single cell sequencing) can be improved by delivering, to the target nuclei, a detectable label using the synthetic nuclear tag 110. Various example techniques for single-nucleus sequencing are described, for instance, in Alvarez et al. (Sci Rep. 10, Art. No. 11019, 2020), Hahn et al. (NAR 49(2): e11, 2021), Van Hauwaert et al. (STAR Protocols, 2(3):100612, 2021).

The synthetic nuclear tag 110, in various implementations, includes a nuclear localization signal (NLS) 112 linked to a polyarginine linker 114. In some examples, the NLS 112 is a classical NLS or a non-classical NLS. The classical NLS, in various cases, may include one stretch of basic amino acids (e.g., a monopartite NLS) or two stretches of basic amino acids separated by a linker (e.g., a bipartite NLS). In various implementations of the present disclosure, the NLS 112 is a classical bipartite NLS. For instance, the NLS 112 may include SV40nls, nucleoplasmin 2 (NPM2) NLS, or another bipartite NLS (including fully synthetic bipartite NLSs as well as those known in the art; see, e.g., Lange et al., Traffic 11(3):311-323, 2010; Lu et al., Cell Commun. Signal. 19, Art. No. 60, 2021). In various implementations, the bipartite NLS includes the sequence set forth in (K/R)-X₁₀₋₁₂-(K/R/X), where K/R represents any combination of any length of K and R, X represents any amino acid, and K/R/X represents any combination of any length of K, R, and X. In various implementations, the monopartite NLS includes the sequence set forth in (K/R)-X-(K/R). The polyarginine linker 114 may include 3, 4, 5, 6, 7, 8, 9, 10, 11, or 12 arginines. In some examples, the polyarginine linker 114 includes at least 7 arginines. In particular examples, the polyarginine linker 114 includes 7 arginines.

A particular implementation of the synthetic nuclear tag 110, referred to herein as “ArgiNLS” (arginine-NLS) has the sequence KRVAPQKQMSIAKKKKVRRRRRRR (SEQ ID NO: 1); this sequence includes a bipartite NLS (e.g., the NLS 112) at positions 1-17 (with basic domains at positions 1-3 and 13-17), having the sequence KRVAPQKQMSIAKKKKV (SEQ ID NO: 2) and the polyarginine linker 114 at positions 18-24. Another implementation of the synthetic nuclear tag 110 has the sequence KRTADGSEFESPKKKRKVRRRRRRR (SEQ ID NO: 3); this sequence includes a bipartite NLS (e.g., the NLS 112) derived from SV40 at positions 1-17, having the sequence KRTADGSEFESPKKKRKV (SEQ ID NO: 4). Another implementation of the synthetic nuclear tag 110 has the sequence KRKLITSEEERSPAKRGRKSRRRRRRR (SEQ ID NO: 5); this sequence includes a bipartite NLS (e.g., the NLS 112) derived from Tp53-binding protein 1 (53BP1) at positions 1-20, having the sequence KRKLITSEEERSPAKRGRKS (SEQ ID NO: 6).

The synthetic nuclear tag 110 may be linked to the agent 102 to produce a tagged agent 116 according to various techniques known in the art. For instance, the synthetic nuclear tag 110 may be linked to the agent 102 via crosslinking, click chemistry, disulfide bond formation, carbodiimide coupling, enzymatic conjugation, thiol-reactive chemistry, amine-reactive chemistry, biotinylation, passive conjugation, lipidation, genetic fusion, or any other suitable technique. In some examples, the agent 102 is a protein or a peptide, and the synthetic nuclear tag 110 may be at the N or the C terminus of the protein or the peptide. In some examples, the synthetic nuclear tag 110 is near the N or the C terminus of the protein or the peptide. In some instances, N-terminal attachment may be preferred than C-terminal attachment, due to ease, efficacy, or stability of the conjugation or performance of the tagged agent 116; in others, C-terminal attachment may be preferred.

In various implementations, administering the tagged agent 116 does not cause deleterious effects to the health of the subject 108, even with systemic expression. The non-deleterious and segmentation-optimized labeling provided by the synthetic nuclear tag 110 permits both short and long-term study of unaltered cellular function of single cells. This characteristic enables use of the synthetic nuclear tag 110 in a variety of experimental settings where optimal cell counting and cellular health is required, such as but not limited to: behavioral experimentation, optical and electrophysiological recordings, transcriptomic and proteomic cellular profiling, anatomical tracing studies including transsynaptic antero- or retro-grade labeling conventions, and the study of disease models.

For instance, based on administering the tagged agent 116 to the subject 108, the health of the subject 108 may be evaluated. For instance, a behavior of the subject 108, a physiological parameter of the subject 108, a marker of cellular health, or another metric of the health of the subject 108 may be evaluated. In some examples, behavioral health may include locomotion tests, an open field test, an elevated plus maze test, a Morris water maze, a novel object recognition test, a forced swim test, a tail suspension test, a social interaction test, a maze test, an operant conditioning test, a self-report assessment, a clinical interview, a psychological assessment, an observational test, a functional assessment, or another behavioral assessment known in the art. In some examples, a physiological parameter may include a blood marker, a urine marker, a vital sign, or the like. In some examples, a marker of cellular health may include cell viability, cellular proliferation, metabolic activity, oxidative stress, DNA integrity, DNA damage, cellular morphology, cellular structure, cellular signaling, cellular function, or the like. In various examples, the health of the subject 108 may not be impacted within 30 minutes, 1 hour, 6 hours, 12 hours, 1 day, 2 days, 3 days, 4 days, 5 days, 6 days, 1 week, 2 weeks, 3 weeks, 1 month, 2 months, 3 months, 6 months, 9 months, 1 year, or more than one year from administration of the tagged agent 116.

In some examples, a nucleic acid encoding the nuclear tag (e.g., an expression construct) may be administered to the subject 108 or the sample 109. The expression construct is, in various cases, configured to transform the cell 106 to express the synthetic nuclear tag 110. The expression construct may be within a vector. In some examples, the expression construct may encode the synthetic nuclear tag 110 and a protein (e.g., the agent 102) to be localized to the nucleus 104. The expression construct may encode the synthetic nuclear tag 110 to be at the C-terminus or the N-terminus of the protein. In some examples, the synthetic nuclear tag 110 may be embedded at any position within an open reading frame of the expression construct. The expression construct may be administered to the subject 108 or the sample 109.

In addition to nucleic acid(s) encoding the synthetic nuclear tag 110 and encoding the protein to be targeted to the nucleus, one of skill in the art will recognize that expression constructs for use herein may have additional components, including elements required for expression, replication, and/or maintenance in the host cell. Such elements may be useful across multiple platforms or may be specific for the host cell type or other aspects. The following provides representative elements, though others will be known and recognized by those of skill in the art.

Animal cells in particular can be transformed using viral vectors; generally, this phrase refers to a nucleic acid molecule that includes virus-derived nucleic acid elements that facilitate transfer and expression of non-native nucleic acid molecules within a cell. Adeno-associated viral vector are viral vectors or plasmids containing structural and functional genetic elements, or portions thereof, that are primarily derived from AAV. Retroviral vectors are viral vectors or plasmids containing structural and functional genetic elements, or portions thereof, that are primarily derived from a retrovirus. Lentiviral vectors are viral vectors or plasmids containing structural and functional genetic elements, or portions thereof, that are primarily derived from a lentivirus, and so on. Hybrid vector are vectors including structural and/or functional genetic elements from more than one virus type.

Adenovirus vectors are constructs containing adenovirus sequences sufficient to (a) support packaging of the expression construct and (b) to express a coding sequence that has been cloned therein in a sense or antisense orientation. A recombinant Adenovirus vector includes a genetically engineered form of an adenovirus. Knowledge of the genetic organization of adenovirus, a 36 kb, linear, double-stranded DNA virus, allows substitution of large pieces of adenoviral DNA with foreign sequences up to 7 kb. In contrast to retrovirus, the adenoviral infection of host cells does not result in chromosomal integration because adenoviral DNA can replicate in an episomal manner without potential genotoxicity. Also, adenoviruses are structurally stable, and no genome rearrangement has been detected after extensive amplification.

Adenovirus can be particularly suitable for use as a gene transfer vector because of its mid-sized genome, ease of manipulation, high titer, wide target-cell range, and high infectivity. Both ends of the viral genome contain 100-200 base pair inverted repeats (ITRs), which are cis elements necessary for viral DNA replication and packaging. The early (E) and late (L) regions of the genome contain different transcription units that are divided by the onset of viral DNA replication. The E1 region (E1A and E1B) encodes proteins responsible for the regulation of transcription of the viral genome and a few cellular genes. The expression of the E2 region (E2A and E2B) results in the synthesis of the proteins for viral DNA replication. These proteins are involved in DNA replication, late gene expression and host cell shut-off. The products of the late genes, including the majority of the viral capsid proteins, are expressed only after significant processing of a single primary transcript issued by the major late promoter (MLP). The MLP is particularly efficient during the late phase of infection, and all the mRNAs issued from this promoter possess a 5-tripartite leader (TPL) sequence which makes them preferred mRNAs for translation.

The typical vector for animal cell transformation is replication defective and will not have an adenovirus E1 region. Thus, it will be most convenient to introduce the polynucleotide encoding the gene of interest at the position from which the E1-coding sequences have been removed. However, the position of insertion of the expression construct within the adenovirus sequences is not critical. The polynucleotide encoding the gene of interest may also be inserted in lieu of a deleted E3 region in E3 replacement vectors or in the E4 region where a helper cell line or helper virus complements the E4 defect.

Adeno-Associated Virus (AAV) is a parvovirus, discovered as a contamination of adenoviral stocks. It is a ubiquitous virus that has not been linked to any disease. AAV is also classified as a dependovirus because its replication is dependent on the presence of a helper virus, such as adenovirus. Various serotypes have been isolated, of which AAV-2 is the best characterized. AAV has a single-stranded linear DNA that is encapsidated into capsid proteins VP1, VP2 and VP3 to form an icosahedral virion of 20 to 24 nm in diameter.

The AAV DNA is 4.7 kilobases long. It contains two open reading frames and is flanked by two ITRs. There are two major genes in the AAV genome: rep and cap. The rep gene codes for proteins responsible for viral replications, whereas cap codes for capsid protein VP1-3. Each ITR forms a T-shaped hairpin structure. These terminal repeats are the only essential cis components of the AAV for chromosomal integration. Therefore, the AAV can be used as a vector with all viral coding sequences removed and replaced by the cassette of genes for delivery. Three AAV viral promoters have been identified and named p5, p19, and p40, according to their map position. Transcription from p5 and p19 results in production of rep proteins, and transcription from p40 produces the capsid proteins.

Other viral vectors may also be employed. For example, vectors derived from viruses such as vaccinia virus, polioviruses and herpes viruses may be employed. They offer several attractive features for various mammalian cells.

Retroviruses are a common tool for gene delivery. “Retrovirus” refers to an RNA virus that reverse transcribes its genomic RNA into a linear double-stranded DNA copy and subsequently covalently integrates its genomic DNA into a host genome. Once the virus is integrated into the host genome, it is referred to as a “provirus.” The provirus serves as a template for RNA polymerase II and directs the expression of RNA molecules which encode the structural proteins and enzymes needed to produce new viral particles.

Illustrative retroviruses suitable for use in particular implementations, include: Moloney murine leukemia virus (M-MuLV), Moloney murine sarcoma virus (MoMSV), Harvey murine sarcoma virus (HaMuSV), murine mammary tumor virus (MuMTV), gibbon ape leukemia virus (GaLV), feline leukemia virus (FLV), spumavirus, Friend murine leukemia virus, Murine Stem Cell Virus (MSCV) and Rous Sarcoma Virus (RSV)) and lentivirus.

“Lentivirus” refers to a group (or genus) of complex retroviruses. Illustrative lentiviruses include: HIV (human immunodeficiency virus; including HIV type 1, and HIV type 2), Visna Maedi virus (VMV), the caprine arthritis-encephalitis virus (CAEV), equine infectious anemia virus (EIAV), feline immunodeficiency virus (FIV), bovine immune deficiency virus (BIV), and simian immunodeficiency virus (SIV). In particular implementations, HIV based vector backbones (e.g., HIV cis-acting sequence elements) can be used.

Elements directing the efficient termination and polyadenylation of a heterologous nucleic acid transcript can increase heterologous gene expression. Transcription termination signals are generally found downstream of the polyadenylation signal. In particular implementations, vectors include a polyadenylation sequence 3′ of a polynucleotide encoding a polypeptide to be expressed. The term “poly(A) site” or “poly(A) sequence” denotes a DNA sequence which directs both the termination and polyadenylation of the nascent RNA transcript by RNA polymerase II. Polyadenylation sequences can promote mRNA stability by addition of a poly(A) tail to the 3′ end of the coding sequence and thus, contribute to increased translational efficiency. Particular implementations may utilize BGHpA or SV40 pA. In particular implementations, a preferred implementation of an expression construct includes a terminator element. These elements can serve to enhance transcript levels and to minimize read through from the construct into other plasmid sequences.

Beyond the foregoing description, a wide range of suitable expression vector types will be known to a person of ordinary skill in the art. These can include commercially available expression vectors designed for general recombinant procedures, for example plasmids that contain one or more reporter genes and regulatory elements required for expression of the reporter gene in cells. Numerous vectors are commercially available, e.g., from Invitrogen (of Waltham, MA), Stratagene (of La Jolla, CA), Clontech (of Mountain View, CA), etc., and are described in numerous associated guides. In particular implementations, suitable expression vectors include any plasmid, cosmid or phage construct that is capable of supporting expression of encoded genes in mammalian cell, such as pUC or Bluescript™ (Stratagene) plasmid series.

The nucleic acids described herein (including specifically expression constructs, such as vectors or plasmids, that include a sequence encoding the synthetic nuclear tag 110) can be introduced into the cell 106 by techniques known in the art. Such techniques may be tailored to be better suited to the specific cell type into which the nucleic acid(s) are being introduced. The phrase “introducing a nucleic acid into a cell” includes any method for introducing an exogenous nucleic acid molecule into a selected host cell (e.g., the cell 106), including transformation, transfection, and transduction. Examples of such methods include calcium phosphate- or calcium chloride-mediated transfection, electroporation, microinjection, particle bombardment, liposome-mediated transfection, transfection using bacterial bacteriophages, transduction using retroviruses or other viruses (such as vaccinia virus or baculovirus of insect cells), cell fusion, chromosome-mediated gene transfer, microcell-mediated gene transfer, spheroplast fusion, cell penetrating peptides, or other methods.

Liposome-mediated delivery methods are approaches using liposomes such as cationic liposomes, for example, cholesterol-based cationic liposomes. The method of using liposomes also includes lipofection, which utilizes the anionic electric properties of the cell surface. Alternatively, liposomes having surface bound with a cell membrane-permeable peptide (e.g., HIV-1 Tat peptide, penetratin, and oligoarginine peptide) can be used.

In particular implementations, the nucleic acids described herein are stably integrated into the genome of the cell 106. In particular implementations, the nucleic acids are stably maintained in the cell 106 as a separate, episomal segment. Transposons and transposable elements can be used to improve the efficiency of integration, the size of the DNA sequence integrated, and the number of copies of a DNA sequence integrated into a genome. Transposons or transposable elements include a short nucleic acid sequence with terminal repeat sequences upstream and downstream. Active transposons can encode enzymes that facilitate the excision and insertion of nucleic acid into a target DNA sequence.

In particular implementations, the nucleic acids can incorporate chemical groups that alter the physical characteristics of the nucleic acid and reduce degradation in the cell 106. As an example, the internucleotide phosphate ester can be optionally substituted with sulfur.

In particular implementations, nucleic acid constructs or other implementations described herein can be delivered using cell penetrating peptides. CPPs are short peptides that facilitate cellular uptake of various molecular cargo (from nanosize particles to small chemical molecules and large fragments of DNA). The “cargo” is associated with the peptides either through chemical linkage via covalent bonds or through non-covalent interactions. CPPs are of different sizes, amino acid sequences, and charges but all CPPs have one distinct characteristic: the ability to translocate the plasma membrane and facilitate the delivery of various molecular cargoes intracellularly. CPPs may enter cells through, for example, direct penetration of the membrane, endocytosis-mediated entry, or translocation through the formation of a transitory structure. Examples of CPPs include a transportan peptide (TP), a TP10 peptide, a pVEC peptide, a penetratin peptide, a tat fragment peptide, a signal sequence-based peptide, and an amphiphilic model peptide.

Particular implementations of the present disclosure can be delivered without cell penetrating peptides. The polyarginine linker 114, in some examples, enables entry of the tagged agent 116 into the cell 106. In some examples, cellular entry may be improved by use of the polyarginine linker 114 that includes nine arginines (Herce et al., Biophys J97(7):1917-25, 2009).

Constructs that express the tagged agent 116 can be introduced into the cell 106 using any conventional methods. Similarly, the construct can be conditional, in which a second protein is required to regulate its expression such as recombinases (e.g., Cre, Flp, Dre recombinases) or chemically-regulated transcription modifying proteins (e.g., TetR repressor protein). In addition, the construct can be gene-targeting for the creation of genetically-modified animals that genomically encode the tagged agent 116. The latter constructs can be used to create stereotyped expression of the tagged agent 116 based on genomic positioning in DNA-modified organisms and can be used in combination with a viral DNA-encoded tagged agent.

Particular implementations of the present disclosure include kits. For instance, a kit may include two or more spectrally separated detectable labels (e.g., fluorescent proteins). In some examples, one or more of the fluorescent proteins may be linked to the synthetic nuclear tag 110. Such kits (and host cells (e.g., the cell 106) or host animals (e.g., the subject 108) to which they are administered) are useful to label and analyze cells using different, distinguishable nuclear-localized detectable labels. The detectable labels can include any recombinant or naturally occurring peptide/protein that emit light of any wavelength upon excitation and can be combined in any manner. For instance, the synthetic nuclear tag 110 may be linked to a first detectable label (e.g., the agent 102), and a drug may be linked to a second detectable label. The tagged agent 102 and the tagged drug may be administered to the subject 108 or to a sample 109, and the distribution of the first detectable label and the second detectable label may be compared to determine the nuclear localization or cellular distribution of the drug.

In some implementations that involve detection of a nucleus labeled with a detectable label (e.g., the agent 102), the detectable label may be linked to the synthetic nuclear tag 110. In some implementations, the detectable label (e.g., a protein) is encoded by a reporter gene that is linked to the nuclear synthetic tag 110 or to a nucleic acid encoding the nuclear synthetic tag 110. Any reporter genes and/or detectable labels known in the art can be delivered to nuclei using the synthetic nuclear tag(s) 110 provided herein. It is particularly contemplated that the reporter gene enables detection, measurement, distinction, and/or separation of cells based on the presence or amount of the corresponding detectable label. In some implementations, a reporter gene encodes a detectable label that can readily be measured. For instance, an activity of the protein can be measured. In various implementations, the detectable label provides a low off-target (e.g., extranuclear) signal. Specific examples of the reporter gene may include a luminescent enzyme gene, a fluorescent protein gene, or the like. The detectable label (and the reporter gene encoding it) can be selected at least in part based on a device to be used for detecting the resultant signal, such as an activity of the detectable label.

There are myriad art-recognized methods for detecting detectable labels such as those discussed above. By way of example, detectable labels can be detected using a transcription-based assay, flow cytometry, Western blot assay, microscopy, fluorescence assay, luminescence assay, or the like. Detection system(s) are generally tailored for the detectable label being measured.

In some implementations, an imaging device 118 includes a sensor configured to detect the detectable label in the subject 108 or in the sample 109. For instance, the imaging device 118 may include a charge-coupled device (CCD) camera, a complementary metal-oxide-semiconductor (CMOS) camera, a scientific CMOS camera, a photomultiplier tube (PMT), a computed tomography (CT) detector, a magnetic resonance imaging (MRI) detector, a digital radiography (DR) detector, a gamma camera (e.g., a nuclear camera), or another detector known in the art. The imaging device 118, in various cases, may include a light source and an optical system. The light source may be configured to emit an excitation light. The excitation light may illuminate a chamber containing the sample 109. The light source may include at least one of a xenon arc lamp, a mercury-vapor lamp, a light emitting diode (LED), a laser, or a combination thereof. In some cases, the light source is tunable, such that the wavelength and/or spectrum of the excitation light can be adjusted. In various implementations, the imaging device 118 is configured to communicate with a clinical device 120. For instance, the imaging device 118 may be configured to transmit, to the clinical device 120, data indicative of the signals detected by the sensor.

In some examples, the imaging device 118 is configured to perform additional functions. For instance, the imaging device 118 may be configured to manipulate the sample 109 based on detecting the detectable label. In some examples, the imaging device 118 is a flow cytometer. The imaging device 118 may be configured to detect a fluorescent signal emitted by the agent 102. In some examples, the imaging device 118 is configured to sort one or more cell(s) 106 based on the fluorescent signal. For instance, the imaging device 118 may be configured to perform fluorescence-activated cell sorting (FACS). In various cases, the imaging device 118 is configured to amplify the fluorescent signal using one or more techniques. In some examples, the imaging device 118 is configured to detect one or more properties of the cell 106 (e.g., forward light scatter, side light scatter, fluorescence polarization, intracellular proteins, cell surface markers, DNA content, and the like).

The clinical device 120, in various examples, is configured to analyze the signals detected by the sensor. For instance, the clinical device 120 may be a computing device that includes one or more processor(s). Examples of computing devices include server computers, desktop computers, laptop computers, tablet computers, mobile phones, wearable devices, Internet of Things (IoT) devices, and the like. In various cases, instructions for performing at least a portion of the functions of these elements are stored in memory and/or in a non-transitory computer readable medium. The instructions, for instance, are executed by the processor(s).

The clinical device 120 may be configured to analyze the signals detected by the sensor. For instance, the clinical device 120 may be configured to apply a segmentation technique (e.g., a 2D segmentation technique, a 2D to 3D segmentation technique, etc.), a machine learning technique, a feature extraction technique, an image analysis technique, or another technique. In some examples, the imaging device 118 or clinical device 120 may be configured to amplify the fluorescent signal using one or more techniques. For example, the imaging device 118 may include a photomultiplier tube, a semiconductor diode, or the like. The imaging device 118, in some examples, utilizes a technique such as stimulated emission depletion (STED) microscopy, stochastic optical reconstruction microscopy (STORM), structured illumination microscopy (SIM), or the like. In various cases, the clinical device 120 is configured to apply a filtering technique to reduce background signal and amplify target signal, such as band-pass filtering, spatial filtering, deconvolution, or the like.

For instance, the imaging device 118 may include at least one photodetector. In some cases, the imaging device 118 includes multiple sensors arranged in an array. In various implementations, the sensor(s) within the imaging device 118 can sense, and differentiate, between photons of multiple different wavelengths. According to some examples, the imaging device 118 may output, to the clinical device 120, at least one digital signal in response to the sensor(s) sensing the emission light. The digital signal(s) may represent at least one image of the sample 109, for instance. As used herein, the term “image,” and its equivalents, can refer to a two-dimensional (2D) group of pixels and/or a three-dimensional (3D) group of pixels (also referred to as “voxels” in 3D images). In various implementations, each pixel in an image has at least one value. In the case of an RGB image, each pixel has three values respectively corresponding to a red channel, a green channel, and a blue channel. Each pixel of the image(s) captured by the imaging device 118 depicts an area on the surface of the sample 109. In particular, pixels of the image(s) represent photons emitted from a detectable label linked to the synthetic nuclear tag 110. In some cases, the imaging device 118 includes and/or is communicatively coupled to a fluorescence microscope, such that the photons from the detectable label travel through an objective of the fluorescence microscope before being detected by at least one array of photosensors in the imaging device 118. The image(s), for example, include multiple images captured by the imaging device 118 at different focal lengths, angles, times, or any combination thereof. For instance, the clinical device 120 may generate a 3D image based on multiple 2D images sensed, by the imaging device 118, at parallel planes at particular intervals (e.g., z-stack imaging).

In addition, the clinical device 120 may identify one or more nuclei 104 of one or more cells 106 in the sample 109 by analyzing the image(s). For example, the clinical device 120 may identify a group of first pixels in the image(s) that corresponds to the nucleus 104 of the cell 106. Similarly, the clinical device 120 may identify a group of second pixels in the image(s) that corresponds to a nucleus of a second cell. In some examples, the clinical device 120 may use a segmentation technique to identify nuclei in the image(s). For instance, the clinical device 120 may use a machine learning (ML), thresholding, edge detection, region growing, or any other technique known in the art. In some examples, the clinical device 120 analyzes the intensity or color of pixels to identify regions of the image(s) that are part of a nucleus. In some examples, the clinical device 120 analyzes the intensity or color of neighboring pixels to identify edges that correspond to a boundary of a nucleus. In some examples, the clinical device 120 executes a ML model. The ML model may be developed (e.g., generated, trained, etc.) on the clinical device 120 or another computing device. The clinical device 120, for instance, may learn to identify patterns in training data by optimizing one or more metrics, values, or other types of parameters. The training data may include example images of example samples that may include one or more nuclei. After training, the ML model is executed on the clinical device 120, causing the clinical device 120 to use the optimized parameters in order to perform one or more tasks (e.g., identify nuclei in the image(s)). In various implementations, based on using the synthetic nuclear tag 110, training the ML model may require less computational resources than using conventional nuclear tags, for instance, due to the low off-target localization of the agent 102.

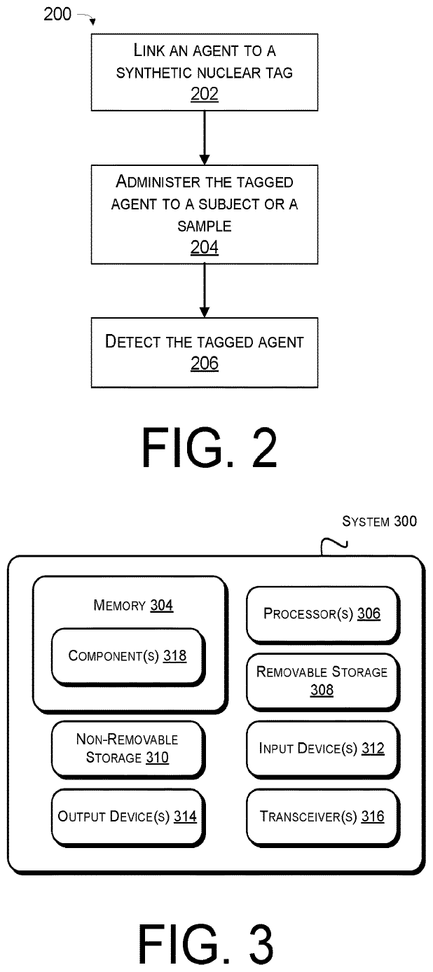

FIG. 2 illustrates an example process 200 for delivering an agent (e.g., the agent 102) to the nucleus (e.g., the nucleus 104) of a cell (e.g., the cell 106). In some examples, the agent is delivered to the nucleolus of the cell. The process 200 is performed by an entity, which may include one or more of a flow cytometer, a fluidic device (e.g., a microfluidic device), a computing device, or a user (e.g., a laboratory technician, care provider, or the like). According to some implementations, any of the steps of process 200 may be omitted.

At 202, the agent is conjugated to a synthetic nuclear tag (e.g., the synthetic nuclear tag 110) to produce a tagged agent (e.g., the tagged agent 116). The agent may include a peptide, a protein, a detectable label, a therapeutic agent, a vector, a nucleic acid, or the like. The agent may be linked to the synthetic nuclear tag. The agent may be linked to the synthetic nuclear tag by any technique disclosed herein. The agent, in some examples, is conjugated to a nucleic acid encoding the synthetic nuclear tag. In some examples, the entity conjugates the synthetic nuclear tag to the agent.

At 204, the tagged agent is administered to a subject (e.g., the subject 108) or a sample (e.g., the sample 109) that includes the cell. In some examples, the sample includes cells derived from the subject. In some examples, the sample includes modified cells, engineered cells, synthetic cells, chimeric cells, or the like. In some cases, the tagged agent is expressed in the subject or the sample. For instance, an expression construct that encodes the synthetic nuclear tag and the agent may be administered to the subject or the sample. Based on administering the expression construct, one or more cell(s) in the subject or sample may express the agent in the nucleus or the nucleolus of the cell(s).

The tagged agent may be administered to a subject suffering from a pathological condition to diagnose or treat the subject. For example, the tagged agent includes a therapeutic agent. The tagged agent may be administered to a subject suffering from a pathological condition to treat the subject. Treating subjects includes delivering therapeutically effective amounts. Therapeutically effective amounts include those that provide prophylactic treatments and/or therapeutic treatments.

An “effective amount” is the amount of a formulation used to result in a desired physiological change in the subject (e.g., the subject 108). For example, an effective amount can provide a change in a metric associated with the pathological condition (e.g., a severity or frequency of symptoms or a change in a marker associated with the pathological condition). Effective amounts are often administered for research purposes. Effective amounts disclosed herein can cause a statistically significant effect in an animal model or in vitro assay relevant to the assessment of a pathological condition's development or progression.

A “prophylactic treatment” includes a treatment administered to a subject (e.g., the subject 108) who does not display signs or symptoms of the pathological condition or displays only early signs or symptoms of the pathological condition such that treatment is administered for the purpose of diminishing or decreasing the risk of developing the pathological condition further. For example, a subject may be genetically predisposed to the pathological condition. The subject may be administered a gene therapy reagent linked to the synthetic nuclear tag (e.g., the synthetic nuclear tag 110) to modify one or more gene(s) associated with the pathological condition. Thus, a prophylactic treatment functions as a preventative treatment against the pathological condition. In particular implementations, prophylactic treatments reduce or delay physical symptoms associated with the pathological condition.

A “therapeutic treatment” includes a treatment administered to a subject (e.g., the subject 108) who displays symptoms or signs of the pathological condition and is administered to the subject for the purpose of diminishing or eliminating those signs or symptoms of the pathological condition. The therapeutic treatment can reduce, control, or eliminate the presence or activity of the pathological condition and/or reduce control or eliminate side effects of the pathological condition.

Function as an effective amount, prophylactic treatment or therapeutic treatment are not mutually exclusive, and in particular implementations, administered dosages may accomplish more than one treatment type.

In particular implementations, therapeutically effective amounts induce a reduced progression of the pathological condition. In particular implementations, the reduced progression includes reduced or stabilized symptoms, reduced or stabilized levels of one or more markers associated with the pathological condition, or a change in another metric known in the art.

For administration, therapeutically effective amounts (also referred to herein as doses) can be initially estimated based on results from in vitro assays and/or animal model studies. Such information can be used to more accurately determine useful doses in subjects of interest. The actual dose amount administered to a particular subject (e.g., the subject 108) can be determined by a physician, veterinarian, or researcher taking into account parameters such as physical and physiological factors including target, body weight, severity of condition, type of condition, stage of condition, previous or concurrent therapeutic interventions, idiopathy of the subject, and route of administration.

Useful doses, in some examples, are reported as a mass of the active ingredient (e.g., the agent 102 or a component of agent 102) per weight of the subject. Useful doses can range from 0.1 μg/kg to 2 mg/kg. In other examples, a dose can include 0.1 μg/kg, 0.2 μg/kg, 0.5 μg/kg, 1 μg/kg, 10 μg/kg, 20 μg/kg, 50 μg/kg, 100 μg/kg, 250 μg/kg, 500 μg/kg, 750 μg/kg, 1000 μg/kg, 0.1 to 5 mg/kg or from 0.5 to 1 mg/kg. In other examples, a dose can include 1 mg/kg, 1.25 mg/kg, 1.5 mg/kg, 1.75 mg/kg, 2 mg/kg, 5 mg/kg, or more.

In some examples, useful doses of nucleic acids (e.g., the agent 102) can be reported as infectious units (e.g., viral particles, viral genomes, plaque-forming units, transducing units, mass of the nucleic acid, or the like) per weight of the subject. Useful doses can range from 10² to 10²⁵ infectious units (IU)/kg. In other examples, a dose can include 10² IU/kg, 10³ IU/kg, 10⁴ IU/kg, 10⁵ IU/kg, 10⁶ IU/kg, 10⁷ IU/kg, 10⁸ IU/kg, 10⁹ IU/kg, 10¹⁰ IU/kg, 10¹¹ IU/kg, 10¹² IU/kg, 10¹³ IU/kg, 1014 IU/kg, 10¹⁵ IU/kg, 10¹⁶ IU/kg, 10¹⁷ IU/kg, 10¹⁸ IU/kg, 10¹⁹ IU/kg, 10²⁰ IU/kg, 10²¹ IU/kg, 10²² IU/kg, 10²³ IU/kg, 10²⁴ IU/kg, 10²⁵ IU/kg, or greater than 10²⁵ IU/kg.

Exemplary doses of cell-based compositions can include 10⁴ to 10⁹ cells/kg body weight, or 10³ to 10¹¹ cells/kg body weight. Therapeutically effective amounts to administer can include greater than 10² cells, greater than 10³ cells, greater than 10⁴ cells, greater than 10⁵ cells, greater than 10⁶ cells, greater than 10⁷ cells, greater than 10⁸ cells, greater than 10⁹ cells, greater than 10¹⁰ cells, or greater than 10¹¹ cells.

Therapeutically effective amounts can be achieved by administering single or multiple doses during the course of a treatment regimen (e.g., five times a day, four times a day, three times a day, twice a day, daily, every other day, every 3 days, every 4 days, every 5 days, every 6 days, weekly, every 2 weeks, every 3 weeks, monthly, every 2 months, every 3 months, every 4 months, every 5 months, every 6 months, every 7 months, every 8 months, every 9 months, every 10 months, every 11 months, or yearly). In particular implementations, the treatment protocol may be dictated by a clinical trial protocol or a US FDA-approved treatment protocol.

The compositions described herein can be administered by, for example, injection, inhalation, infusion, perfusion, lavage, or ingestion. Routes of administration can include intravenous, intradermal, intraarterial, intranodal, intravesicular, intrathecal, intraperitoneal, intraparenteral, intranasal, intralesional, intramuscular, oral, subcutaneous, and/or sublingual administration. Formulations are generally be administered by injection.

In various implementations of the present disclosure, a composition (e.g., the tagged agent 116, a nucleic acid encoding the synthetic nuclear tag 110, etc.) can be administered by, e.g., injection, infusion, perfusion, or lavage. Routes of administration can include bolus intravenous, intradermal, intraarterial, intraparenteral, intranodal, intralymphatic, intraperitoneal, intralesional, intraprostatic, intravaginal, intrarectal, topical, intrathecal, intratumoral, intramuscular, intravesical, and/or subcutaneous administration.

In some implementations, a composition is administered with a pharmaceutically acceptable carrier. Exemplary pharmaceutically acceptable carriers and formulations are disclosed in Remington's Pharmaceutical Sciences, 18^{th} Ed. Mack Printing Company, 1990. Moreover, formulations can be prepared to meet sterility, pyrogenicity, general safety and purity standards as required by United States FDA Office of Biological Standards and/or other relevant foreign regulatory agencies.

Exemplary generally used pharmaceutically acceptable carriers include at least one of a bulking agent or filler, a solvents or co-solvent, a dispersion media, a coating, a surfactant, an antioxidant (e.g., ascorbic acid, methionine, vitamin E, or the like), a preservative, an isotonic agent, an absorption delaying agent, a salt, a stabilizer, a buffering agent, a chelating agent (e.g., EDTA), a gel, a binder, a disintegration agent, a lubricants, or another carrier known in the art.

Exemplary buffering agents include citrate buffers, succinate buffers, tartrate buffers, fumarate buffers, gluconate buffers, oxalate buffers, lactate buffers, acetate buffers, phosphate buffers, histidine buffers, and trimethylamine salts.

Exemplary preservatives include phenol, benzyl alcohol, meta-cresol, methyl paraben, propyl paraben, octadecyldimethylbenzyl ammonium chloride, benzalkonium halides, hexamethonium chloride, alkyl parabens such as methyl or propyl paraben, catechol, resorcinol, cyclohexanol, and 3-pentanol.

Exemplary isotonic agents include polyhydric sugar alcohols including trihydric or higher sugar alcohols, such as glycerin, erythritol, arabitol, xylitol, sorbitol, and mannitol.

Exemplary stabilizers include organic sugars, polyhydric sugar alcohols, polyethylene glycol, sulfur-containing reducing agents, amino acids, low molecular weight polypeptides, proteins, immunoglobulins, hydrophilic polymers or polysaccharides.

At 206, in particular implementations, the tagged agent is detected. For instance, the tagged agent may include a detectable label. In various cases, the detectable label can be detected using a sensor. The tagged agent may be administered to the subject, and the sensor is configured to detect the detectable label in the subject or a sample derived from the subject. In some examples, the tagged agent is applied to a sample, and the sensor is configured to detect the detectable label in the sample.

FIG. 3 illustrates an example of a system 300 for performing various functions described herein. In some cases, the system 300 can represent the clinical device 120 described above with reference to FIG. 1.

As illustrated, the system 300 can include a memory 302. In various implementations, the memory 302 is volatile (including a component such as Random Access Memory (RAM)), non-volatile (including a component such as Read Only Memory (ROM), flash memory, etc.) or some combination of the two. The memory 302 may include various data, such as at least one component 304. The component(s) 304 can include methods, threads, processes, applications, or any other sort of executable instructions. For instance, the component(s) 304 may include instructions for performing any of the functionality described above with reference to FIGS. 1-2. The instructions, and various other elements stored in the memory 302, can also include other files and databases.

The memory 302 may include various instructions (e.g., among the component(s) 304), which can be executed by at least one processor 306 to perform operations. In some implementations, the processor(s) 806 includes a Central Processing Unit (CPU), a Graphics Processing Unit (GPU), or both CPU and GPU, or other processing unit or component known in the art.

The system 300 can also include additional data storage devices (removable and/or non-removable) such as, for example, magnetic disks, optical disks, or tape. Such additional storage can include removable storage 308 and non-removable storage 310. Tangible computer-readable media can include volatile and nonvolatile, removable and non-removable media implemented in any method or technology for storage of information, such as computer readable instructions, data structures, program modules, or other data. The memory 302, removable storage 308, and non-removable storage 310 are all examples of computer-readable storage media. Computer-readable storage media include, but are not limited to, RAM, ROM, EEPROM, flash memory or other memory technology, CD-ROM, Digital Versatile Discs (DVDs), Content-Addressable Memory (CAM), or other optical storage, magnetic cassettes, magnetic tape, magnetic disk storage or other magnetic storage devices, or any other medium which can be used to store the desired information and which can be accessed by the system 300. Any such tangible computer-readable media can be part of the system 300.

The system 300 also can include input device(s) 312, such as a keypad, a cursor control, a touch-sensitive display, voice input device, etc., and output device(s) 314 such as a display, speakers, printers, etc. These devices are well known in the art and need not be discussed at length here. In particular implementations, a user can provide input to the system 300 via a user interface associated with the input device(s) 312 and/or the output device(s) 314.

The system 300 can also include one or more wired or wireless transceiver(s) 316. For example, the transceiver(s) 316 can include a Network Interface Card (NIC), a network adapter, a Local Area Network (LAN) adapter, or a physical, virtual, or logical address to connect to the various base stations or networks contemplated herein, for example, or the various user devices and servers. To increase throughput when exchanging wireless data, the transceiver(s) 316 can utilize Multiple-Input/Multiple-Output (MIMO) technology. The transceiver(s) 316 can include any sort of wireless transceivers capable of engaging in wireless, Radio Frequency (RF) communication. The transceiver(s) 316 can also include other wireless modems, such as a modem for engaging in Wi-Fi, WiMAX, Bluetooth, or infrared communication.

In some implementations, the transceiver(s) 316 can be used to communicate between various functions, components, modules, or the like, that are included in the system 300. For instance, the transceiver(s) 316 can be used to transmit data between the system 300 and the imaging device 118, an external user equipment (UE), analysis device, or the like.

### EXPERIMENTAL EXAMPLE 1

This Example describes a new NLS genetic tag strategy named “Arginine-rich NLS” or “ArgiNLS”, that improves nuclear localization of genetically encoded FPs and enhances image segmentation of single-cells through a poly-arginine driven nucleolar enrichment mechanism. This method does not alter neural physiology or mouse behavior after standard AAV expression windows and is therefore compatible with AAV-paired behavioral procedures. It is functionally modular when using spectrally separate FPs and possesses equal efficacy when expressed within genetically distinct cortical cell-types. Moreover, it significantly improves two- and three-dimensional image segmentation that decreases computational expense associated with ML-based segmentation.

### Materials and Methods

Mice: Adult male and female mice (8-12 weeks old) were used for all experiments unless otherwise noted. Animals were group housed on a reverse 12-hr light-dark cycle (0700 OFF/1900 ON) and had access to water and food ad libitum. All experimental procedures were performed in accordance with University of Washington IACUC guidelines. Wild-type and transgenic mice lines were purchased from the following vendors: C57B16/J (JAX, 000664); CD-1 (Charles River, Crl:CD1(ICR)); vGlut1-Cre (Slc17a7-IRES2-Cre-D; JAX, 023527); vGat-Cre (Vgat-ires-cre knock-in (C57BL/6J); JAX, 028862); vGat-Flp (Slc32a1-IRES2-FlpO-D knock-in; JAX, 031331).

Fluorescent protein-expressing AAV Vector construction: Untagged and NLS-tagged fluorescent proteins were cloned through PCR amplification with primers with or without NLS extension overhangs (Integrated DNA Technologies). The monopartite SV40nls coding tag sequence used was from amino acids 126-132 of large T antigen (Betapolyomavirus macacae; NCBI Reference sequence: NP_043127.1). Amino acids 156-172 of the bipartite mouse nucleoplasmin2 NLS (NCBI accession number: NP_001398932.1) was used for NPM2nls. Each sequence and the addition of an in-frame 7 polyarginine stretch was codon-optimized for expression in mouse cells. The following plasmid DNA plasmids were purchased from Addgene and used for PCR templates: pAAV-CAG-NLS-GFP (104061) for all NLS-EGFP tag variants; pAAV-EF1α-oScarlet (137135) for oScarlet and ArgiNLS-oScarlet; pAAV-RAM-d2TTA::TRE-NLS-mKate2-WPREpA (84474) for mKate2 and ArgiNLS-mKate2; pmiRFP670-N1 (79987) for miRFP670 and ArgiNLS-miRFP670; and pCS-H2B-EGFP (53744) for H2B-EGFP. For constitutive expression AAV construction, all fluorescent proteins and variants were subcloned into pAAV-Ef1α-DIO-Synaptophysin-GcaMP6s (105715) at XhoI/EcoRV restriction enzyme sites. For Cre-dependent expression AAV construction, all fluorescent proteins and ArgiNLS-tagged variants were subcloned into pAAV-CAG-FLEX-GFP (59331) at KpnI/XhoI restriction enzyme sites. For Flp-dependent expression AAV construction, ArgiNLS-mKate2 was subcloned into pAAV-CAG-fDIO-mNeonGreen (99133) at KpnI/XhoI restriction enzyme sites. All cloned insert sequences of final plasmids used in the manuscript were sequence verified by Sanger sequencing (Genewiz, Seattle, WA).

TABLE 1
Available ArgiNLS-tagged genetic constructs for optimized cell counting.

| Construct | Promoter | FP Gene | Ex/Em | Cond.* |
|---|---|---|---|---|
| AAV-EF1α-ArgiNLS-EGFP | EF1α | EGFP | 488/507 | 1 |
| AAV-EF1α-ArgiNLS-AausFP1 | EF1α | AausFP1 | 504/510 | 1 |
| AAV-EF1α-ArgiNLS-mGreenLantern | EF1α | mGreenLantern | 503/514 | 1 |
| AAV-EF1α-ArgiNLS-mVenus-Q69M (ME) | EF1α | mVenus ME | 515/528 | 1 |
| AAV-EF1α-ArgiNLS-oScarlet | EF1α | oScarlet | 569/594 | 1 |
| AAV-EF1α-ArgiNLS-mKate2 | EF1α | mKate2 | 588/633 | 1 |
| AAV-EF1α-ArgiNLS-miRFP670 | EF1α | miRFP670 | 642/670 | 1 |
| AAV-CAG-FLEX-ArgiNLS-EGFP | CAG | EGFP | 488/507 | 2 |
| AAV-CAG-FLEX-ArgiNLS-AausFP1 | CAG | AausFP1 | 504/510 | 2 |
| AAV-CAG-FLEX-ArgiNLS-mGreenLantern | CAG | mGreenLantern | 503/514 | 2 |
| AAV-CAG-FLEX-ArgiNLS-mVenus-Q69M (ME) | CAG | mVenus ME | 515/528 | 2 |
| AAV-CAG-FLEX-ArgiNLS-oScarlet | CAG | oScarlet | 569/594 | 2 |
| AAV-CAG-FLEX-ArgiNLS-mKate2 | CAG | mKate2 | 588/633 | 2 |
| AAV-CAG-FLEX-ArgiNLS-miRFP670 | CAG | miRFP670 | 642/670 | 2 |
| AAV-CAG-fDIO-ArgiNLS-mKate2 | CAG | mKate2 | 588/633 | 3 |

Table 1 lists the available ArgiNLS-tagged genetic constructs for optimized cell counting. *Condition 1: Constitutive; Condition 2: Ore-dependent; Condition 3: Flp-dependent

Characterization of NLS tag nuclear localization in vitro: Immortalized embryonic mouse hippocampal cells (Cedarlane; mHippoE-1; cat. CLU198)) or mouse Neuroblastoma 2A cells (ATCC; CCL-131) were cultured in DMEM (high glucose and L-glutamine; Gibco 11965-092), supplemented with 10% fetal bovine serum (Gibco; 1600) and 1% peniciIlin-streptomycin (Invitrogen; 15140122) at 3700 in 5% CO₂. Cells were sub-cultured following manufacturer's instructions. For transient DNA transfections, cells were seeded onto Poly-L-lysine-coated glass coverslips (Electron Microscopy Science, 72292-08) in a 24-well plate format. Once cells reached 80% confluence, transient transfections were performed in duplicate with 250 ng of plasmid DNA per construct using Lipofectamine 3000 transfection reagent (ThermoFisher Scientific, L3000015). After 48 h, cells were fixed with formalin, counterstained with DAPI, and mounted onto glass slides. All NLS tagged variants were co-transfected with a spectrally separate untagged fluorescent protein to fluorescently label total cellular area in the following combinations: all NLS tagged EGFP constructs with AAV-EF1α-mKate2; all NLS tagged RFP constructs with p-EGFP-N1 (Addgene; 6085-1). To qualitatively verify Cre-dependent ArgiNLS variant expression, each plasmid was co-transfected with pCAG-Cre (Addgene; 13775), respectively. To qualitatively verify Flp-dependent ArgiNLS-mKate2 expression, FlpO was PCR-amplified and subcloned from PGK-NLS-FlpO-bpA (a gift from Dr. Phillipe Soriano; Raymond & Soriano, PLOS ONE 2:e162, 2007) into ptCAV-12a (Kremer et al., J Virol74:505-512, 2000) at Agel/EcoRV restriction sites to create the FpO-expressing ptCAV-Flp0-12a which was used for co-transfections. Images of all transfection conditions were acquired with a Keyence BZ-X800E inverted fluorescence microscope using a 20×/0.75 NA objective (Nikon). The effects of experimental NLS and ArgiNLS tags on cellular localization of fluorescence was quantified amongst single cells (n=5 randomly selected cells/3 replicate cultures) in a blinded fashion using ImageJ. Accordingly, manual segmentations of the nucleus as defined by DAPI fluorescence, and the whole cell as defined by untagged co-transfected plasmid fluorescence, were created in synchronized duplicate images of the target channel. Corrected total nuclear or whole cell fluorescence (CTNF or CTCF, respectively) were separately calculated using area, mean, and integrated density measurements within the following equation: CTF=Integrated density−(Area*Mean background). Percent nuclear localized fluorescence was then calculated for each cell as CTNF/CTCF*100.

AAV Production: Php.eB and retroAAV2 packaging plasmid construction: A single packaging plasmid for the production of PHP.eB viruses, pDG_PHP.eB, was constructed by subcloning the PHP.eB capsid from pUCmini-iCAP-PHP.eB (Addgene, 103005) into pDG1 (Grimm et al., Hum Gene Ther9:2745-2760, 1998) plasmid to replace the AAV1 capsid at Clal/Swal restriction enzyme sites. The final vector was produced at 1 mg/mL by Nature Technologies (Lincoln, Nebraska). A single packaging plasmid for retroAAV2 was created by subcloning the retroAAV2 capsid (Tervo et al., Neuron 92:372-382, 2016) into the pDG plasmid in a manner identical to pDG_PHP.eB to create pDG_retroAAV2. The pDG_PHP.eB plasmid is publicly available through Addgene.

AAV generation: HEK293T cells were transfected with 25 μg AAV vector plasmid and 50 μg packaging vector (pDG_PhP.eB or pDG2_retroAAV2) per 15 cm plate. Two days after transfection, cells were harvested and subjected to three freeze-thaw cycles. The supernatant was transferred to a Beckman tube containing a 40% sucrose cushion and spun at 27,000 rpm overnight at 4 °C. Pellets were resuspended in CsCl at a density of 1.37 g/ml and spun at 65000 rpm 4 hours at 4 °C. 1 ml CsCl fractions were run on an agarose gel, and genome-containing fractions were selected and spun at 50000 rpm overnight at 4 °C. The 1 ml fractions were collected again, and genome containing fractions were dialyzed overnight. The filtered solution was transferred to a Beckman tube containing a 40% sucrose cushion and spun at 27,000 rpm overnight at 4 °C. The pellet (containing purified AAV) was resuspended in 100 μl 1×HBSS. Virus was aliquoted and stored at −80 °C until use

AAV genome copy titration: Two methods were employed to titrate viral DNA genome copies of AAV preparations: dPCR by Stanford's Gene Vector and Virus Core for initial preparations, and in-house SYBR green-based qRT-PCR for subsequent preparations. For dPCR, the Qiacuity 5-plex system was employed which uses random distribution of template in available partitions to detect and measure fluorescence emission from target amplicons. The virus is was first made DNA ready by adding 8 μl of Biosearch Quickextract solution to 2 μl of sample and thermocycling at 65 °C for 6 min and 98 °C for 2 min. Extracted DNA was then combined at 100×, 10,000×, and 5,000,000× dilutions with Qiacuity ProbePCR master mix and XFP primers (forward: AGCAAAGACCCCAACGAGAA (SEQ ID NO: 7); reverse: GGCGGCGGTCACGAACT (SEQ ID NO: 8)), and reaction mixes were subjected to a dPCR thermocycling program: 95 °C for 2 min, and 50 cycles of 15 sec at 95 °C and 30 sec of 60 °C. Earlier determined titer of AAV-45 was included as internal control along with a No Template Control. The concentration ‘copies/μl’ is then used to calculate the genome copies per mL. For qRT-PCR, 5 μl of Dnase I-treated AAV at 25000× and 125000 dilutions or linearized standard plasmid DNA (2×10⁴-2×10⁸ molecules) were combined into master mixes containing SsoAdvanced Universal SYBR Green (Bio-Rad, 1725271) and primer sets targeting either the WPRE3 sequence (forward: CTGGTTAGTTCTTGCCACGG (SEQ ID NO: 9); reverse: GTTGGGCACTGACAATTCCG (SEQ ID NO: 10)) for titration of constitutive viruses, or the WPRE sequence (forward: AAGGAAGGTCCGCTGGATTG (SEQ ID NO: 11); reverse: AAAGTCCCGGAAAGGAGCTG (SEQ ID NO: 12)) for titration of conditional viruses. 20 μl reaction mixes were then subjected to the following thermal cycling protocol using a Bio-Rad CFX Connect Real-Time PCR System: 2 min at 98 °C, and 38 cycles of 15 sec at 98 °C and 20 sec at 60 °C. Viral genome copies/μl were calculated in Microsoft Excel based off of plasmid DNA standard curves that passed quality control of an R2 of 0.99 and PCR efficiencies of 100%.

Stereotaxic viral injections: All stereotaxic surgeries were performed under aseptic conditions. Briefly, animals were anesthetized with isoflurane prior to and during placement and surgery in a stereotaxic instrument (David Kopf Instruments). Scalp incisions were made to expose the skull surface and virus was injected intracranially using a 32 gauge 0.5 μl Neuros syringe (Hamilton Co.) at the following coordinates from bregma of the mouse brain: SSpBF: −1.67 anteroposterior, 2.75 mediolateral, −0.9 dorsoventral; Nac (10-degree angle): 1.5 anteroposterior, 1.7 mediolateral, −4.5 dorsoventral. For NLS nuclear fluorescence comparisons in defined cell-types, 400 nl of AAV.eB-EF1α-ArgiNLS-EGFP (7×10¹² GC/ml) or AAV.eB-EF1α-Sv40nls-EGFP (7×10¹² GC/ml) was co-injected with 100 nl of AAV.eB-CAG-FLEX-tdTomato (Addgene, 28306; 7×10¹³ GC/ml) into the SSpBF of homozygous vGlut1-Cre or vGat-Cre mice at a 0.1 μl/min delivery rate. For qualitative characterization of conditional ArgiNLS-tagged fluorescent proteins, 500 nl of AAV.eB-CAG-FLEX-ArgiNLS-EGFP (1×10¹² GC/ml) and AAV.eB-CAG-fDIO-ArgiNLS-mKate2 (1×10¹² GC/ml) virus was injected into the SSpBF of separate heterozygous vGat-Cre or vGat-Flp mice at a 0.1 μl/min delivery rate. For characterization of retrograde ArgiNLS-tagged fluorescent protein expression, 200 nl of retroAAV2-EF1α-ArgiNLS-mKate2 (1.5×10¹³ GC/ml) virus was injected into the Nac of C57BI6/J mice at a 0.02 μl/min delivery rate. All surgerized animals received 1 mg/ml ketoprofen analgesic and 500 μl saline subcutaneously followed by recovery in their home cage placed above a warm heating pad. All animals receiving stereotaxic injections were sacrificed for histological analysis at 2 weeks post-injection.

Characterization of ArgiNLS—EGFP nuclear localization in-vivo: Local AAV-injected mice were terminally anesthetized with isoflurane and transcardially perfused with phosphate-buffered saline (PBS) followed by 10% neutral buffered formalin (Sigma, HT501128). Brains were extracted, post-fixed overnight in 4 °C, and cryoprotected in 30% sucrose at 4C. Cryoprotected brains and livers were histologically sectioned on a freezing cryostat (Leica Biosystems of Wetzlar, Germany), where brains were sliced in coronal orientation at 50 μm in 6 equal series and livers were sectioned at 10 μm thickness. Sections were counterstained with DAPI (Millipore Sigma; 10236276001) and then mounted with VectaShield mounting media (Vector Laboratories; H-1400-10). Mounted sections were imaged using a Ziess LSM880 confocal microscope. Confocal images were captured with either 10×/0.45 NA (Zeiss) or 40×/1.2 NA (Zeiss) objectives. All scans were acquired in 16-bit with 2.05 μs pixel dwell time and line 8 averaging in a 1024×1024 dimension. 3-channel DAPI/EGFP/tdTomato images were captured using the following excitation/emission filters: DAPI: 410-585 nm; EGFP: 493-564 nm; tdTomato: 566-691 nm. Consistent laser power and gain settings were used across all conditions. 40× scans used for quantification were acquired in Z-stacks consisting of 3 μm optical steps across 15 um total tissue depth. Maximal z-projections were created in ImageJ and used for quantification. The effects of SV40nls and ArgiNLS tags on cellular localization of EGFP fluorescence was quantified in EGFP+/tdTomato+single cells (n=5 randomly selected cells/3vGlut1-Cre orvGat-cre animals) from 40× images in a blinded fashion using ImageJ in a manner similarly described for in-vitro quantification. Briefly, manual segmentations of the nucleus as defined by DAPI fluorescence, and the soma as defined by Cre-dependent tdTomato fluorescence, were created in synchronized duplicate images of the target EGFP channel. Since different levels of extracellular background fluorescence were observed at the injection site amongst SV40nls- and ArgiNLS-EGFP injections, DAPI-guided extracellular background fluorescence images and measurements from the un-injected contralateral side of the SSpBF were used for background subtraction. These values were also used to compare against the extracellular fluorescence background measurements taken within each injection site of all animals to perform ipsilateral versus contralateral extracellular background fluorescence comparisons. Axonal fluorescence was quantified from additional 20× image stack z maximal z-projections acquired in the corpus callosum from vGlut1-Cre samples. For quantification, an adaptive manual threshold was applied to isolate measurements to axons only using the Huang method in ImageJ on 608×1024 FOVs. Corrected total axonal fluorescence (CTAF) was then calculated for each channel as Integrated density−(Area*mean background). EGFP+CTAF was internally normalized for each sample to vGlut1-Cre:tdTomato+CTAF and expressed as a percent.

Retroorbital viral injections: 1×10¹¹ GC of AAV.eB virus was diluted up to 100 μl with sterile saline and injected intravenously into the retro-orbital sinus of mice receiving brief isoflurane anesthesia. All RO-injected animals were sacrificed at 3 weeks post-injection for histological analysis.

Performance benchmarking of systemically labeled single-cell segmentation with ArgiNLS-tagged fluorescence: Mice RO-injected with AAV.eB-EF1α-ArgiNLS-EGFP or AAV.eB-EF1α-Sv40nls-EGFP were terminally anesthetized with isoflurane and transcardially perfused and histologically processed as described above. Single-plane epifluorescent images of entire coronal planes at AP 2.11 and −0.52 were acquired in montages with a Keyence BZ-X800E inverted fluorescence microscope using a 10×/0.45 NA objective (Nikon). Consistent DAPI and EGFP channel exposure settings were applied across conditions. Montages were stitched using BZ-X800 Analyzer software (Keyence, v 1.1.1.8) and composite images were created in ImageJ and exported as TIFF images. Machine learning classifiers for ArgiNLS- and SV40nls-EGFP labels were created using random forest classification with QuPath (P. Bankhead, et al., Sci Rep 7, 16878 (2017)) software applied to composite TIF images.

QuPath Classifier Training: Machine learning classifiers for ArgiNLS and SV40nls-tagged EGFP image segmentation of single cells were trained using QuPath (Bankhead et al., Sci Rep 7:16878, 2017) applied to composite TIF images. 25 px×25 px (625 px²) regions of interest (ROIs) labeled as ‘DAPI’ or ‘GFP’ were used as training input data. The object classification function was used to create classifiers with the following train object classifier parameters: object filter=all detections; classifier=random trees (Rtrees), features=all measurements, classes=all classes, and training=unlocked annotations. Cell detection parameters consisted of: Setup parameters detection channel=DAPI; nucleus parameters background radius=0 px; median filter radius=0 px; sigma=3 px; minimum area=10 px2; maximum area=200 px2; intensity threshold=50 and split by shape; cell expansion=0 px; general parameters=smooth boundaries and make measurements. The live update function was used to observe classifier training, and each training iteration included the addition of 2 training ROIs. The first training iteration for each classifier included both a ‘DAPI’ and ‘GFP’-labeled training ROI. Total iterations to reach maximal performance for the ArgiNLS classifier was 10 and included a combination of 7 DAPI and 2 GFP training ROIs. Total iterations to reach maximal performance for the SV40nls classifier was 28 and included a combination of 22 DAPI and 6 GFP training ROIs.

Classifier Performance Quantification: Additional 100 px×100 px (10,000 px²) FOVs were created (4 per AP) to quantify classifier performance at every training iteration. FOV placement varied ranging from regions with small to large density of labeled cells. Labeled cells in each FOV were manually scored with a single point separately by 3 biological expert raters, and classifier training was repeated, as described above. Both manual and filled automated segmentations fluorescent label were overlaid for each FOV in QuPath and GT(+)/classifier(−), GT(−)/classifier(+), and GT(+)/classifier(+) co-segmentations were hand scored for each FOV at every training iteration. Data was recorded in Microsoft Excel and used to calculate precision, recall, and F1 scores.

Brain-wide quantification and characterization of ArgiNLS-EGFP labeled single cells: Two independent approaches were applied to systematically quantify and characterize single-cell labeling across the intact mouse brain with ArgiNLS-tagged EGFP: 1) iDISCO+solvent-based brain clearing and EGFP staining based on methods previously described (Renier et al., Cell 165(7):1789-1802, 2016; Madangopal et al., PNAS 119(45): e2209382119, 2022 doi.org/10.1073/pnas.2209382119) and 2) SHIELD aqueous-based brain clearing (Park et al., Nature Biotech. 37:73-83, 2019).

iDISCO+whole-brain processing: Mice RO-injected with AAV.eB-EF1α-ArgiNLS-EGFP (n=4) or AAV.eB-EF1α-Sv40nls-EGFP (n=4) were sacrificed and brains were post-fixed as described above. iDISCO+was performed, beginning with a pretreatment of 1×PBS washes (3×30 min; RT) followed by dehydration using ascending incubations of methanol (MeOH; Sigma, 34860-4L-R) concentrations (1 h each step; RT rotating): 20%, 40%, 60%, 80%, 100%, and 100%. Samples were next delipidated with a 66% dichloromethane (DCM; Sigma, 270997-12X100ML)/34% MeOH solution (ON, RT rotating), and washed the following day in 100% MeOH (2×1 h, RT rotating). Brains were then bleached in a chilled solution consisting of 5% hydrogen peroxide (30% Hydrogen peroxide solution; Sigma, 21673-500ML)/methanol (ON, 4 °C). Bleached brains were then rehydrated using descending incubations of MeOH concentrations (1 h each step; RT): 80%, 60%, 40%, 20%, and finally washed in 1×PBS buffer containing 0.5% TritonX-100 (Sigma, T9284-1L) (2×1 h; RT rotating). To immunostain brains for EGFP, pre-treated samples were first permeabilized in a 1×PBS buffer containing 0.5% TritonX-100/1.4% glycine (Sigma, G7126-1KG)/20% dimethyl sulfoxide (DMSO; Sigma, D128-1) (2 days, 37 °C rotating) followed by blocking of non-specific binding through incubation with a 1×PBS buffer containing 0.5% TritonX-100/6% normal donkey serum (NDS; Jackson ImmunoResearch, 017-000-121)/10% DMSO (2 d, 37 °C rotating). EGFP primary antibody (chicken polyclonal; Aves, GFP1010) labeling was next performed by applying a 1:200 dilution in a 1×PBS buffer containing 0.5% Tween-20/10 ug/mL heparin (Sigma, H3393-250KU)/3% NDS/5% DMSO (7 days, 37 °C rotating). Excess antibody was then washed with 1×PBS buffer containing 0.5% Tween-20/10 μg/mL heparin (4-5 times×2 days, 37 °C rotating). Secondary antibody (donkey anti-chicken Alexa Fluor-647-conjugated AffiniPure F(ab′)2; Jackson ImmunoResearch; 703-606-155; Lot: 157044) staining was then performed by applying a 1:100 dilution in a 1×PBS buffer containing 0.5% Tween-20/10 ug/mL Heparin/3% NDS/5% DMSO (7 days, 37 C rotating). Excess secondary antibody was washed with 1×PBS buffer containing 0.5% Tween-20/10 μg/mL heparin (4-5 times×3 days, 37 °C rotating). Tissue clearing then began with dehydration using ascending incubations of MeOH concentrations (1 h each step; RT rotating): 20%, 40%, 60%, 80%, 100%, and 100% followed by delipidation with a 66% DCM/34% MeOH solution (3 h, RT rotating). Next, MeOH was washed with 100% DCM (2×15 min; RT rotating). Finally, samples were index matched for at least 1 day in DiBenzyl Ether (DBE; Sigma, 108014-1KG) prior to imaging.

iDISCO+-processed volumetric imaging: EGFP-stained and cleared whole-brains were imaged in DBE using an UltraMicroscope II light-sheet fluorescent microscope (LSFM) with Infinity Corrected Objective Lenses (Miltenyi Biotec). The microscope configuration consisted of a 1.1×/0.1 NA MI PLAN objective (LaVision BioTec), non-corrected dipping cap, and an Andor Zyla sCMOS camera. All samples were secured on a 3D-printed sample platform and positioned in horizontal orientation with the cortical surface facing up. 2-channel image acquisition was performed in 1 FOV tile for autofluorescence (Excitation: 488 nm laser, Emission: 535/43 bandpass filter) and EGFP stain (Excitation: 647 nm laser, Emission: 690/50 bandpass filter) in separate scans (scan order: z-x-y) at a near-isotropic pixel resolution of 5.91 μm X×5.91 μm Y×6 μm Z. The following fixed acquisition parameters were applied across all samples using ImspectorPro software (Miltenyi Biotec; v 7.1.16): laser power=20 (488 channel), 30 (647 channel); exposure=100 ms (488 channel), 136 ms (647 channel); sheet NA=0.16; sheet thickness=3.89 μm; sheet width=100%; zoom=1×; dynamic horizontal focus=5 (647 channel only); dynamic horizontal focus processing=contrast adaptive; merge light-sheet=blend.

SHIELD whole-brain processing: The SmartClear full active pipeline protocol (LifeCanvas Technologies, v5.05) was followed for aqueous-based brain clearing and mounting. Accordingly, RO- or locally-injected mice were sacrificed and brains were post-fixed as described above. Brain samples were first incubated in freshly prepared SHIELD OFF solution, consisting of SHIELD buffer and epoxy solution (4 days, 4 °C shaking), followed by SHIELD ON buffer (1 day, 37 °C shaking). Samples were next equilibrated in Delipidation buffer (ON, RT shaking). Prior to active clearing, the SmartClear II Pro electrophoresis system (LifeCanvas Technologies) containing Delipidation buffer was equilibrated to 42 °C and new membranes were installed. Samples were then transferred into mesh bags and placed into clearing chambers. Active clearing was performed on 4-sample batches using the Gentle Mode program (1500 mA/42 °C; 1 day). After clearing, samples were removed from mesh bags and index matched first in 50% EasyIndex (1 day; 37 °C slow shaking) followed by 100% EasyIndex (1 day; 37 °C slow shaking). Index matched samples were embedded on custom 3D-printed sample mounts in EasyIndex containing 2% w/v agarose (Sigma, A5030), and incubated post-embedding in 100% EasyIndex (ON, 37 °C). Embedded samples were stored in 100% EasyIndex (RT) in the dark until imaging.

SHIELD-processed volumetric imaging of native fluorescence: Native signal from aqueous-based cleared brains were imaged horizontally using the SmartSPIM LSFM (LifeCanvas Technologies) (A. Li, et al., Science 330, 1404-1408 (2010)) at 4 um isotropic pixel resolution. All samples were imaged in 488 and 563 channels to acquire native fluorescence from AAV expression from green- or red-emitting FPs, and background fluorescence in the channel without AAV expression for 3D rendering purposes. Laser power and acquisition settings were optimized individually towards each proof-of-concept sample, whereas settings for titrated AAV.eB-EF1α-ArgiNLS-oScarlet samples were held constant across conditions.

Whole-brain image processing: Brain-wide ArgiNLS—versus SV40nls-tagged EGFP cell count comparisons: Volumetric LSFM-acquired images were processed for whole-brain template registration, automated cell segmentation, and regional segmentation as previously described with modifications (R. Madangopal, et al., Biorxiv. [Preprint](2022)). First, the Unified brain template and atlas (Chon et al., Nature Comm. 10: Art. No. 5067, 2019) were employed for registration and regional segmentation, respectively. Second, 3D ML classification of single-cells from SV40nls-EGFP or ArgiNLS-EGFP stains was applied through the creation of separately trained classifiers using random forest classification algorithm through ilastik software(Y. Kim, et al., Cell Rep 10, 292-305 (2015)). Classifier Performance was quantified in FOVs in regions with small to large density of labeled cells as similarly described previously on non-ML segmented datasets (S. Corsetti, et al., J Neurosci Methods 319, 16-27 (2019)). Labeled cells in each FOV were manually segmented separately by 3 expert raters. Data from ImageJ processing was recorded in Microsoft Excel and used to calculate precision, recall, and F1 scores. [0123]3D Visualization and ML: Zeiss arivis Pro (formerly, Vision4D) software was used to generate 3D renderings of brain samples throughout this Example.

Slice electrophysiology: vGlut1-Cre mice injected with ArgiNLS-EGFP and Cre-dependent tdTomato virus were allowed 3-4 weeks recovery after surgery to allow for viral expression. All solutions were continuously bubbled with O₂/CO₂. Coronal (200 μm) brain slices were prepared from 11-13 week old mice in a slush NMDG cutting solution (in mM: 92 NMDG, 2.5 KCl, 1.25 NaH₂PO₄, 30 NaHCO₃, 20 HEPES, 25 glucose, 2 thiourea, 5 Na-ascorbate, 3 Na-pyruvate, 0.5 CaCl₂, 10 MgSO₄, pH 7.3-7.4 (Ting et al., Methods Mol Biol 1183:221-242, 2014). Slices recovered for 12 min in the same solution in 32 °C water bath. Slices were then transferred to a room temperature HEPES-aCSF solution (in mM: 92 NaCl, 2.5 KCl, 1.25 NaH₂PO₄, 30 NaHCO₃, 20 HEPES, 25 glucose, 2 thiourea, 5 Na-ascorbate, 3 Na-pyruvate, 2 CaCl₂, 2 MgSO₄). Slices recovered for an additional 60 min.

Whole-cell patch-clamp recordings were made using an Axopatch 700B amplifier (Molecular Devices) with sampling at 10 KHz and filtering at 1 kHz. EGFP+ and/or tdTomato+cells were visually identified via fluorescence for patching with electrodes at 4-6 MO. Series resistance was monitored during all recordings with changes in resistance +10% qualifying the cell for exclusion. Recordings were made in aCSF (in mM: 126 NaCl, 2.5 KCl, 1.2 NaH₂PO₄, 1.2 MgCl₂ 11 D-glucose, 18 NaHCO₃, 2.4 CaCl₂) at 32 °C continually perfused over slices at a rate of 2 ml/min. Barrel cortex glutamate neurons were identified by fluorescence. Electrodes were filled with an internal solution containing (in mM): 130 potassium gluconate, 10 HEPES, 5 NaCl, 1 EGTA), 5 Mg²⁺/ATP, 0.5 Na+-GTP, pH 7.3, 280 mOsmol. Excitability I/V curves (−20-180 pA, 10 pA steps, 500 ms) were measured in I-Clamp mode after gaining whole cell access. All data were analyzed offline using Clampfit (Molecular Devices). Excitability was measured as the total number of events during each current step.

Behavioral health battery: To screen for behavioral effects from systemic AAV.eB expression from experimental viruses the following battery of behavioral tests were performed in chronological order on the same mice.

Open field testing: Mice were placed in a large circular arena (120 cm diameter) in dim light and activity was recorded for a period of 10 minutes. Data were analyzed using Ethovision software, where time in center, time on edge, and total distance were calculated.

Elevated Plus maze (EPM): The EPM is plus-shaped arena in which two maze arms are sheltered by enclosed walls, and two arms are open platforms. Each arm length is 15 cm, with a center area of 49 cm². Activity was recorded for a period of 10 min and analyzed using Ethovision software.

Locomotor activity: Baseline locomotion was measured using locomotion chambers (Columbus instruments) that use infrared beam breaks to calculate ambulatory activity. Mice were singly housed in Allentown cages with reduced corncob bedding and provided with ad libitum access to food and water. Locomotion was monitored continuously over the course of 3 nights and 2 days.

Statistics: Raw data provided throughout the manuscript represent mean±S.E.M. values. With the exception of voxel statistical tests (performed in ClearMAP software (G. Cai, et al., Neurobiol Stress 20, 100478 (2022))), all other statistical analyses and data graphing was performed with Prism 10 software (GraphPad). Alpha levels for statistical significance were set at 0.05 for p and q-values. Probability levels of statistical significance from group mean comparisons are indicated in figures with an asterisk labeling convention of: *p<0.05; **p<0.01; **p<0.01; ****p<0.001. Two group comparisons were analyzed using two-tailed, unpaired Student's t-test. One- or two-way ANOVA analysis of main effects were used to compare group means with one of two independent variables, respectively. Posthoc tests were chosen on an experiment-by-experiment basis and indicated in each figure legend. Region-by-region analysis of whole-brain datasets were performed using FDR-corrected multiple t-tests by the two-stage step-up method of Benjamini, Krieger, and Yekutieli (A. V. Roland, et al., Biol Psychiatry (2023)) on uncollapsed levels of all subregions from major brain ontologies.

### Results

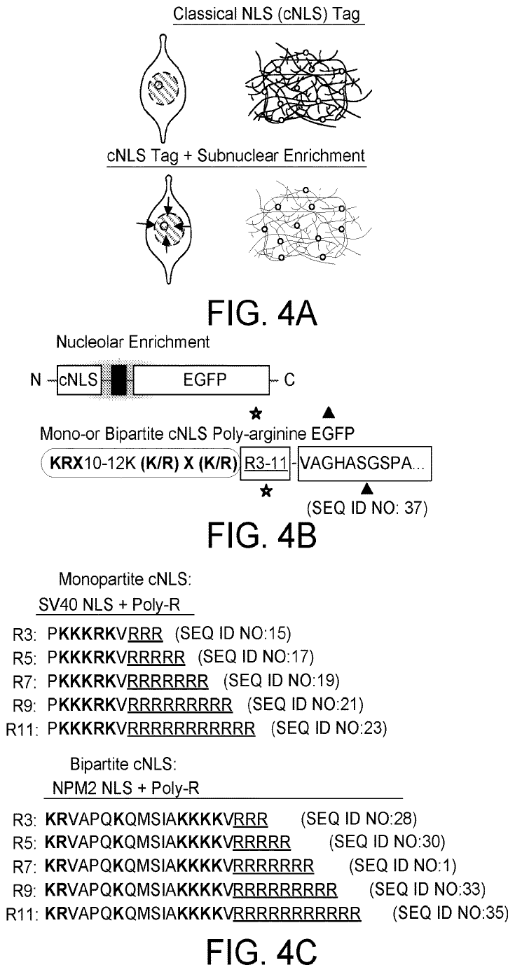

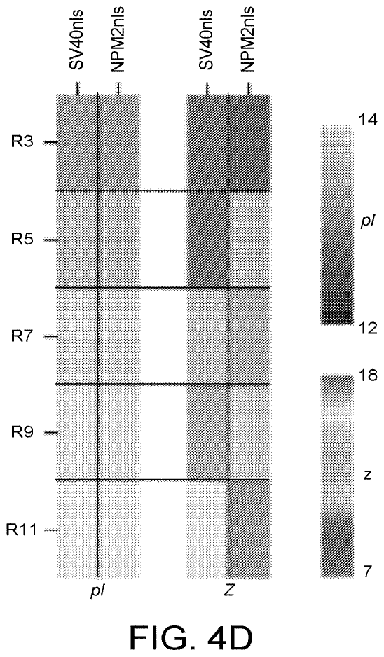

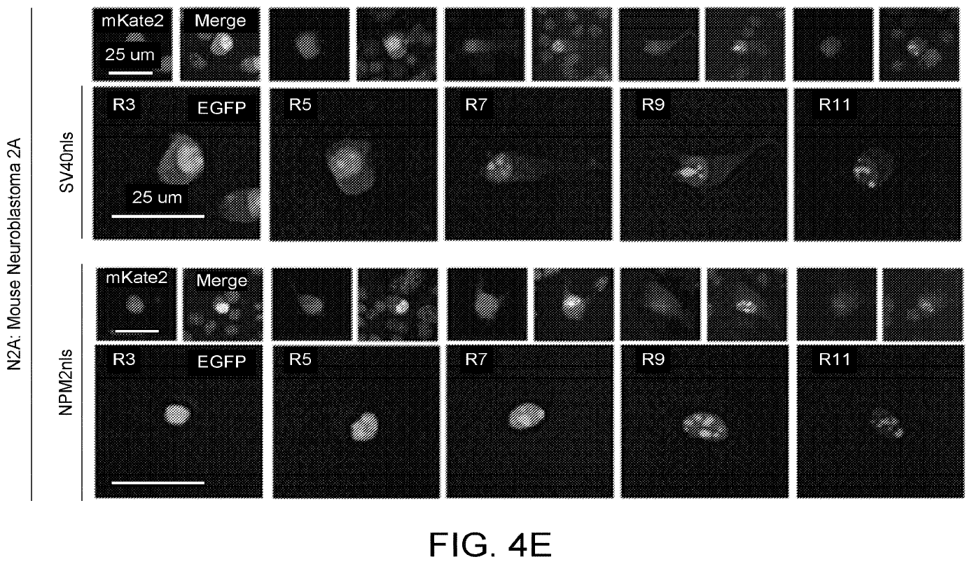

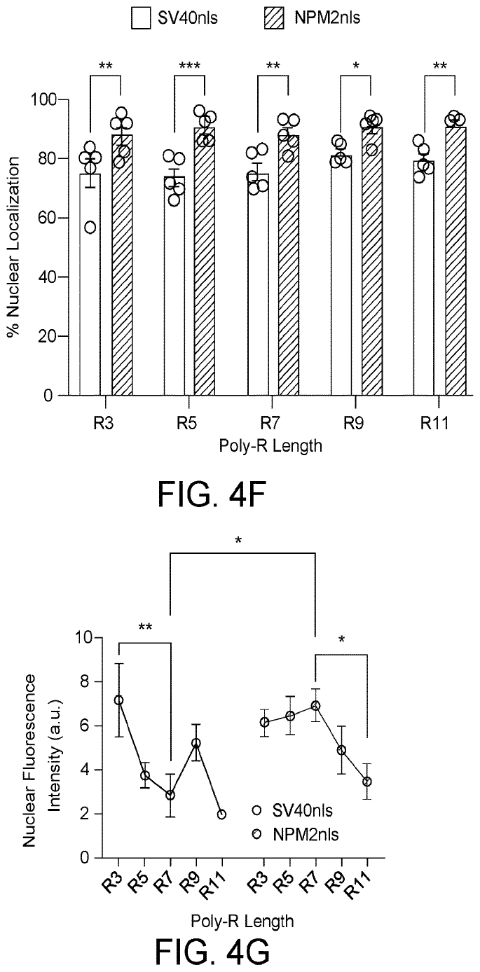

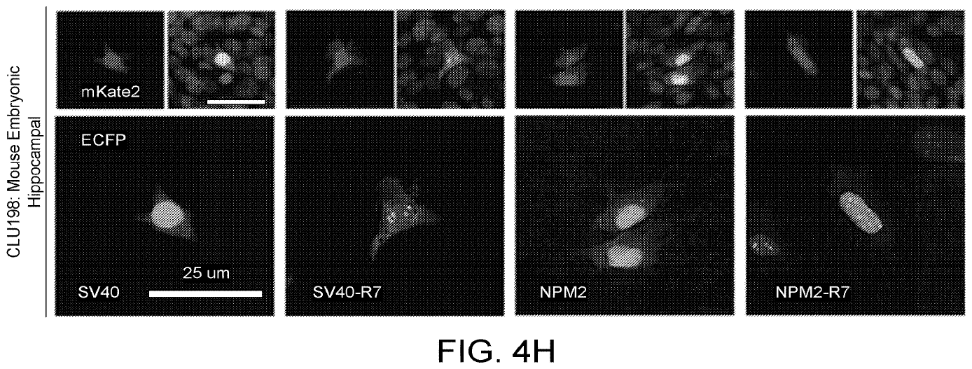

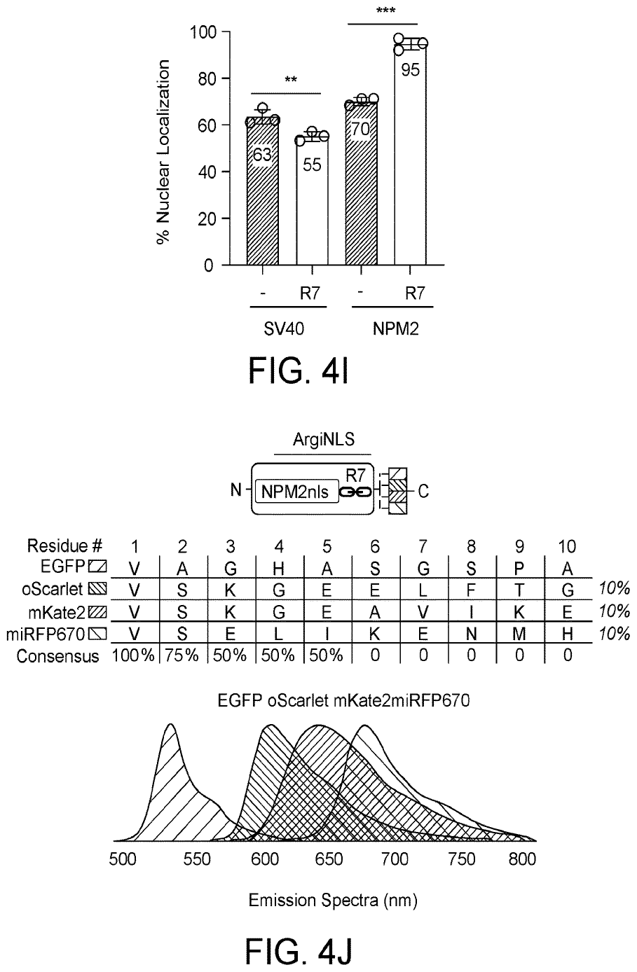

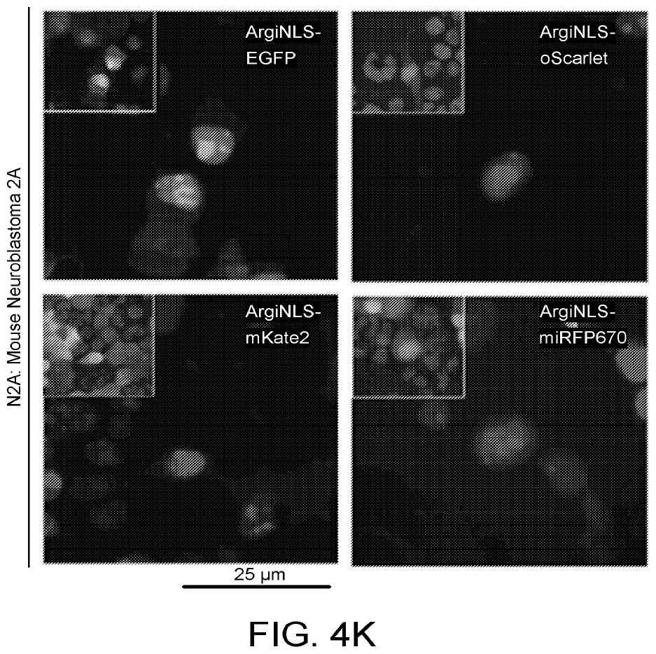

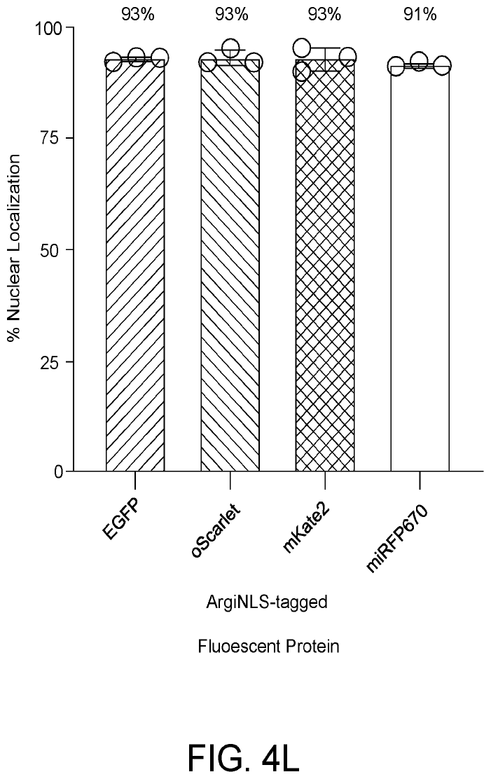

FIGS. 4A-4L illustrate the strategy and in vitro characterization of an arginine-rich NLS (ArgiNLS). FIG. 4A illustrates a schematic depicting extracellular signal caused by classical NLS (cNLS) tag use, and depicting the theoretical effects of an optimized NLS tag that eliminates extra-nuclear signal artifact through a subnuclear enrichment strategy. FIG. 4B (Top) illustrates a configuration of cNLS, poly-arginine nucleolar enrichment tag, and EGFP in AAV overexpression vectors created for in vitro testing. FIG. 4B (Bottom) illustrates corresponding amino acid composition of mono- or bipartite cNLS consensus sequences followed by poly-R stretch separating the coding sequence (CDS) of EGFP (first 10 amino acids shown). FIG. 4C includes amino acid sequences of SV40nls- and NPM2nls-poly-R tags. Basic amino acids are indicated in bold. d, Heat maps of isoelectric point (pl) and net positive charge (z) of each tag configuration. FIG. 4E provides representative images of transiently co-transfected single N2A cells displaying untagged mKate2 and SV40nls-poly-R (top) or NPM2nls-poly-R (bottom) tagged EGFP expression, spanning 3 to 11R. FIG. 4F provides percent nuclear localization of each NLS across poly-R lengths (n=5/condition). FIG. 4G illustrates nuclear fluorescence intensity (a.u.=arbitrary units) across poly-R lengths for each NLS from same cells measured in 4F. FIG. 4H shows representative images of transient co-transfections with untagged mKate2 and each experimental EGFP-expressing construct within immortalized CLU198 embryonic hippocampal mouse cell line. FIG. 4I provides percent nuclear localization with/without R7 addition for each cNLS. FIG. 4J illustrates a molecular schematic of ArgiNLS tag (NPM2nls-R7) and amino acid sequence comparison and consensus of the first 10 N-terminal residues to EGFP, oScarlet, mKate2, and miRFP670. FIG. 4K provides representative images of transient co-transfections with (from L to R and top to bottom) ArgiNLS-EGFP/mKate2, ArgiNLS-oScarlet/EGFP, ArgiNLS-mKate2/EGFP, and ArgiNLS-miRFP670/EGFP combinations within N2A mouse cell line. FIG. 4L provides percent nuclear localization of ArgiNLS-tagged FPs. FIGS. 4I and 4L include cell culture summary data that is from 5 blinded and randomly selected cells quantified per transfection condition across 3 replicate cultures. The data in 4F was statistically analyzed using a two-way ANOVA followed by Holm-Sidak's multiple comparison post hoc test. The data in 4G was statistically analyzed using a two-way ANOVA followed by Holm-Sidak's multiple comparison post hoc tests for effects of poly-R length both within and across each cNLS. The data in 4 L was statistically analyzed using a two-way (NLS(3)×+/−R7 (2)) ANOVA followed by Sidak post hoc test. *p<0.05; **p<0.01; ***p<0.005.

Design of a nuclear-restricting Arginine-rich NLS (ArgiNLS): Arginine I is the most hydrophilic, basic natural amino acid residue with a pKa over 12, and possession of a positive charge within all biological environments. Because of these properties, synthetic polyarginine (poly-R) tags (i.e., a peptide sequence consisting of multiple, R repeats) serve a variety of experimental functions, including their use as a protein purification tag(F. Bartnicki, et al., Sci Rep 7, 2619 (2017); S. M. Fuchs, R. T. Raines, Protein Sci 14, 1538-1544 (2005); S. J. Brewer, H. M. Sassenfeld, Trends in Biotechnology 3, 119-122 (1985); K. Hristova, W. C. Wimley, J Membr Biol 239, 49-56 (2011)), cell-penetrating peptide signal, multi-DNA-binding domain linker, and nucleolar trafficking signal(F. Bartnicki, et al., Sci Rep 7, 2619 (2017); R. M. Martin, et al., Nucleus 6, 314-325 (2015); N. Schmidt, et al., FEBS Letters 584, 1806-1813 (2010); M. Imanishi, et al., Biochemical and Biophysical Research Communications 333, 167-173 (2005)). It was determined whether a poly-R stretch coded in-between an NLS sequence and a downstream FP could act as an improved nuclear-localizing agent within neurons (FIG. 4A).

To explore this idea in vitro, the effects of a range of poly-R lengths on the nuclear localization of two known classical NLS (cNLS) types were tested: (i) monopartite (MP), composed of a single cluster of 4-8 basic amino acids and consensus recognition motif of K (K/R) X (K/R) (X is any amino acid), and (ii) bipartite (BP), composed of two clusters of 2-3 positively charged amino acids, separated by a 9-12 amino acid linker region, and with a consensus sequence R/K(X)₁₀₋₁₂KRXX(B. Cautain, et al, FEBS J 282, 445-462 (2015); N. Freitas, C. Cunha, Curr Genomics 10, 550-557 (2009)) (FIG. 4B; Table 2). cNLS sequences derived from the widely applied and characterized MP SV40(R. E. Lanford, J. S. Butel, Cell 37, 801-813 (1984); N. Ishii, et al., Journal of Virology 70, 1317 (1996)) or BP nucleoplasmin2 (NPM2) (C. Dingwall, et al., Cell 30, 449-458 (1982)) genes were selected, whose NLS protein sequences differ in length and net positive charge but share the same isoelectric point (pl) across poly-R length additions (FIGS. 4C, 4D). Co-transfections of AAV-(SV40nls or NPM2nls)-poly-R-EGFP overexpression constructs were performed with untagged mKate2 in mouse neuroblastoma 2A (N2A) cells. Nuclear or cellular fluorescence localization and brightness (see Methods for a detailed description quantification approach) in individual EGFP+/mKate2+ cells were measured using DAPI staining as a nuclear marker and untagged mKate2 expression to label entire cells.

TABLE 2
Sequence information of NLS tags tested.

| | Origin | | | | | |
|---|---|---|---|---|---|---|
| | NCBI | | | | | |
| | accession | | | | | |
| NLS tag | number | Sequence | Length | pI | Z | CDS |
| SV40 | NP_0431271 | PKKKRKV | 7 | 11.5 | +4.9 | CCTAAGAAAAAGAGGAAGGTG |
| | | (SEQ ID NO: | | | | (SEQ ID NO: 14) |
| | | 13) | | | | |
| SV40-R3 | na | PKKKRKV- | 10 | 12.8 | +7.9 | CCTAAGAAAAAGCGCAAGGTG-AGAAGGCGG |
| | | RRR (SEQ ID | | | | (SEQ ID NO: 16) |
| | | NO: 15) | | | | |
| SV40-R5 | na | PKKKRKV- | 12 | 13.1 | +9.9 | CCTAAGAAAAAGCGCAAGGTG- |
| | | RRRRR (SEQ | | | | AGAAGGCGGCGGCGC (SEQ ID NO: 18) |
| | | ID NO: 17) | | | | |
| SV40-R7 | na | PKKKRKV- | 14 | 13.3 | +11.9 | CCTAAGAAAAAGCGCAAGGTG- |
| | | RRRRRRR | | | | AGAAGGCGGCGGCGCCGTCGA |
| | | (SEQ ID NO: | | | | (SEQ ID NO: 20) |
| | | 19) | | | | |
| SV40-R9 | na | PKKKRKV- | 16 | 13.4 | +13.9 | CCTAAGAAAAAGCGCAAGGTG- |
| | | RRRRRRRRR | | | | AGAAGGCGGCGGCGCCTCGAAGGAGA (SEQ ID |
| | | (SEQ ID NO: | | | | NO: 22) |
| | | 21) | | | | |
| SV40-R11 | na | PKKKRKV- | 18 | 13.5 | +15.9 | CCTAAGAAAAAGCGCAAGGTG- |
| | | RRRRRRRRR | | | | AGAAGGCGGCGGCGCCGTCGAAGGAGACGTCG |
| | | RR (SEQ ID | | | | C (SEQ ID NO: 24) |
| | | NO: 23) | | | | |
| biSV40 | NP_0431271 | KRTADGSEFE | 19 | 9.9 | +2.1 | AAAAGAACCGCCGACGGCTCCGAATTCGAGAGC |
| | | SPKKKRKV | | | | CCTAAGAAAAAGCGCAAGGTG |
| | | (SEQ ID NO: | | | | (SEQ ID NO: 25) |
| | | 4 | | | | |
| biSV40- | na | KRTADGSEFE | 26 | 12.5 | +9.1 | AAACGCACCGCCGACGGCAGCGAGTTTGAGTCT |
| R7 | | SPKKKRKV- | | | | CCCAAGAAAAAGCGCAAAGTAGAA- |
| | | RRRRRRR | | | | AGGAGAAGAAGGAGAAGGAGGGGG (SEQ |
| | | (SEQ ID NO: | | | | ID NO: 26) |
| | | 3) | | | | |
| NPM2 | NP_ | KRVAPQKQM | 17 | 11.6 | +6.1 | AAAAGGGTGGCTCCCCAGAAGCAGATGAGCATA |
| | 001398932.1 | SIAKKKKV | | | | GCAAAGAAAAAGAAGGTG (SEQ ID NO: 27) |
| | | (SEQ ID NO: | | | | |
| | | 2) | | | | |
| NPM2-R3 | na | KRVAPQKQM | 20 | 12.8 | +9.1 | AAAAGGGTGGCTCCCCAGAAGCAGATGAGCATA |
| | | SIAKKKKV- | | | | GCAAAGAAAAAGAAGGTG-AGAAGGCGG |
| | | RRR (SEQ ID | | | | (SEQ ID NO: 29) |
| | | NO: 28) | | | | |
| NPM2-R5 | na | KRVAPQKQM | 22 | 13.1 | +11.1 | AAAAGGGTGGGTCCCCAGAAGCAGATGAGCATA |
| | | SIAKKKKV- | | | | GCAAAGAAAAAGAAGGTG-AGAAGGCGGCGGCG |
| | | RRRRR (SEQ | | | | (SEQ ID NO: 31) |
| | | ID NO: 30) | | | | |
| NPM2-R7 | na | KRVAPQKQM | 24 | 13.3 | +13.1 | AAAAGGGTGGCTCCCCAGAAGCAGATGAGCATA |
| | | SIAKKKKV- | | | | GCAAAGAAAAAGAAGGTG- |
| | | RRRRRRR | | | | AGAAGGCGGCGGCGCCGTCGA (SEQ ID NO: |
| | | (SEQ ID NO: | | | | 32) |
| | | 1) | | | | |
| NPM2-R9 | na | KRVAPQKQM | 26 | 13.4 | +15.1 | AAAAGGGTGGCTCCCCAGAAGCAGATGAGCATA |
| | | SIAKKKKV- | | | | GCAAAGAAAAAGAAGGTG- |
| | | RRRRRRRRR | | | | AGAAGGCGGCGGCGCCGTCGAAGGAGA (SEQ ID |
| | | (SEQ ID NO: | | | | NO: 34) |
| | | 33) | | | | |
| NPM2- | na | KRVAPQKQM | 28 | 13.5 | +17.1 | AAAAGGGTGGCTCCCCAGAAGGAGATGAGCATA |
| R11 | | SIAKKKKV- | | | | GCAAAGAAAAAGAAGGTG- |
| | | RRRRRRRRR | | | | AGAAGGCGGCGGCGCCGTCGAAGGAGACGTCG |
| | | RR (SEQ ID | | | | C (SEQ ID NO: 36) |
| | | NO: 35) | | | | |

Table 2 provides the sequences of the synthetic NLS tags used in this example. The polyarginine linker sequences are separated from the NLS sequences by a dash.

Co-transfected EGFP+/mKate+cells revealed a poly-R length-dependent increase in nucleolar localization for both cNLSs. NPM2nls, and not SV40nls, exhibited decreased extra-nuclear fluorescence regardless of poly-R length (FIG. 4E). Measured composite values across poly-R lengths was 77% (±1.48) for SV40nls and 91% (±0.85) for NPM2nls (data not shown), where NPM2nls significantly obtained stable, higher nuclear localization across all poly-R lengths (FIG. 4F). Additionally, unlike the linear decrease observed with SV40nls, nuclear brightness with NPM2nls peaked with poly-R7 and decayed with longer lengths (FIG. 4G).

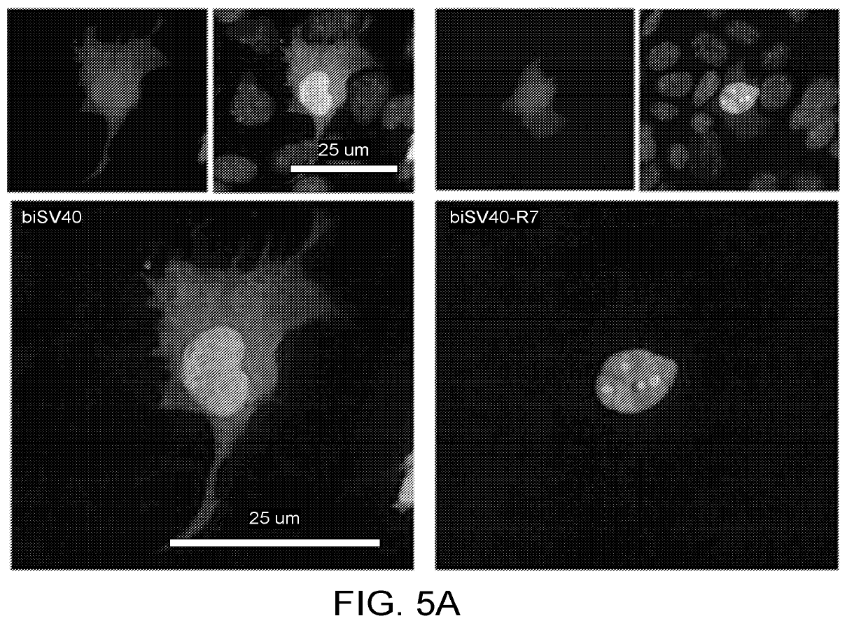

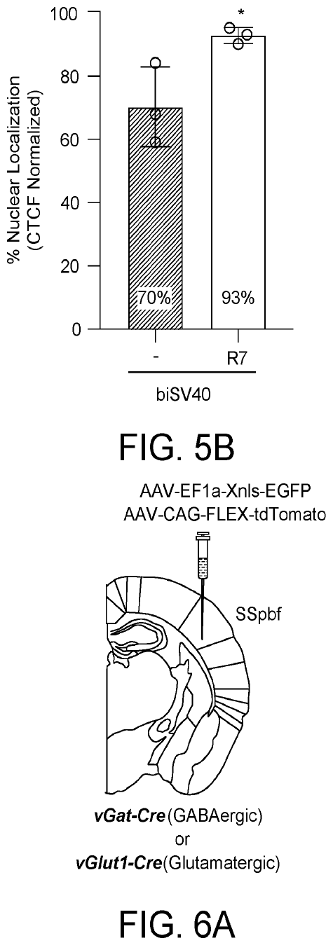

FIGS. 5A, 51B illustrate that poly-R7 synergizes bipartite SV40 NLS activity in vitro. FIG. 5A provides representative images of transient co-transfections with untagged mKate2 and biSV40- or biSV40-R7-EGFP-expressing constructs in an immortalized CLU198 embryonic hippocampal mouse cell line. FIG. 51B illustrates normalized corrected total cellular fluorescence (CTCF) of poly-R7 addition with biSV40nls (n=mean of 5 cells/3 cultures). Student's t-test. **p<0.01.

The optimal NPM2nls-R7 configuration was validated in a separate heterologous mouse brain cell-line by comparing untagged performance for each cNLS type (FIGS. 4H, 4I). The control SV40nls tag exhibited 63% of nuclear EGFP fluorescence that was significantly decreased to 55% with the poly-R7 addition, despite a nucleolar-like localization (FIGS. 4H, 4I). NPM2nls-R7 enhanced nuclear localization above untagged levels from 70 to 95% (FIG. 4I). Next, the specificity of poly-R7 on BP cNLS activity was probed using a BP version of SV40nls (biSV40nls, FIGS. 5A, 5B). An enhancement of nuclear-localized EGFP fluorescence was observed with biSV40nls-R7 (93%±1.5) versus untagged biSV40nls (70%±7.3) (FIGS. 5A, 5B). The bipartite SV40 NLS sequence is KRTADGSEFESPKKKRKV (SEQ ID NO: 4), and the bipartite SV40 NLS sequence with the polyarginine stretch is KRTADGSEFESPKKKRKVRRRRRRR (SEQ ID NO: 3).

Prior work has shown that a synthetic poly-R8 tag facilitates DNA-binding protein function when inserted as a linker sequence (N. Schmidt, et al., FEBS Letters 584, 1806-1813 (2010)), INDICATING that poly-R's can be used as a linker in other molecular contexts. Next, the ability of the NPM2nls-R7 genetic tag—now termed ArgiNLS—to provide modularity with other FPs beyond EGFP was tested (FIGS. 4J-4L). ArgiNLS ws attached to a range of spectrally separate FPs that have diverging N-terminal sequences located within the first 10 amino acids, which could influence fused NLS tag activity (FIG. 4J). Monomeric FPs included EGFP (Ex/Em: 488/507), oScarlet(53) (Ex/Em: 569/594), mKate2(54) (Ex/Em: 588/633), and miRFP670(55) (Ex/Em: 647/670) (FIG. 4J; bottom). In N2A cells, nuclear restriction of native fluorescence for EGFP (93%±0.3), oScarlet (93%±1.0), mKate2 (93%±1.5), and miRFP670 (91%±0.3) was measured (FIGS. 4K, 4L).

Nuclear restriction of FP fluorescence in vivo: The in vitro results show enhanced ArgiNLS restriction of nuclear fluorescence. Next, it was determined if the performance replicated in vivo using AAV overexpression in the mouse brain. Nuclear import of NLS-containing proteins—including SV40 and NPM2—are controlled by nuclear transport receptors called importins (or karyopherins)(N. Freitas, C. Cunha, Curr Genomics 10, 550-557 (2009)). Importins are dynamically expressed in multiple isoforms across cell-types and brain regions (K. Hosokawa, et al., Neuroscience 157, 864-877 (2008)), providing a potential cause of variable NLS activity of NLSs in a cell-type and brain region-dependent manner. A conditional AAV co-expression strategy was used to quantify nuclear-localized EGFP fluorescence in two genetically distinct cortical cell classes, GABAergic or glutamatergic neurons. It was determined that each class comprises a divergent set of multiple neuronal cell sub-types that have functionally and genetically distinct characteristics(K. S. Matho, et al., Nature 598, 182-187 (2021); Z. J. Huang, A. Paul, Nat Rev Neurosci 20, 563-572 (2019)), and therefore are well suited for benchmarking the nuclear localizing efficacy of ArgiNLS- or conventional SV40nls-tagged EGFP.

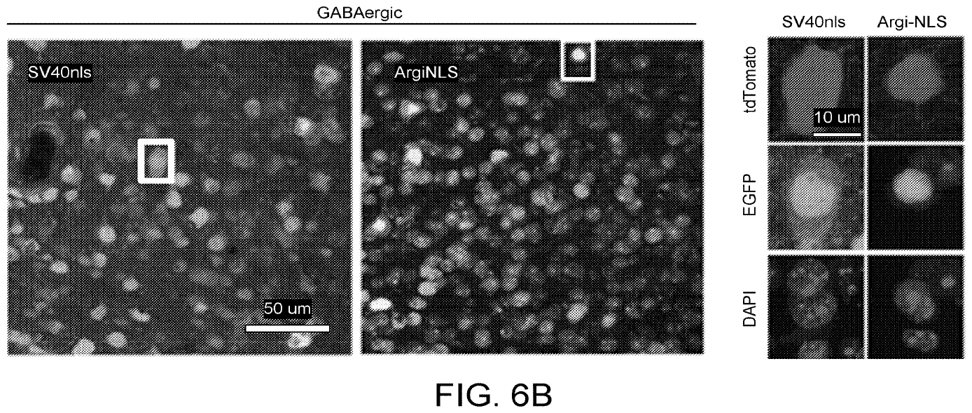

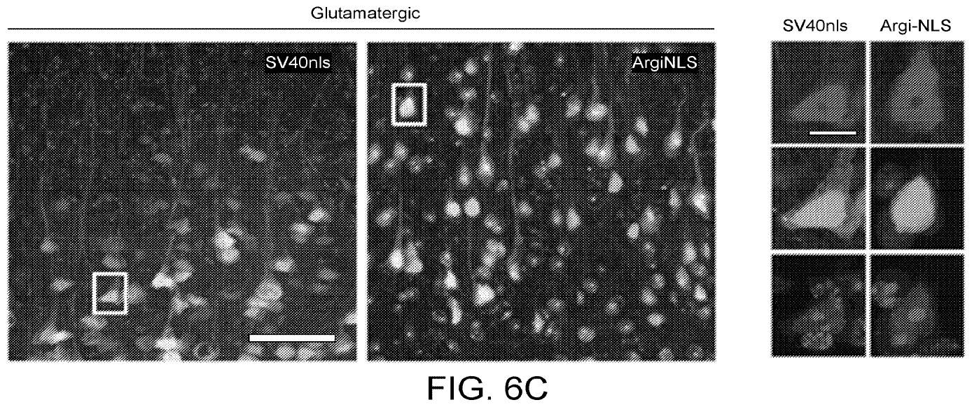

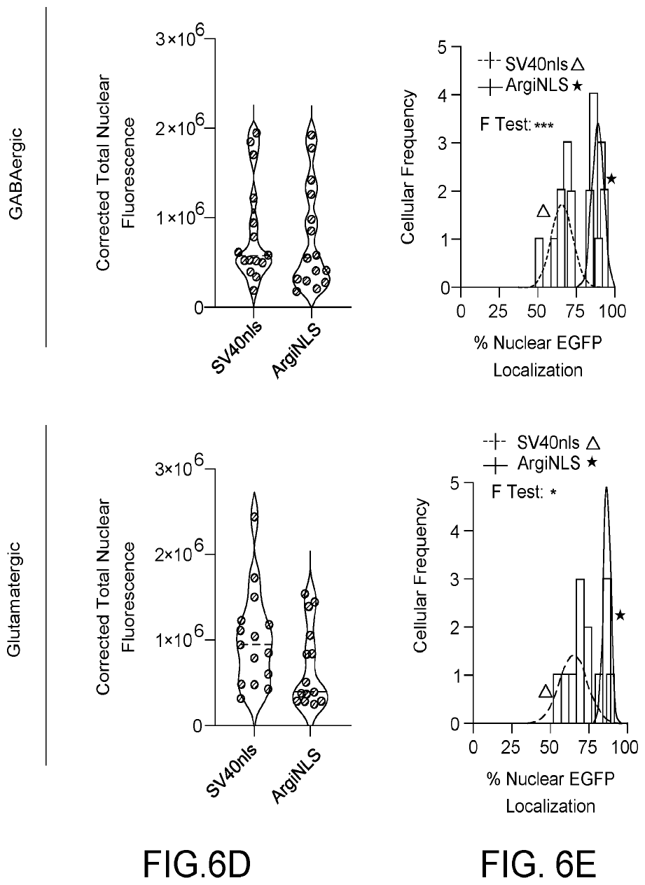

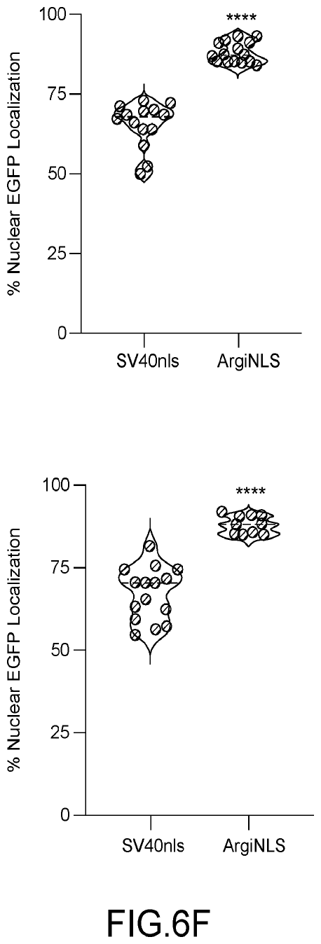

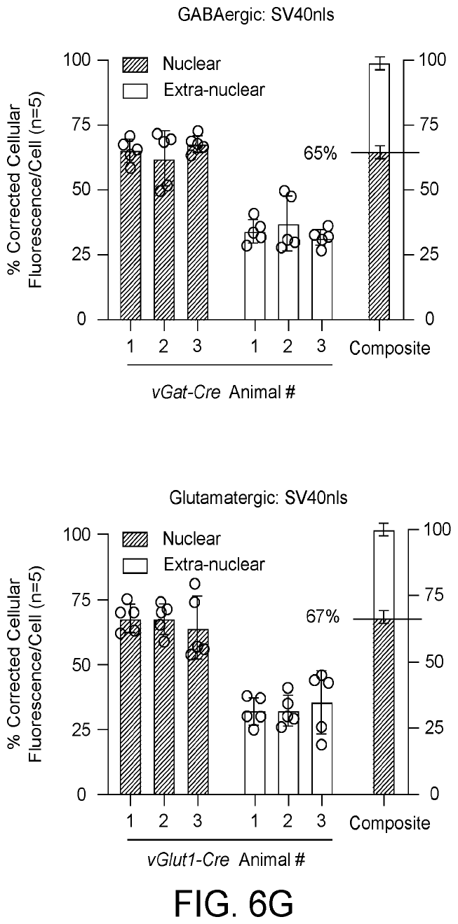

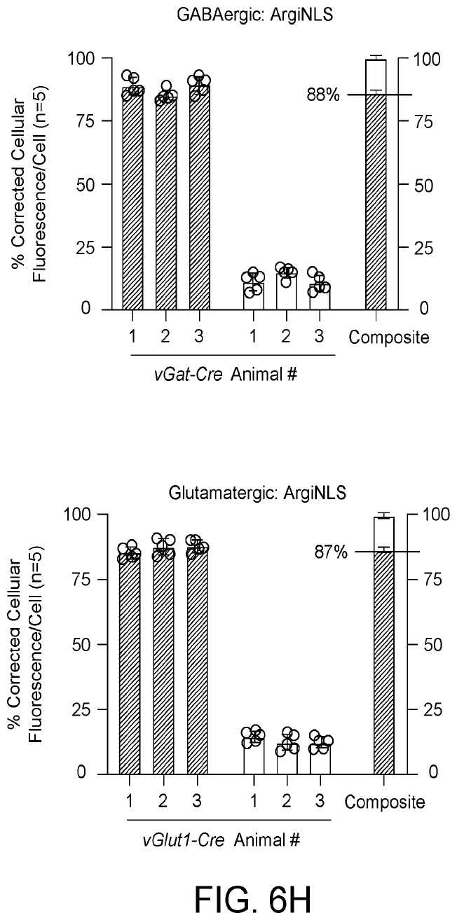

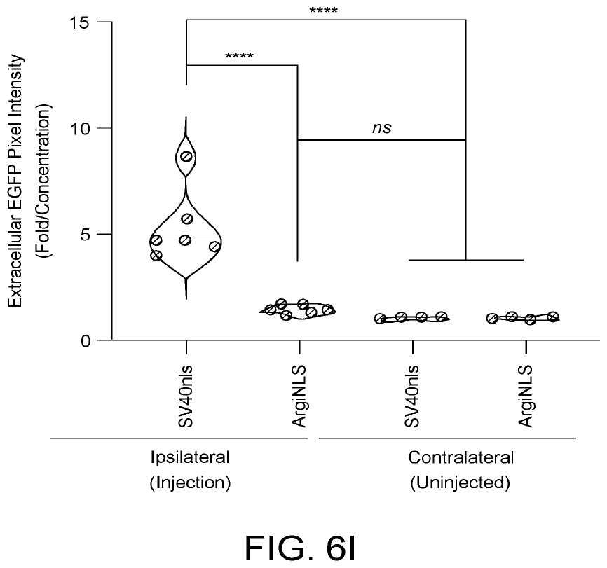

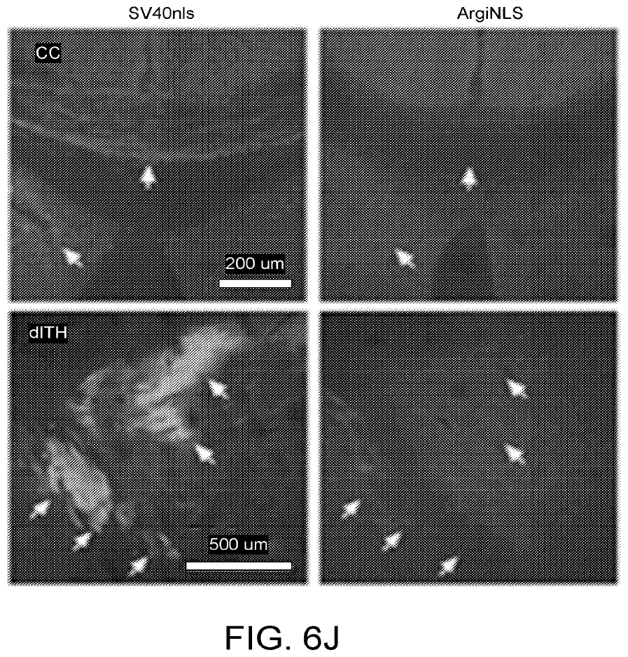

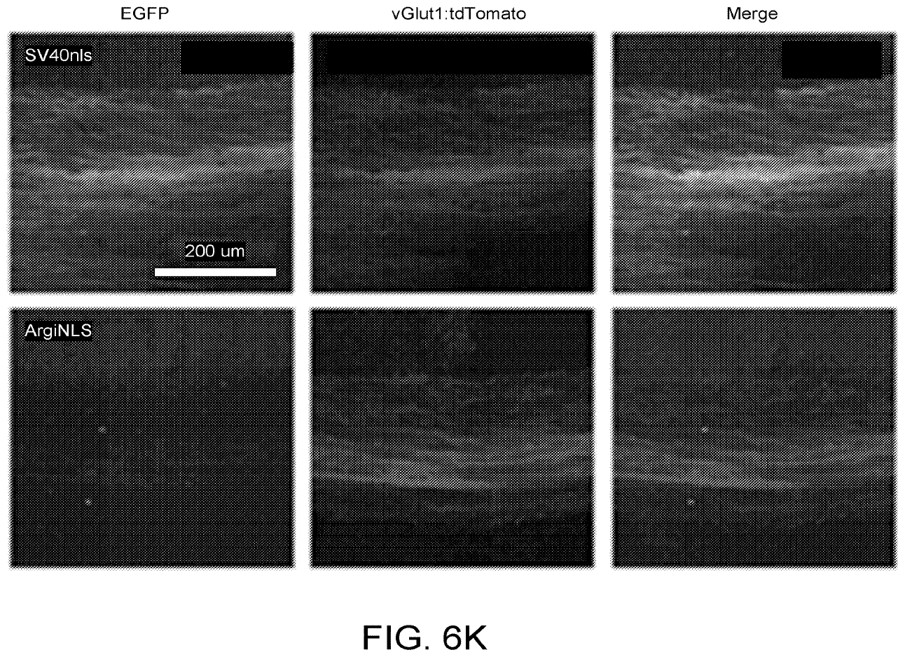

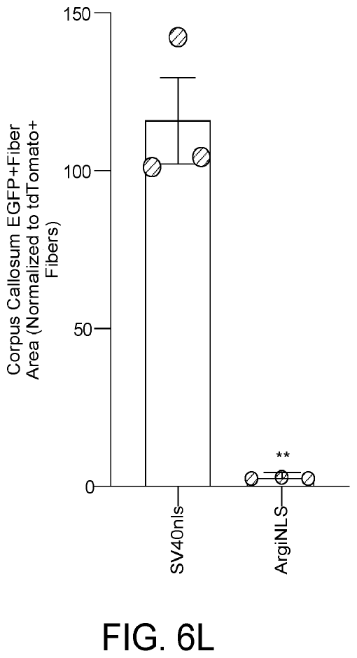

FIGS. 6A-6L illustrate optimized nuclear localization of fluorescence across major cortical cell-type classes in vivo. FIG. 6A provides a schematic of stereotaxic AAV co-injection in the barrel field of primary somatosensory cortex (SSpBF) of vGat-Cre or vGlut1-Cre transgenic mice. In FIGS. 6B, 6C, the left panel provides representative composite 40× confocal images of SV40nls-EGFP/FLEX-tdTomato and ArgiNLS-EGFP/FLEX-tdTomato viral expression in (6B) vGat-Cre or (6C) vGlut1-Cre mice. In FIGS. 6B, 6C, the right panel provides cropped representative image of an example EGFP+/tdTomato+cell displayed across separate red (tdTomato), green (EGFP), and blue (DAPI) channels.

FIGS. 6D-6H provide quantification of NLS tag performance in GABAergic (top) or glutamatergic (bottom) EGFP+/tdTomato+cells (n=random 5 cells/animal; n=3 animals per Cre-driver/tag). FIG. 6D illustrates EGFP corrected total nuclear fluorescence (CTNF). FIG. 6E illustrates cellular frequency histogram of percent nuclear EGFP localization. FIG. 6F provides percent nuclear EGFP fluorescence comparison across tags. FIGS. 6G and 6H illustrate nuclear and extra-nuclear EGFP fluorescence for all quantified cells of each animal across (6G) SV40nls- or (6H) ArgiNLS-labeled cells. The composite percent nuclear EGFP across all animals is indicated to the right. FIG. 6I illustrates total pixel intensity of extracellular (EC) EGFP signal ipsilateral to injection site, expressed as a fold change from non-injected contralateral side (n=3 EC quantifications/3 vGlut1-Cre mice). FIG. 6J provides representative images of extranuclear EGFP signal in axon bundles of passage in the corpus callosum (CC; top) or terminations in the dorsal thalamus (dTH; bottom). Arrows indicate location of labeling observed with SV40nls and are superimposed on ArgiNLS-EGFP. FIG. 6K provides representative 40× confocal images of SV40nls- or ArgiNLS-EGFP with vGlut1:tdTomato viral expression in fibers of the CC (n=3 EC quantifications/3 vGlut1-Cre mice). FIG. 6L illustrates quantification of EGFP+fiber area in CC normalized to vGlut1:tdTomato+fiber area. All bar graphs represent mean±SEM. The data in FIGS. 6D, 6F, and 6L was statistically analyzed using an unpaired t-test. Group differences in population variance of data in 6E were statistically compared using an F-test. The data in 6I was statistically analyzed using a 1-way ANOVA followed by Tukey's multiple comparison post hoc test. Violin plots display median and interquartile range as dashed lines. *p<0.01; **p<0.05; **p<0.01; ****p<0.001

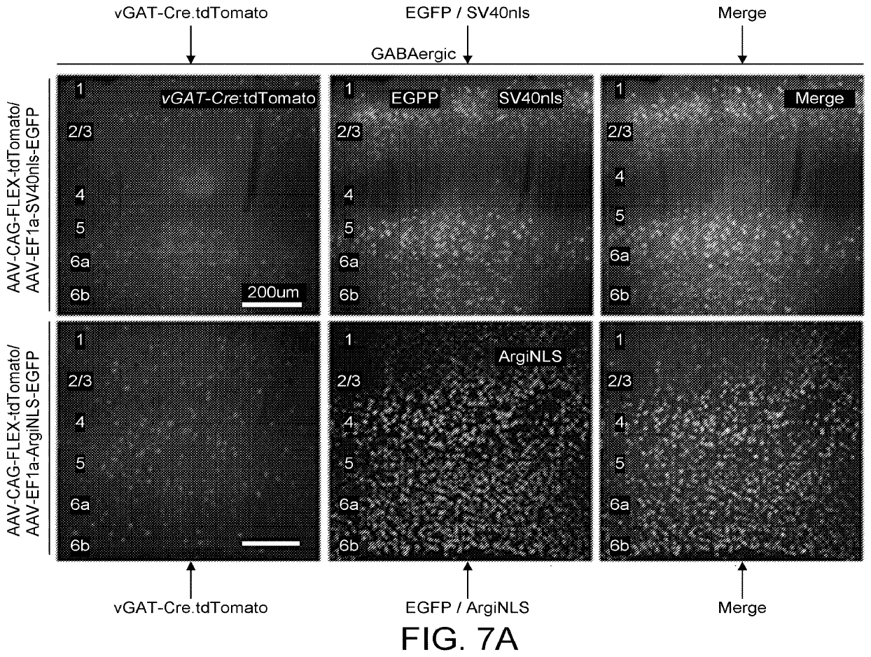

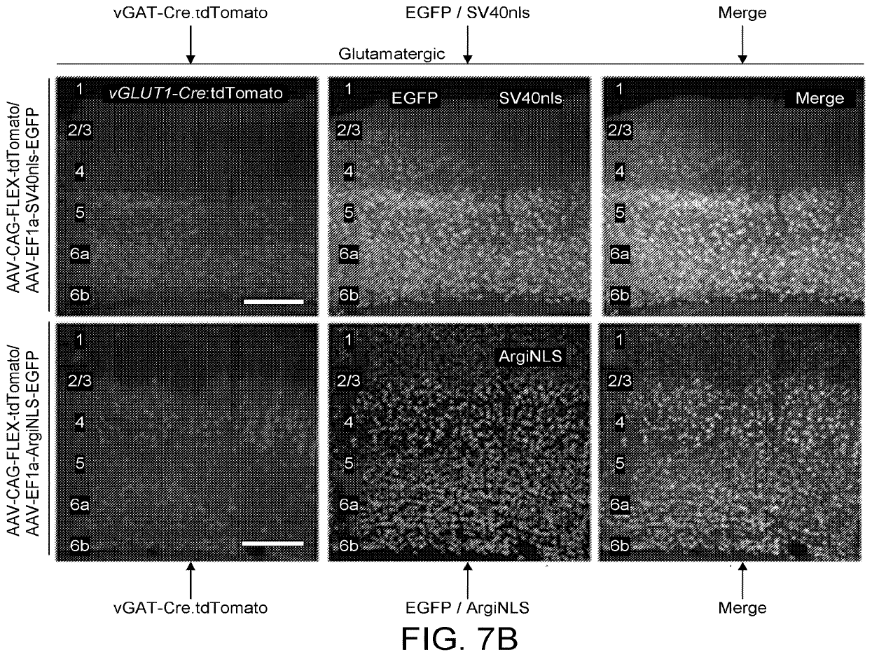

FIGS. 7A, 7B provide representative images of viral expression from local barrel cortex co-injections. Example viral expression observed in a (7A) vGAT-Cre or (7B) vGlut1-Cre animal for Cre-dependent tdTomato and SV40nls-(top row) or ArgiNLS-EGFP (bottom row) viruses.

The barrel cortex—where GABAergic and glutamatergic cell-types are abundant—was selected for AAV co-injections using SV40nls or ArgiNLS tagged EGFP (AAV-EF1α-SV40nls- or ArgiNLS-EGFP), combined with a conditional Cre-dependent virus to express somatic tdTomato (AAV-CAG-FLEX-tdTomato) in GABAergic or glutamatergic cells within vGat-Cre or vGlut1-Cre animals, respectively (FIG. 6A) (K. S. Matho, supra; Z. J. Huang, A. Paul, supra). Confocal images were acquired of the injection site, and nuclear (as defined by DAPI+co-stain) and somatic (as defined by tdTomato+somatic fluorescence) EGFP fluorescence was quantified at the level of single-cells across NLS tags and cell-types at both high (FIGS. 6B, 6C) and low (FIGS. 7A, 7B) magnification.

Barrel cortex neurons infected with SV40nls-EGFP exhibited somatic EGFP concomitant with high extracellular background, putatively from SV40nls-EGFP localized in cellular processes (FIGS. 6B, 6C; left). Conversely, ArgiNLS-EGFP did not exhibit extra-cellular EGFP background, which corresponded with nuclear-restricted EGFP and localization in presumptive nucleolar puncta (FIGS. 6B, 6C; right). Sampled EGFP+/tdTomato+single-cells used for nuclear/somatic EGFP fluorescence quantifications contained equal distributions of nuclear localization (corrected total nuclear fluorescence, CTNF; see Methods) levels across tags and cell-type (FIG. 6D). However, when CTNF was expressed as a percent cellular fluorescence (corrected total cellular fluorescence, CTCF; see Methods), nuclear localization variance was significantly different across tags; SV40nls showed high variance while ArgiNLS showed low variance (FIG. 6E). Despite differences in variance, tag type had a significant effect on nuclear localization, where ArgiNLS-EGFP contained significantly higher nuclear localization (GABAergic cells: 88%±0.9; glutamatergic cells: 87%±0.7) than SV40nls-EGFP (GABAergic cells: 65%±1.8; glutamatergic cells: 67%±2.0) (FIGS. 6F-6H).

Next, it was determined how ArgiNLS impacts extracellular (EC) background fluorescence at the site of injection and within anatomically distal areas (FIGS. 6I-6L). First, EC pixel intensity measurements taken from field of views (FOVs) positioned in-between EGFP-labeled cells of the injected barrel cortex (i.e., ipsilateral), and in-between DAPI+cells of the uninjected (i.e., contralateral) barrel cortex, as a control measurement, from the same mice were compared (FIG. 6I). Ipsilateral SV40nls FOVs contained a significant 4-fold increase in EC fluorescence compared to uninjected contralateral FOVs, that was also significantly higher than between-subject ipsilateral and contralateral ArgiNLS FOVs. EC fluorescence within ipsilateral ArgiNLS FOVs was unchanged from contralateral FOV levels from both SV40nls and ArgiNLS injected samples.

Neurons within the barrel cortex send diverse axonal projections throughout the brain(l. M. Zakiewicz, et al, Front Neuroinform 8, 5 (2014)). ArgiNLS consistently eliminated non-nuclear labeling of axon fiber of passage in the corpus callosum (CC), as well as axonal terminations in the dorsolateral thalamus (dITH), that was observed with SV40nls-EGFP injections (FIG. 6J). Decreased CC axon labeling by ArgiNLS was confirmed in separate quantifications made of EGFP sampled in high-magnification FOVs. To control for error in sample-to-sample variability of viral delivery, the analysis was restricted within the vGlut1-Cre injected sections and normalized EGFP+fiber area to vGlut1:tdTomato+fiber area (FIGS. 6K, 6L). Accordingly, SV40nls-EGFP labeled 115% (±13) of vGlut1:tdTomato+fibers, indicating that extra-nuclear EGFP localization produced from constitutively expressed SV40nls-EGFP labels CC fibers within both vGlut1(+) and vGlut1(−) neurons of the barrel cortex (FIG. 6L). Conversely, ArgiNLS-EGFP labeled 3% (±1.2) of vGlut1:tdTomato+fibers.

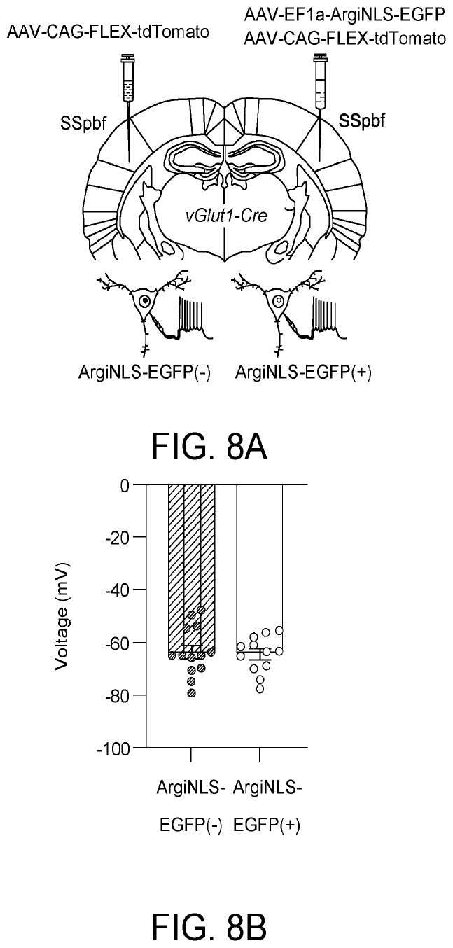

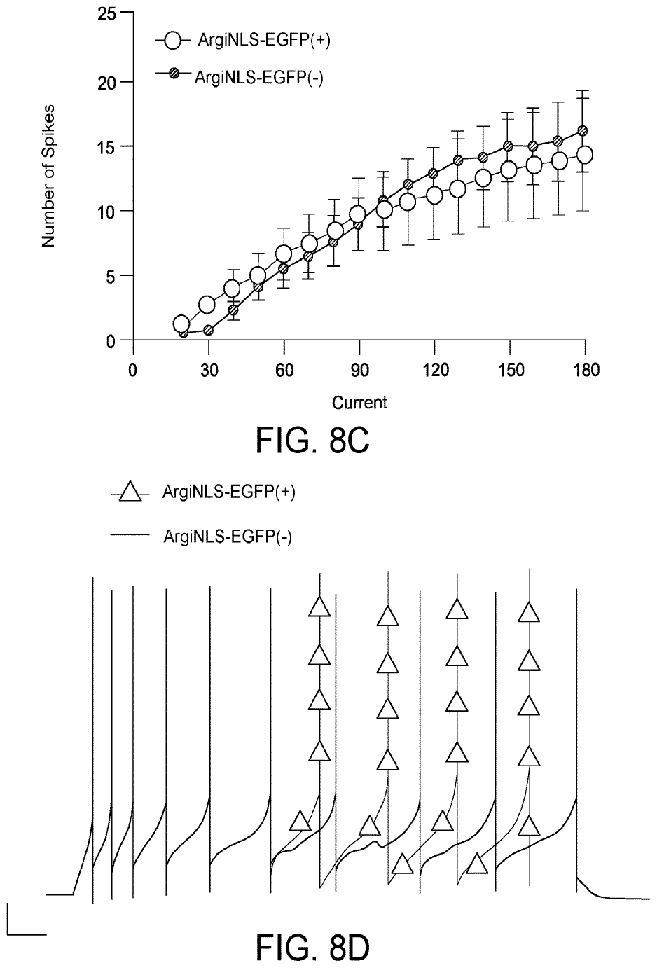

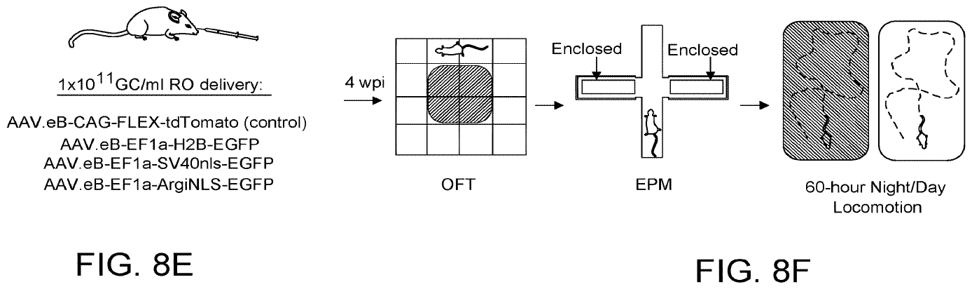

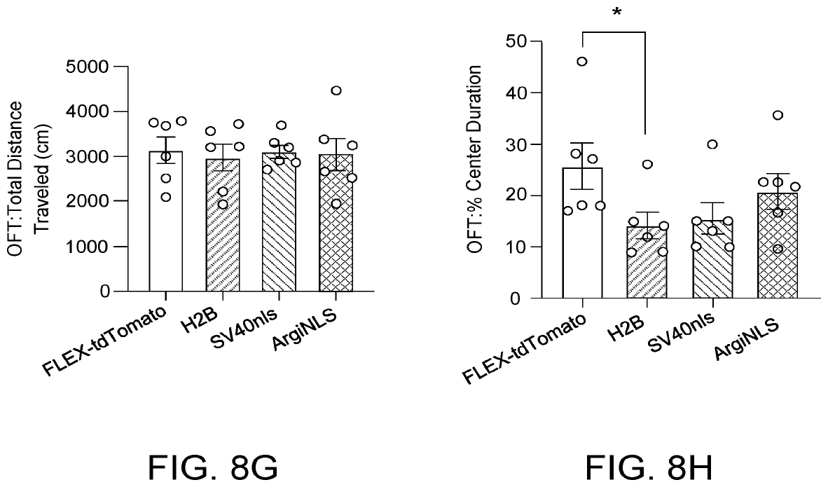

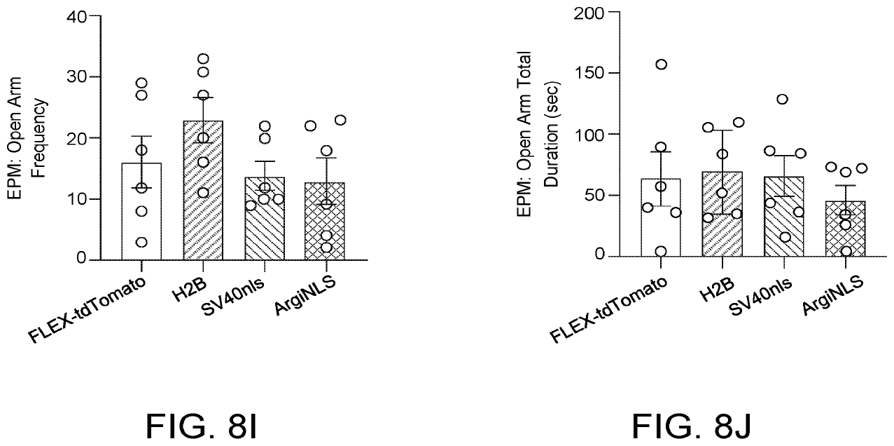

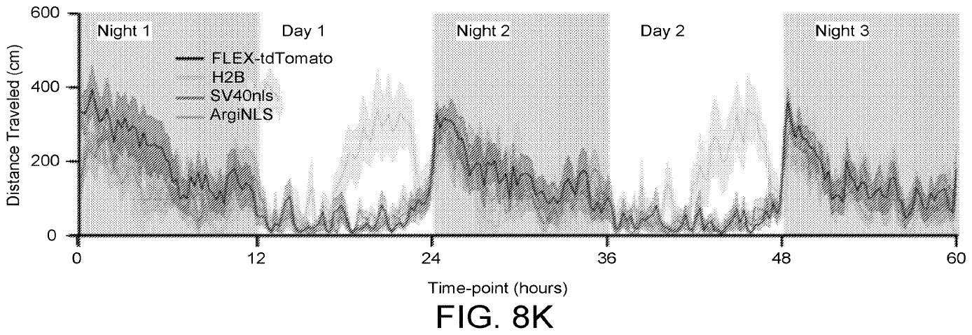

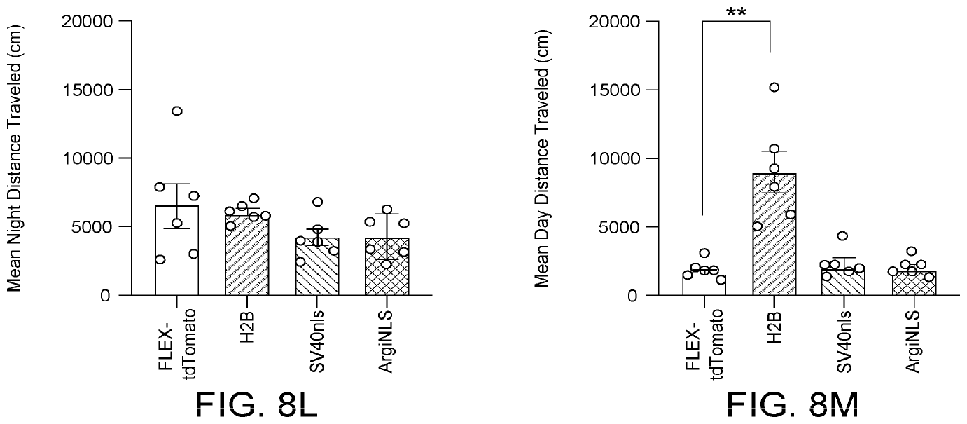

FIGS. 8A-8M illustrate that ArgiNLS-EGFP viral expression is not associated with physiological or behavioral deficits. FIG. 8A provides a schematic illustration of bilateral stereotaxic AAV injection/co-injection in barrel cortices (SSpBF) of vGlut1-Cre transgenic mice, and electrophysiological recordings conducted on resulting vGlut1-Cre:tdTomato(+) neurons that are either ArgiNLS-EGFP(−) or ArgiNLS-EGFP(+). FIG. 8B illustrates resting membrane potential of ArgiNLS-EGFP(−) (n=13) or ArgiNLS-EGFP(+) (n=12) neurons. FIG. 8C illustrates current injection curve from ArgiNLS-EGFP(−) or ArgiNLS-EGFP(+) neurons. FIG. 8D illustrates example trace current injection at 100 pA. FIG. 8E provides an AAV.eB delivery and vector information schematic. Groups of mice were injected with 1×10¹¹ GC of a negative control AAV.eB-CAG-FLEX-tdTomato (n=11), AAV.eB-EF1α-H2B-EGFP (n=8), AAV.eB-EF1α-SV40nls-EGFP (n=11), or AAV.eB-EF1α-ArgiNLS-EGFP (n=11). FIG. 8F provides baseline behavioral battery 4 weeks post viral injection (wpi). Mice were sequentially tested in the open field test (OFT), elevated plus maze (EPM), and 60-hour night/dark locomotion recording. FIG. 8G illustrates total distance traveled in the OFT. FIG. 8H provides percent time duration spent in the center of the OFT arena. FIG. 8I provides a frequency of entries in the open arm of the EPM. FIG. 8J illustrates the total duration of time spent in the open arm of the EPM. FIG. 8K provides alocomotion XY plot showing total distance traveled across time of the 60-h recording. Day (yellow)/night (gray) phase of light cycle is underlaid the plot according to time of 60-h recording. FIG. 8L illustrates mean night distance traveled across the 60-h recording period. FIG. 8M illustrates mean day distance traveled across the 60-h recording period. All bar graphs represent mean±SEM. The data for each panel from 8G-8J, 8L, and 8M were statistically analyzed using an one-way ANOVA with Dunnet's multiple comparison posthoc test of each experimental group versus control FLEX-tdTomato group. ****p<0.0001.

Non-deleterious to neuronal and behavioral health: Full-length nuclear protein-FP fusions, such as H2B-EGFP (T. Kanda, et al, Curr Biol 8, 377-385 (1998)), demonstrate high levels of nuclear-restricted fluorescence, but cause detrimental effects to cellular and animal health when overexpressed in the mouse brain (S. Ito, et al., Nat Commun 5, 4450 (2014)). Since ArgiNLS tagged FPs display similar nuclear-restricted fluorescence, it was next determined if ArgiNLS-EGFP viral overexpression in the brain causes detrimental effects to baseline neuronal physiology or animal behavior (despite it lacking the fusion of an endogenous nuclear protein) (FIGS. 8A-8M). Baseline physiological properties of glutamatergic neurons within the barrel cortex of vGlut1-Cre animals were first examined. A local unilateral injection of Cre-dependent tdTomato virus was used to create control vGlut1-Cre:tdTomato(+) labeled neurons, and a contralateral co-injection of Cre-dependent tdTomato with ArgiNLS-EGFP viruses to create within-sample, experimental ArgiNLS-EGFP(+)/vGlut1-Cre:tdTomato(+) labeled neurons (FIG. 8A). Analysis of baseline physiological parameters indicated no effect of ArgiNLS-EGFP overexpression on resting membrane potential (FIG. 8B) and action potential generation across a range of injected currents (FIGS. 8C, 8D).

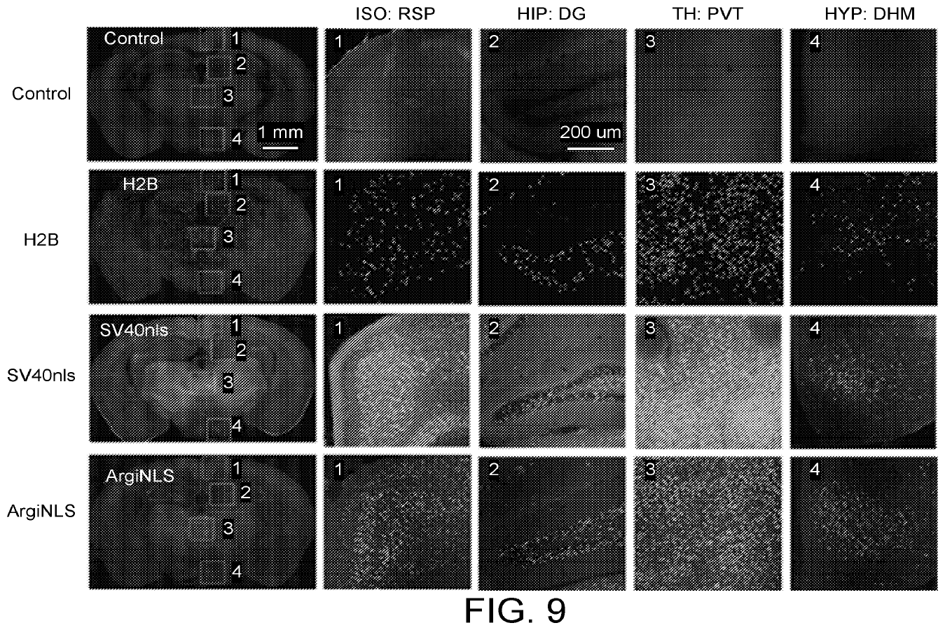

FIG. 9 illustrates representative systemic PHP.eB viral EGFP expression from behavioral cohort. Left: Example montage of coronal brain slices (−1.94 Bregma) from representative samples from each group. Numbered inset FOVs are magnified to the right. Acronyms: HIP=hippocampus; DG=dentate gyrus; TH=thalamus; PVT=paraventricular thalamus; HYP=hypothalamus; DMH=dorsomedial hypothalamus; ISO=isocortex; RSP=retrosplenial cortex.

Next, PHP.eB viral serotype was used to examine the effect of systemic whole-brain ArgiNLS-EGFP or control virus expression—including a non-expressing negative Cre-dependent tdTomato control virus, and positive SV40nls-EGFP and H2B-EGFP expressing controls—on behaviors including the open field task (OFT) and elevated plus maze (EPM) to study effects on acute exploratory drive, and a 60-hour locomotion monitoring assay to study chronic locomotion and sensorimotor performance across dark/light cycles (FIGS. 8G-8M). No differences were found in acute OFT performance across viral groups as measured by mean total distance (cm) traveled (FIG. 8G), or percent center time duration (FIG. 8H). Additionally, acute EPM performance as measured by open arm entry frequency (FIG. 8I), or open arm total duration (sec) (FIG. 8J) was the same across viral groups. Consecutive monitoring of homecage distance traveled (cm) across 3 nights and 2 days indicated a lack of effect from ArgiNLS-EGFP or SV40nls-EGFP systemic overexpression (FIGS. 8K-8M). Conversely, H2B-EGFP-injected animals displayed dysregulated locomotor activity during the light phase. It was confirmed that animals used for behavioral phenotyping contained robust systemic expression in the brain by performing 2D histology and imaging in a subset of animals (FIG. 9).

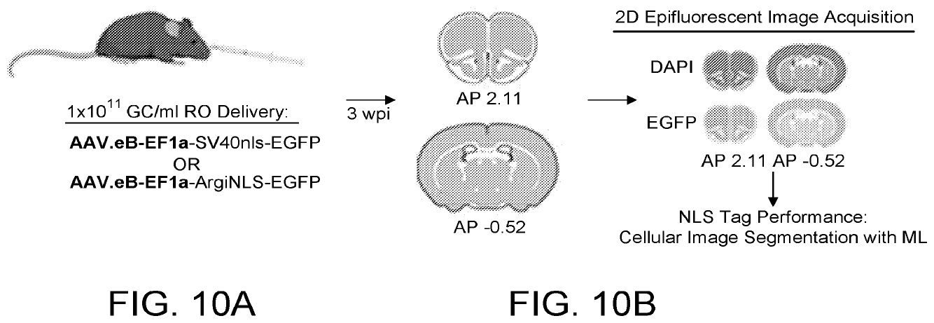

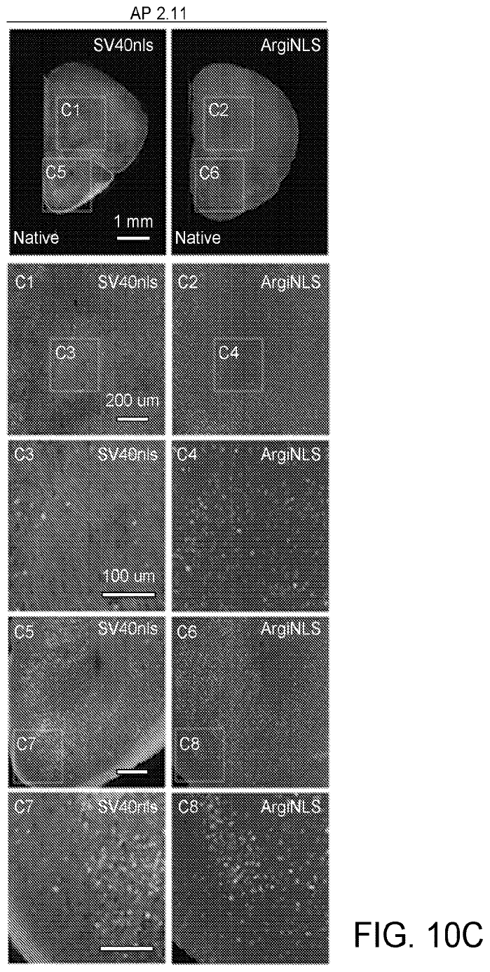

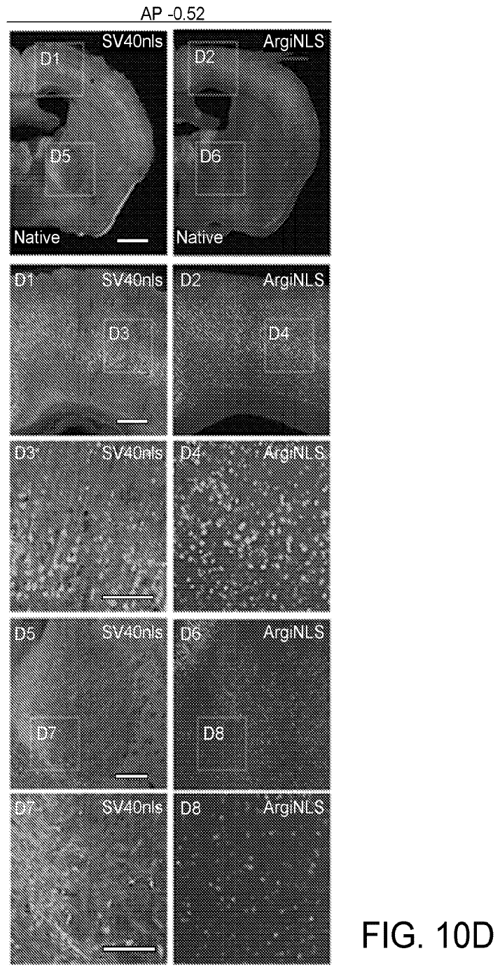

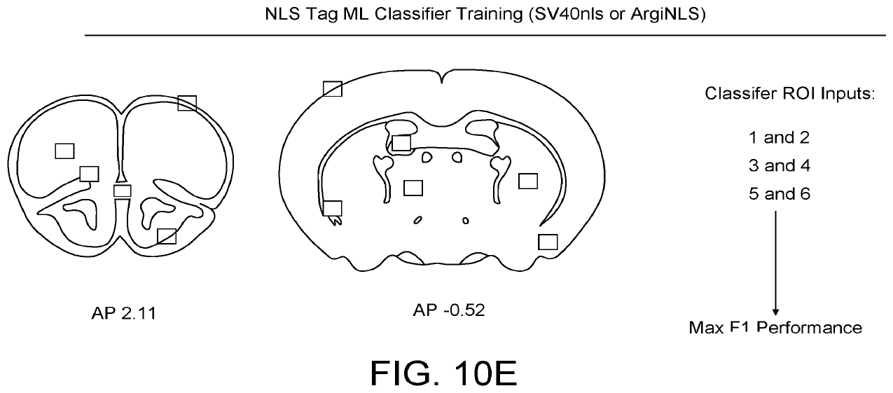

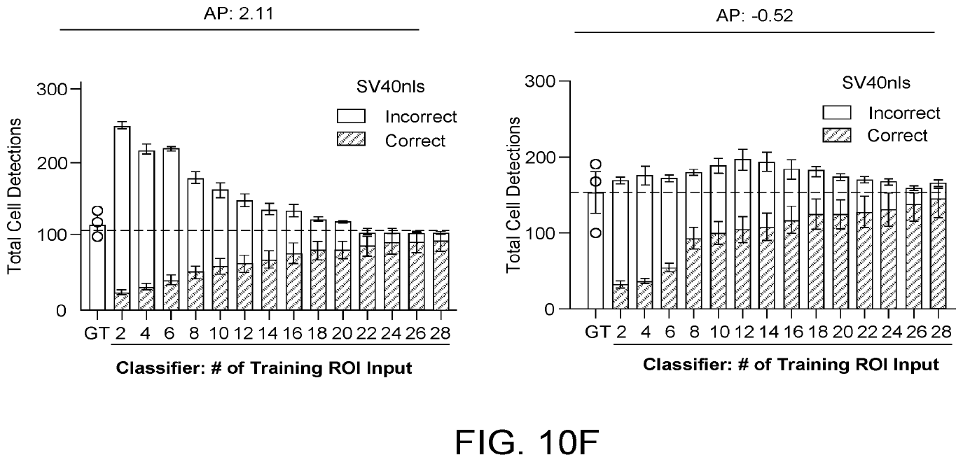

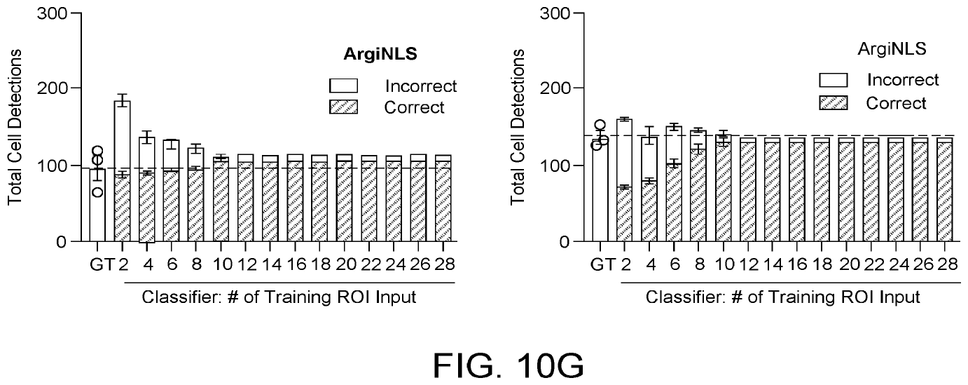

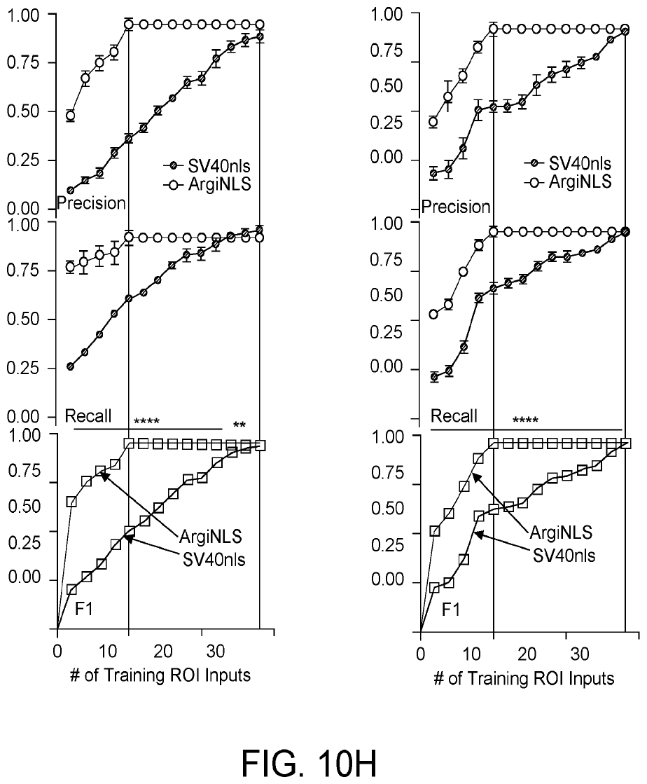

FIGS. 10A-10H illustrate enhanced performance of single-cell ML classification. FIG. 10A provides an experimental schematic of systemic AAV.eB delivery of NLS-tagged EGFP. FIG. 10B provides a microscopy acquisition schematic. FIGS. 10C, 10D provides representative images of EGFP viral expression at matching (10C) AP 2.11 or (10D) −0.52 hemispheric coronal planes, across 2 FOVs at 3 magnification levels. FIG. 10E provides a schematic of SV40nls and ArgiNLS classifier training workflow for single-cell segmentation of 2D images. FIGS. 10F-10H illustrates cell segmentation classifier benchmarking and performance metrics for (left) AP: 2.11 and (right) AP: −0.52 brain sections. Total cell detections for (6F) SV40nls-EGFP or (6G) ArgiNLS-EGFP for expert rater ground truth (GT; left bar) (n=3) and ML classifier across iterations of 2-increment additive training input rounds. Gray bars represent iterations after peak performance criteria was met. Dashed line indicates mean GT cell detections. FIG. 10H illustrates precision, recall, and F1 harmonic scoring of single-cell detection for SV40nls-EGFP and ArgiNLS-EGFP. Gray data points represent iterations after max F1 performance was met. All bar graphs represent mean±SEM. The F1 data in 10H was statistically analyzed using a two-way repeated measures ANOVA followed by Holm-Sidak's post hoc test. *p<0.05; **p<0.01; **p<0.01; ****p<0.001.

Enhanced compatibility with machine learning classification: Image classification of objects can be reliably automated and scaled through the creation of machine learning (ML) algorithms or “classifiers” that rapidly extract features based on pixel content to create image segmentations for quantification (C. Stringer, et al., Nat Methods 18, 100-106 (2021); O. Ronneberger, et al., U-Net. arXiv.org (2015)). For fluorescently labeled images, the manual effort taken to compile training datasets is directly tied to the fluorescent signal's fidelity to label a target class. Thus, poorly defined labeling may require extensive training and user annotation. Since ArgiNLS enables consistent FP restriction in the nucleus of neurons, it was next determined if this influences the creation—or performance—of a single-cell ML classifier, as compared to a classifier trained with cSV40nls tag labeling (FIGS. 10A-10H).

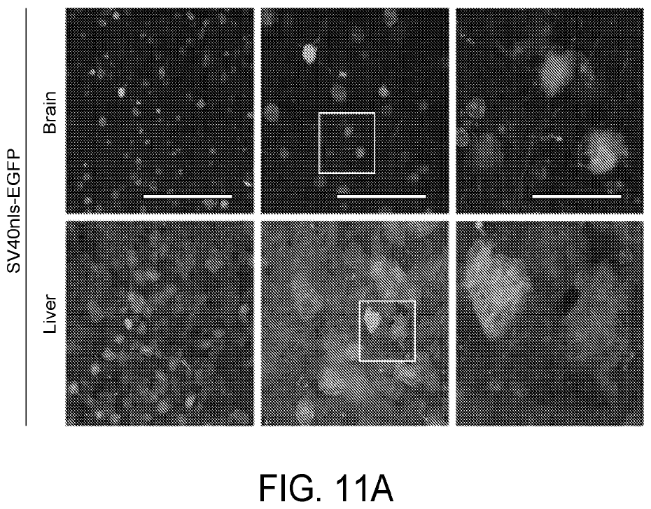

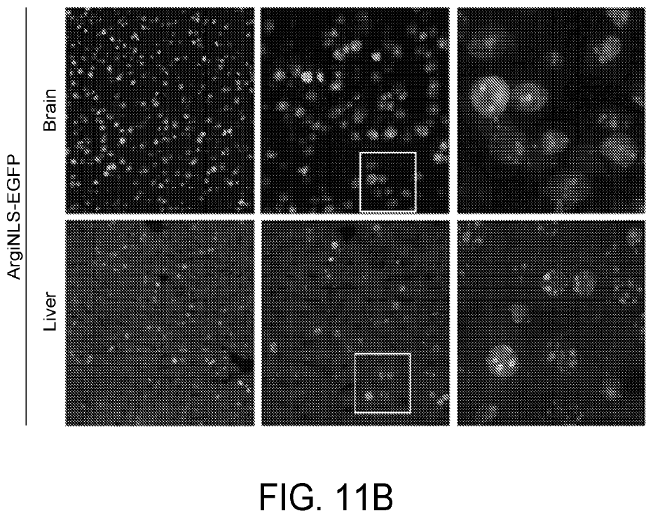

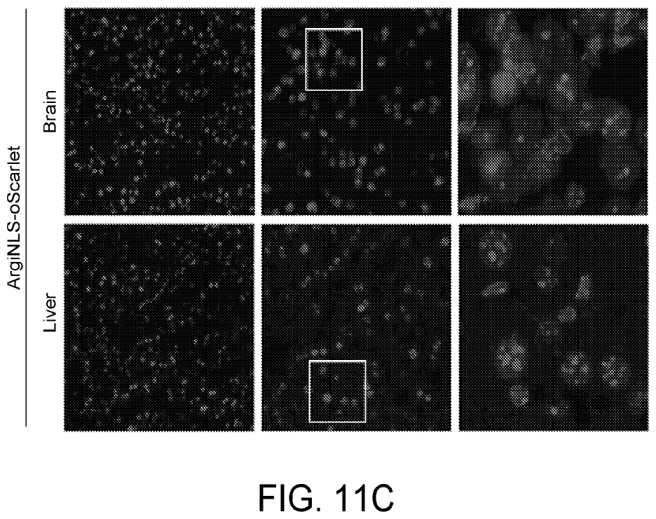

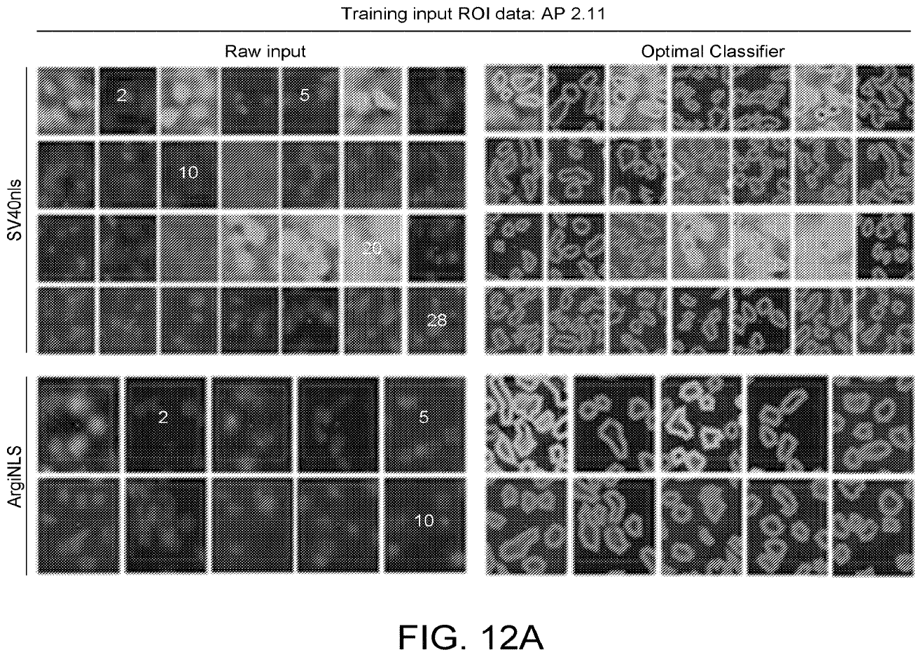

FIGS. 11A-11C illustrate representative tagged FP localization in brain and peripheral liver tissue of single animals infected with systemic Php.eB AAV. Confocal images displaying native fluorescence of (11A) SV40nls-EGFP, (11B) ArgiNLS-EGFP, and (11C) ArgiNLS-oScarlet in brain (barrel cortex) and liver tissue from single animals infected with 3×10¹¹ GC of Php.eB virus. Scale bars: left=200 um; center=100 um; right=25 um.

Single-cells were systemically labeled with SV40nls-EGFP or ArgiNLS-EGFP-expressing AAV.eB viruses through RO injections (FIG. 10A) and used DAPI counterstained images of coronal sections (2.11 and −0.52 from bregma) to benchmark single-cell ML classifier training and testing at varying anatomical locations throughout the brain (FIG. 10B). It was observed that SV40nls displayed variable extra-nuclear fluorescence and background, and ArgiNLS conversely showed nuclear-restriction and an overall higher density of labeled single-cells (FIGS. 10C, 10D). The difference in FP localization within brain cells generalized to the liver within a separate cohort of animals systemically infected with a higher titer of virus (FIGS. 11A-11C).

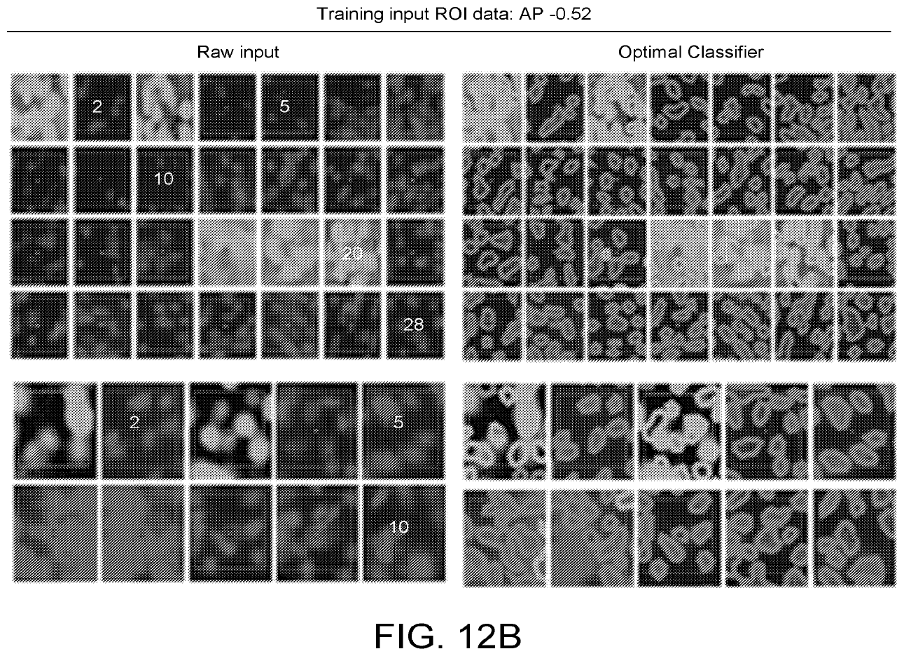

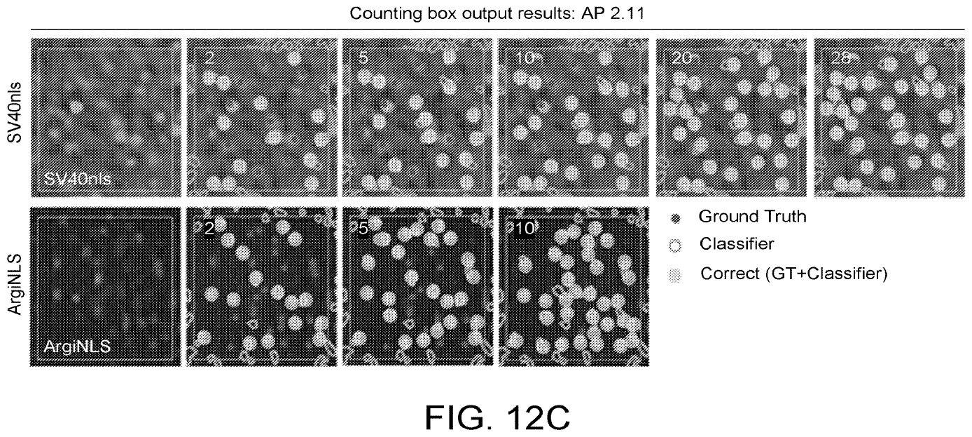

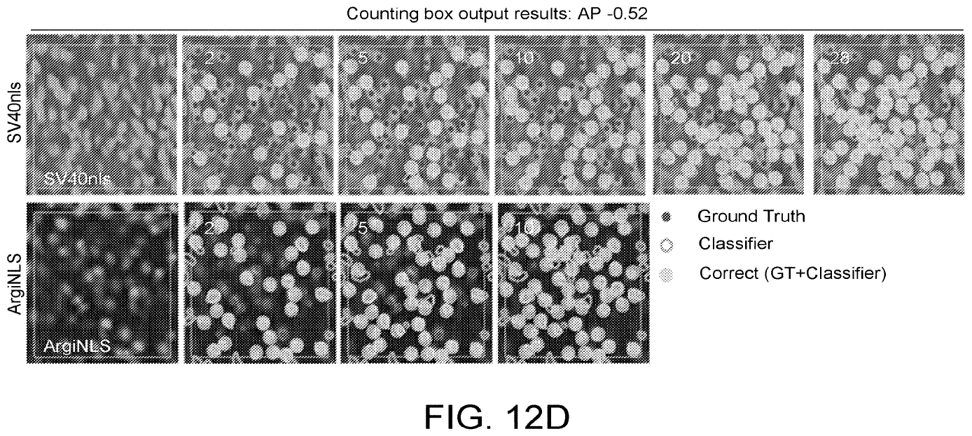

FIGS. 12A-12D provide representative 2D raw images and performance of single-cell ML classification. FIGS. 12A, 12B provide classifier training input images for SV40nls (top) of ArgiNLS (bottom) from coronal brain section position corresponding to Aps (12A) 2.11 or (12B) −0.52 from bregma. Raw input images are show to the left and the result shown to the right. The final training input image is numbered in red, white numbers indicate training input level.

FIGS. 12C, 12D provide raw images of counting box FOVs used for ground truth markup and comparisons with ML classifier at Aps (12C) 2.11 and (12D) −0.52 for SV40nls (top) and ArgiNLS (bottom). White numbers indicate amount of input images (from 12A, 12B) used for classifier training.

Single-cell classifiers were created for SV40nls and ArgiNLS labels and benchmarked their performance using F1 scores: a machine learning metric that determines model accuracy based on a combination of precision (i.e., positive predictions) and recall (i.e., correct identification of positive class) measurements. Multiple training iterations were performed consisting of two sample FOVs per session until maximum classifier performance, as measured by F1 scoring in FOVs with similar ground truth (GT) cell detections, was reached (FIGS. 10E, 12A-12D). ArgiNLS (5 rounds, 10 input FOV) resulted in a substantial reduction in the total number of training iterations required to reach max classifier performance at both anatomical planes compared to SV40nls (14 rounds, 28 input FOV) (FIGS. 10F-10H). Specifically, for AP 2.11, SV40nls classification started with a 93% greater total amount of cell detections than GT, that was composed of 11% correct (24±3.1) and 89% (222±3.8) incorrect detections. Both correct and incorrect detections displayed an exponential-like scaling towards GT levels as function of training round number (FIG. 10F; left). Initial ArgiNLS classification consisted of 63% more total cell detections (184±7.5; Correct: 87 7.0; Incorrect: 96±13.9) than GT, with correct cells matching 90% of GT (FIG. 10G; left), and incorrect cells progressively reduced over each training session.

Total cell detections for both classifiers at AP −0.52 was stable across all training iterations and corresponded closely to GT detection levels with training (FIGS. 10F, 10G; right). Like the raw data, ArgiNLS classification outperformed SV40nls classification in both precision (FIG. 10H, top) and recall (FIG. 10H, middle). ArgiNLS significantly outperformed SV40nls in F-sore across nearly all training tile input FOV iterations (FIG. 10H; bottom).

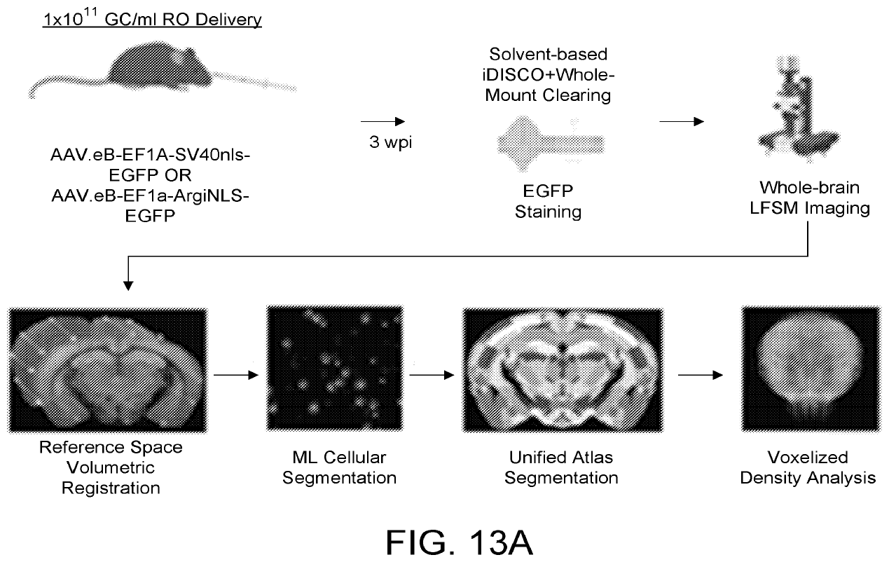

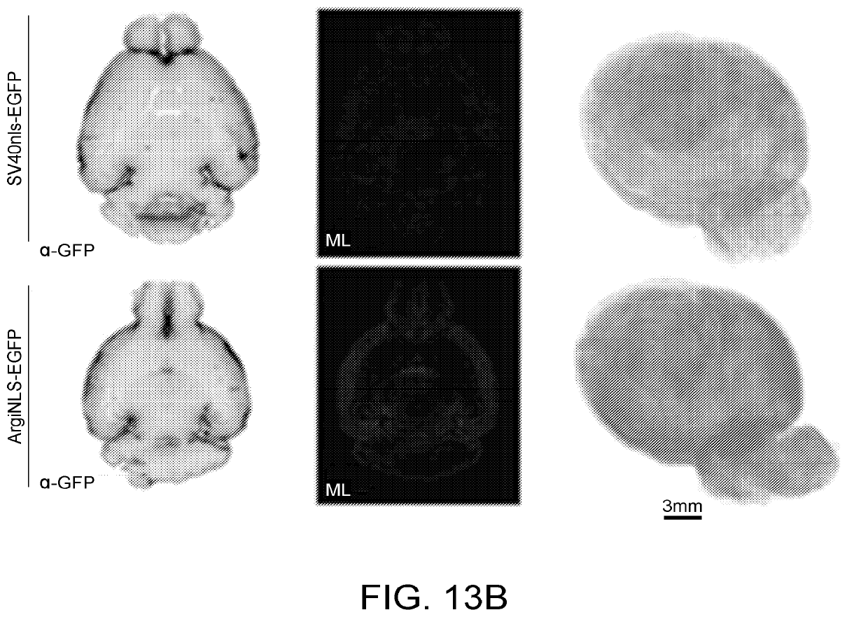

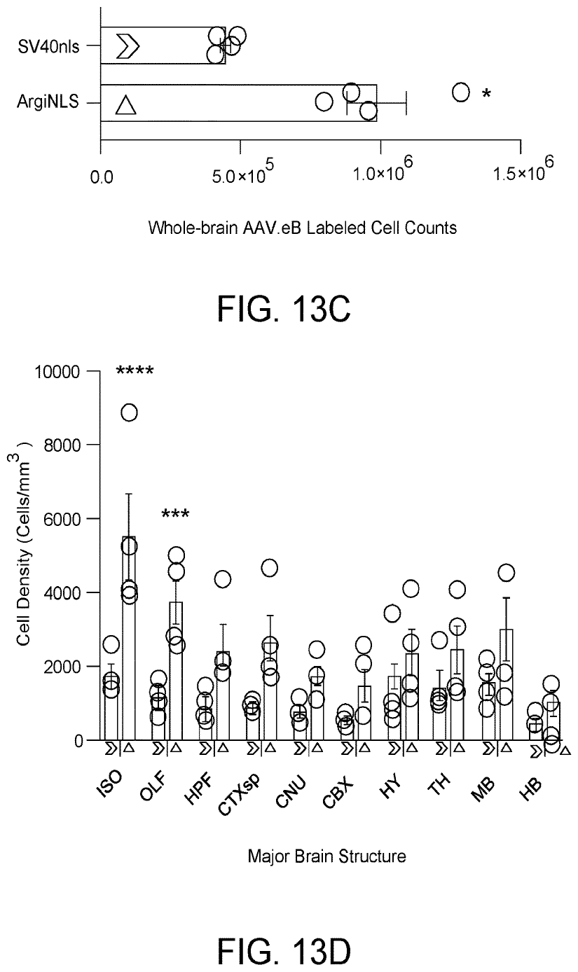

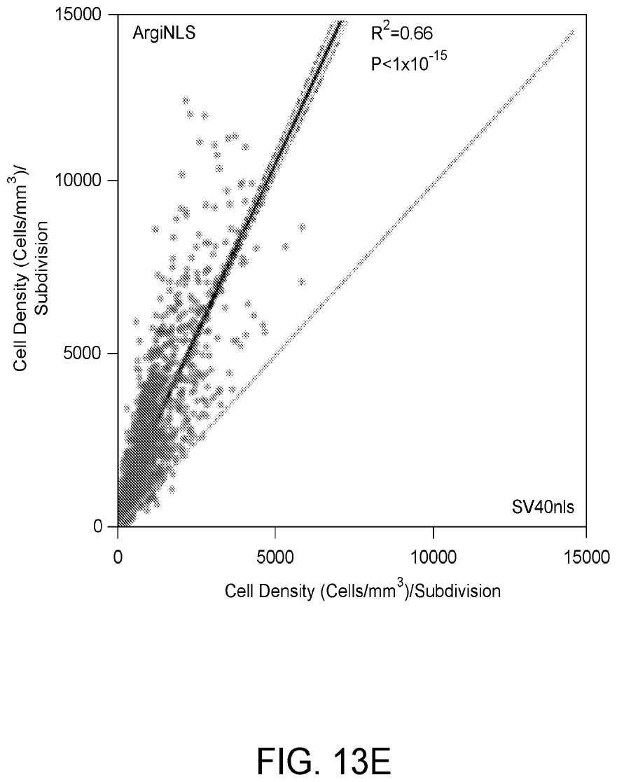

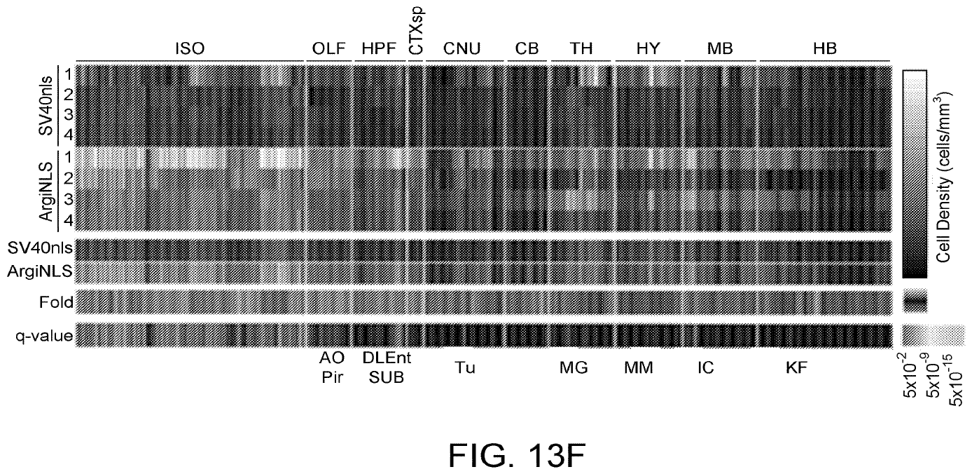

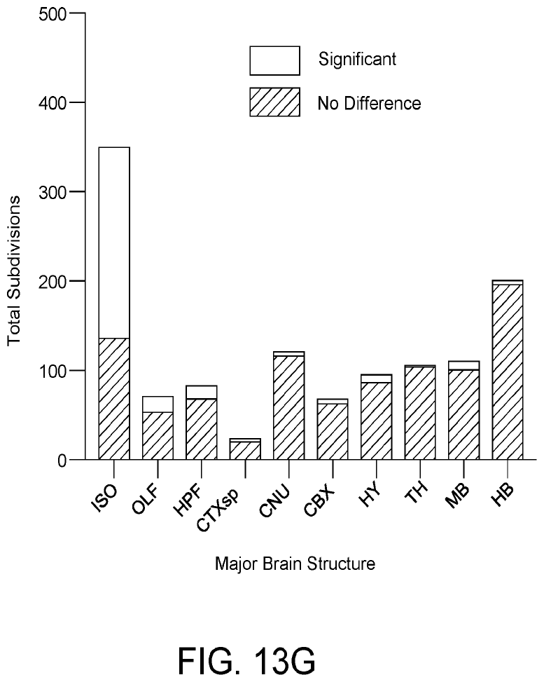

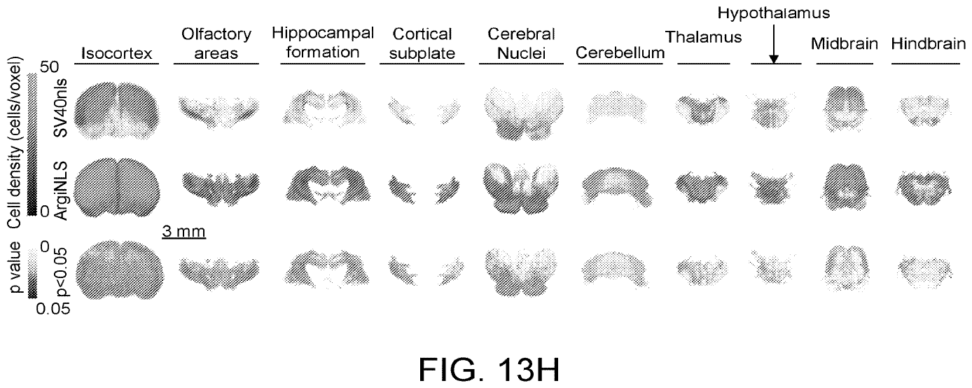

FIGS. 13A-13M illustrate enriched brain-wide segmentation of single-cells. FIG. 13A provides a schematic of experimental and image processing workflow used to quantify systemic SV40nls- or ArgiNLS-EGFP-labeled cells across the whole mouse brain. In FIG. 13B, the left panel provides a raw LSFM image in horizontal orientation from a representative SV40nls-EGFP (top) or ArgiNLS-EGFP (bottom) expressing brain sample. In FIG. 13B, the middle panel provides corresponding ML-segmented cells within the same optical section. In FIG. 13B, the right panel provides whole-brain 3D renderings of voxelized cell density for each sample. FIG. 13C illustrates a comparison of whole-brain AAV.eB-labeled cell counts. FIG. 13D illustrates a comparison of cell density across major ontological brain structures. FIG. 13E provides a scatterplot and linear regression of ArgiNLS (Y-axis) versus SV40nls (X-axis)-labeled mean cell density across all subdivisions of the brain. FIG. 13F provides a 2D heatmap plot of (from top to bottom): cell density across each individual sample, mean cell density, fold change over ArgiNLS, and statistical significance of mean cell density differences across all anatomical subdivisions by structure order. Major ontological brain structure positioning is indicated with white lines and written above. Example statistically significant subdivisions are listed in red. FIG. 13G provide a bar graph of total statistically significant subdivisions versus no difference plotted for each major brain structure. Percent significant subregions is listed above each bar. FIG. 13H provides volumetric renderings of mean (top) SV40nls-EGFP or (middle) ArgiNLS-EGFP voxelized cell densities and the (bottom) statistically significant voxels (p<0.005) of the mean differences across all major brain structures. All bar graphs represent mean±SEM. The data for 13C was statistically analyzed using an unpaired Student's t-test. The data for 13D was statistically analyzed using a two-way ANOVA with Bonferroni post-hoc test for mean significant differences at each major region. The data for 13E was analyzed with linear regression. The data for 13F was analyzed with FDR-corrected multiple t-tests using the two-stage step-up method of Benjamini, Krieger, and Yekutieli. *p<0.05; **p<0.01. Acronyms: ISO=isocortex; OLF=olfactory areas; HPF=hippocampal formation; CTXsp=cortical subplate; CNU=cerebral nuclei; CBX=cerebellum; HY=hypothalamus; TH=thalamus; MB=midbrain; HB=hindbrain; AO=accessory olfactory area; Pir: piriform cortex; DLEnt: dorsolateral entorhinal cortex; SUB: subiculum; Tu=olfactory tubercle; MG=medial geniculate; MM=medial mammillary area; IC=inferior colliculus; KF=Koelliker-Fuse subnucleus.

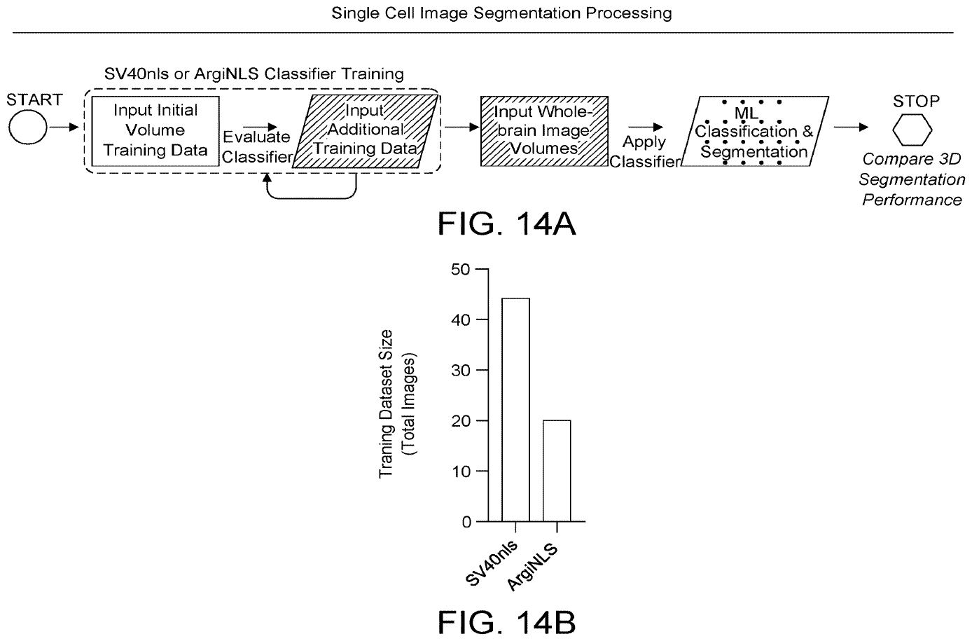

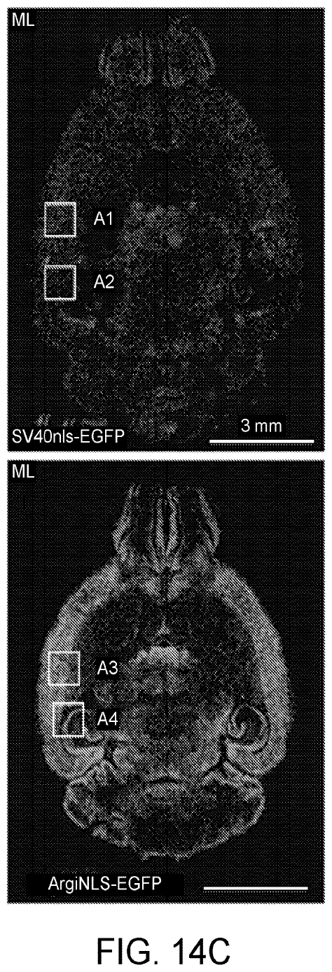

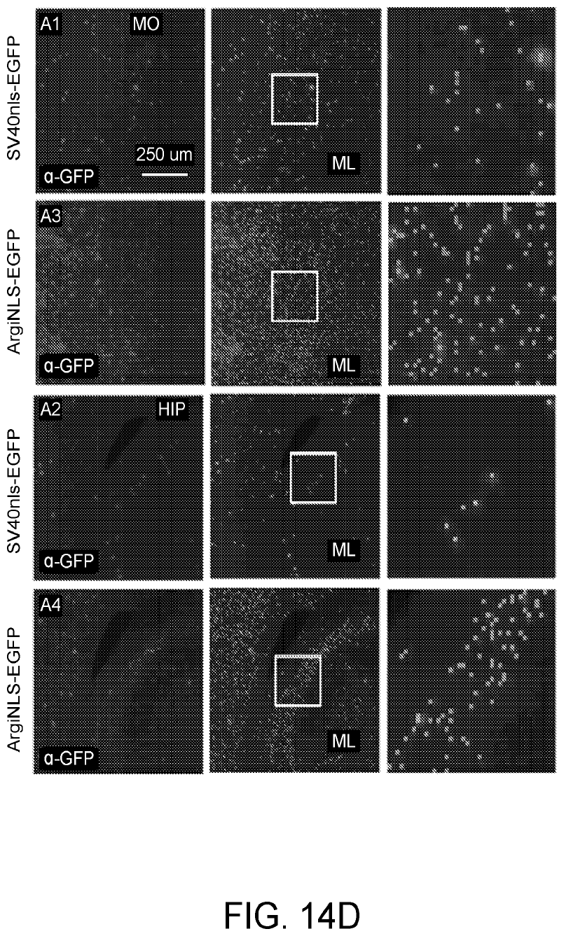

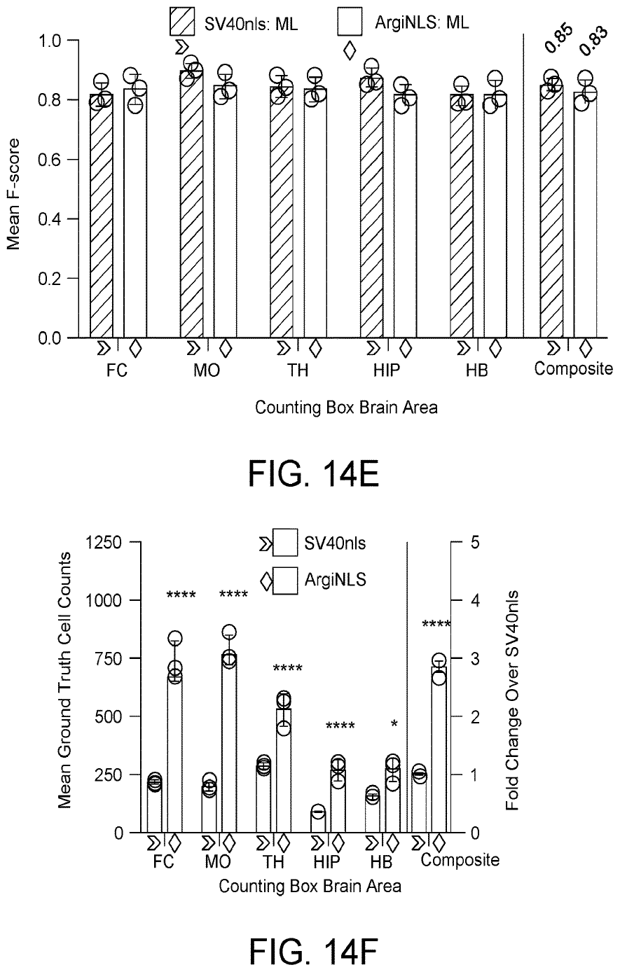

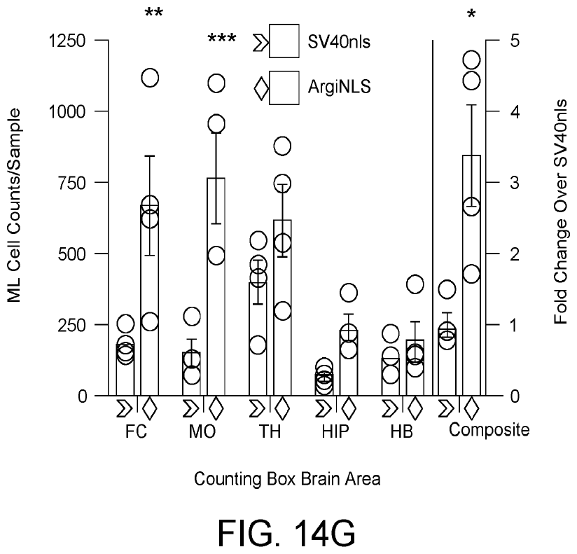

FIGS. 14A-14G illustrate validation of ML-based brain-wide segmentation of single-cells. FIG. 14A illustrates an example workflow of ML-based classifier training and application. FIG. 14B provides the training dataset size of classifiers used in whole-brain classification. FIG. 14C provides representative images of ML-based centroids of segmentation from FIG. 13B, with additional insets of crop squares highlighting example areas (MO and HIP) used for classifier metric calculations. FIG. 14D provides magnified images of cropped insets from 14C, showing raw image (left; pseudo-colored in red), raw image with overlaid ML classification (middle), and zoomed inset of overlaid image (right). FIG. 14E provides mean F-score performance for each classifier across counting box brain areas. Composite F-score (see methods) is plotted and listed to the right. FIG. 14F illustrates comparison of mean (n=3 samples) ground truth cell counts across counting box brain areas per expert. Composite fold change over SV40nls ground truth counts from all areas is plotted to the right. FIG. 14G illustrates a comparison of ML counts across counting box brain areas for each sample imaged (n=4). Composite fold change over SV40nls ML cell counts from all areas is plotted to the right. The data in 14E, 14F were statistically analyzed using a 2-way ANOVA with Bonferroni-corrected posthoc test. Composite values were statistically analyzed with an unpaired t test. p<0.01; **p<0.05; **p<0.01; ****p<0.001. Acronyms: FC=frontal cortex; MO=primary motor cortex; TH=thalamus; HIP=hippocampus; HB=hindbrain.

Enriched brain-wide segmentation of single-cells: It was next sought to determine ArgiNLS tag's impact on single-cell segmentation within a whole-brain mapping dataset using large-scale ML classification. PHP.eB SV40nls-EGFP and ArgiNLS-EGFP expressing viruses were systemically delivered, and iDISCO+whole-mount clearing and EGFP staining was performed to fluorescently label all the infected cells throughout the brain (FIG. 13A, top; FIG. 13B). Whole-brain samples were imaged with light sheet fluorescent microscopy (LSFM), and image datasets were run through a pipeline consisting of reference atlas registration, ML cellular segmentation, atlas segmentation of brain regions, and voxelized cell density analyses (FIG. 13A, bottom). Like the observations in 2D histology, SV40nls-EGFP staining produced higher image artifact and less cells counts than ArgiNLS-EGFP (FIG. 13B, left). Separate ML classifiers were therefore trained using ilastik software to achieve optimal single-cell classification performance, separately for each tag (FIG. 14A). ArgiNLS reduced the model training requirements in 3D, where SV40nls classifier training required a total dataset of 44 training images and ArgiNLS required 20 training images (FIG. 14B).

Following extensive classifier validation (SI Text), the whole-brain image processing pipeline was applied to create maps of systemic AAV.eB infection. It was found that ArgiNLS-EGFP significantly increases AAV.eB labeled whole-brain cell counts by 2.6-fold (SV40nls: 514,422±90,388; ArgiNLS: 1,362,466±268,889) (FIG. 13C). Additionally, it was determined if the whole-brain count difference was anatomically distributed preferentially or evenly across gross areas of the brain. Statistically significant increases of ArgiNLS-labeled cell densities were found specifically in the isocortex (ISO) (SV40nls: 1798±277, ArgiNLS: 5526±1156) and olfactory area (OLF) (SV40nls: 1162±211, ArgiNLS: 3753±609), suggesting these two brain areas are major drivers of the differences in whole-brain cell counts.

To identify subregional differences in labeled cell densities, 1216 anatomical annotations provided by the Unified atlas were used (Enhanced and unified anatomical labeling for a common mouse brain atlas|Nature Communications). First, it was determined if the general distribution of labeled cell patterning across subregions differ amongst genetic tags (FIG. 13E). Linear regression analysis revealed a statistically significant positive correlation of ArgiNLS versus SV40nls labeled cell densities of subregions, suggesting an equivalent anatomical distribution of labeled cells across subregions. However, with the exception of 30 subregions, 98% contained greater cell densities when labeled with ArgiNLS (FIGS. 13E, 13F).

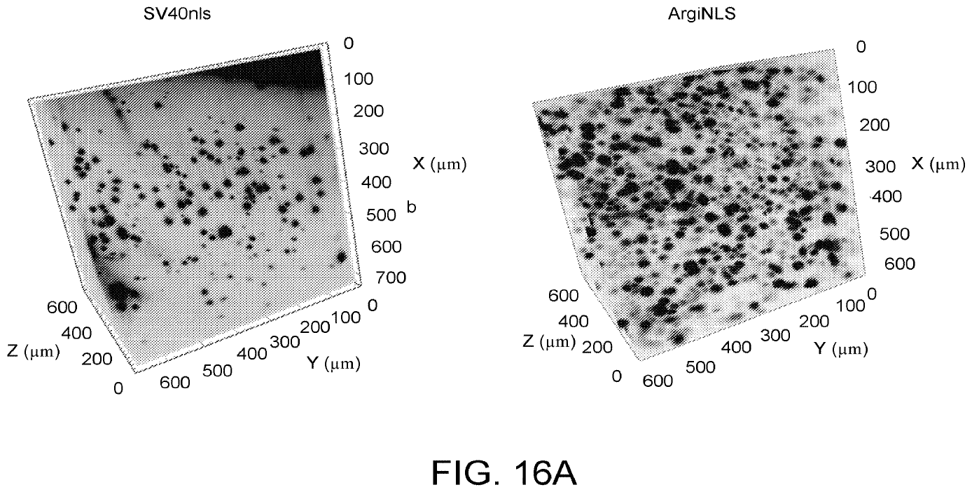

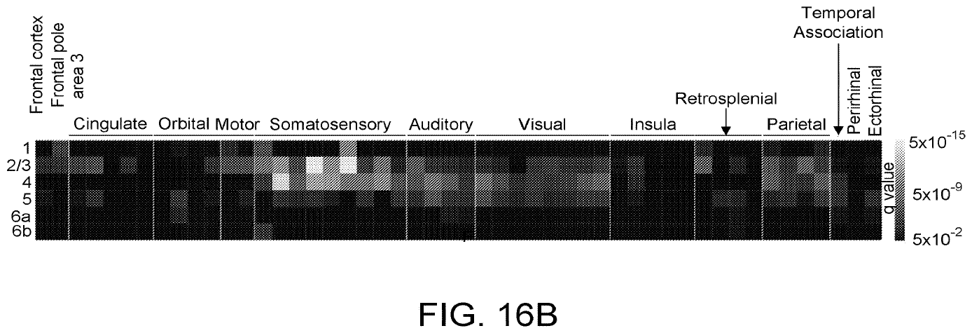

FIGS. 16A, 16B illustrate cortical differences in single-cell labeling and segmentation. FIG. 16A provides representative raw volumetric 3D image crops displaying EGFP-stained single-cell labeling content in a 600 um³ cube located in the barrel cortex. FIG. 16B provides a cortical heatmap of statistically significant differences from FIG. 13F, replotted across all cortical areas by layer.

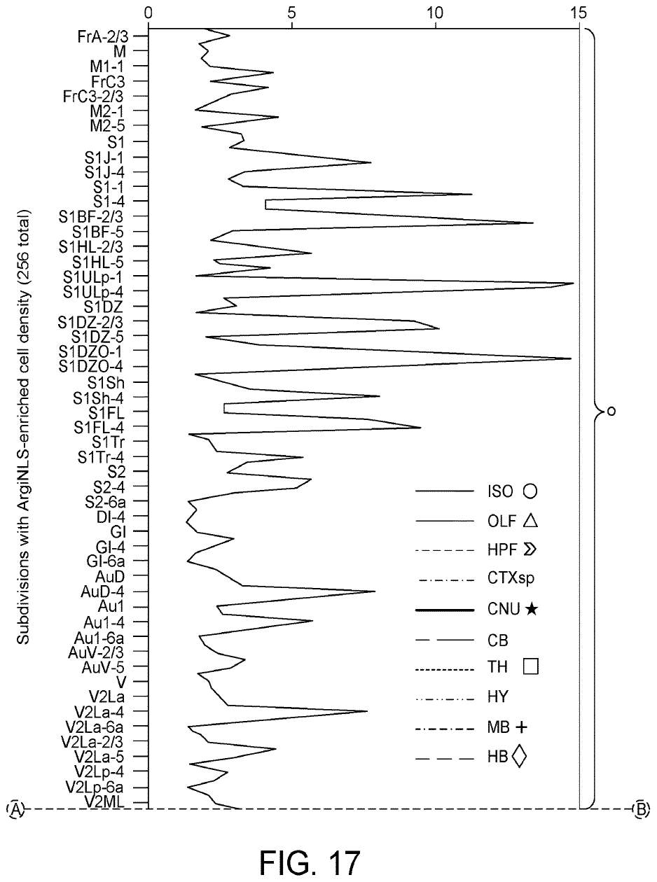

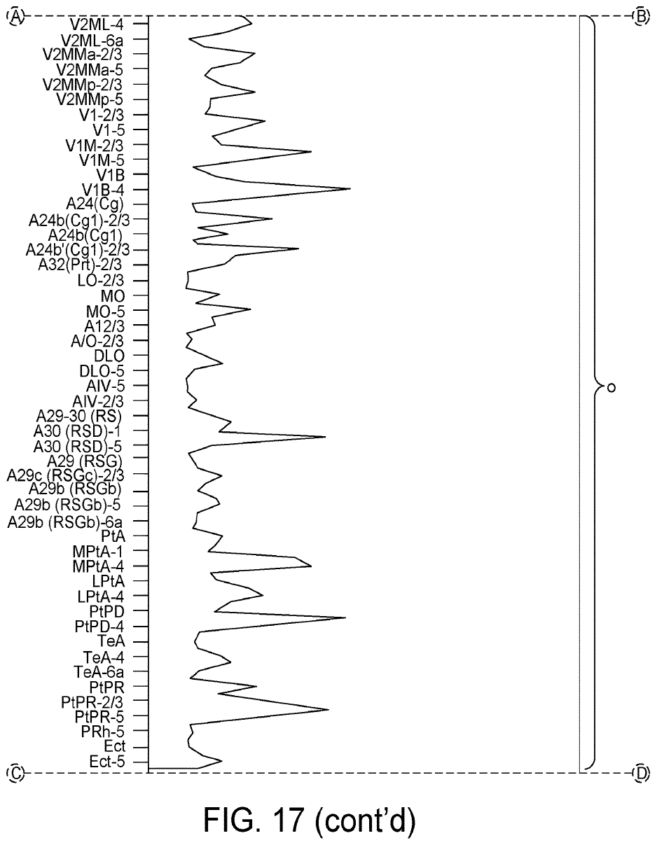

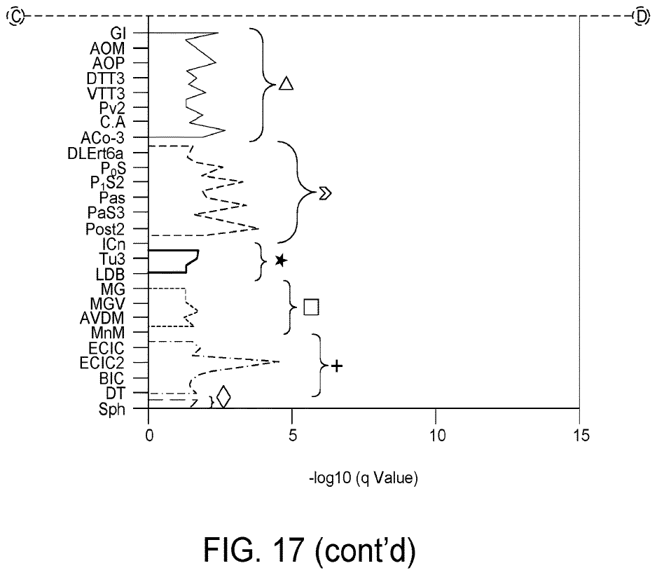

FIG. 17 illustrates ArgiNLS-enriched subregions. FIG. 17 provides an example filled line graph display of −log 10 q-values from all significant ArgiNLS-enriched subregions per major brain area. For legibility purposes, only every other subregion is listed. Full list and unabbreviated names are found in Table 3. From top to bottom, the y-axis reads: FrA-2/3, M, M1-1, FrC3, FrC3-2/3, M2-1, M2-5, S1, S1J-1, S1J-4, S1-1, S1-4, S1BF-2/3, S1BF-5, S1HL-2/3, S1HL-5, S1Ulp-1, S1Ulp-4, s1dz, s1dz-2/3, S1DZ-5, S1DZO-1, S1DZO-4, S1Sh, S1Sh-4, S1FL, S1FL-4, S1Tr, S1TTr-4, S2, S2-4, S2-6a, D1-4, GI, GI-4, GI-6a, AuD, AuD-4, Au1, Au1-4, Au1-6a, AuV-2/3, AuV-5, V, V2La, V2La-4, V2La-6a, V2Li-5, V2Lp-4, V2Lp-6a, V2ML, V2ML-4, V2ML-6a, V2Mma-2/3, V2Mma-5, V2MMp-2/3, V2MMp-5, V1-2/3, V1-5, V1M-2/3, V1M-5, V1B, V1B-4, A24 (Cg), A24b (Cg1)-2/3, A24b′ (Cg1), A24b′ (Cg1)-2/3, A32 (PrL)-2/3, LO-2/3, MO, MO-5, AI2/3, AID-2/3, DLO, DLO-5, AIP-5, AIV-2/3, A29-30 (RS), A30 (RSD)-1, A30 (RSD)-5, A29 (RSG), A29c(RSGc)-2/3, A29b (RSGb), A29b(RSGb)-5, A29b(RSGb)-6b, PtA, MptA-1, MptA-4, LptA, LptA-4, PtPD, PtPD-4, TeA, Tea-4, TeA-6a, PtPR, PtPR-2/3, PtPR-5, PRh-5, Ect, Ect-5, GI, AOM, AOP, DTT3, VTT3, Pir2, CxA, Aco-3, DLEnt6a, PsS, PsS2, PaS, PaS3, Post2, len, Tu3, LDB, MG, MGV, AVDM, MnM, ECIC, ECIC2, BIC, DT, and Sph.

Second, the identities of subregions significantly enriched in ArgiNLS-labeled cell density were screened for (FIG. 13F bottom; FIGS. 13G, 16A, 16B, 17). The most enriched gross regions—the ISO and OLF—contained a compilation of 60 (208/345; e.g., primary somatosensory area (S1)) and 23% (16/69; e.g., anterior olfactory area (AO), piriform cortex (Pir)) significant subregions, respectively (FIGS. 13G, 14A-14G). It was found that ISO cortical enrichment was predominant in layers 2/3 to 5, with heavy concentration in areas related to sensory processing (FIG. 17). Enrichment of ArgiNLS-labeled cell density was found in subregions broadly located in every major division of the brain (HPF: 13/82=16%, e.g., dorsolateral entorhinal cortex (DLEnt), subiculum (SUB); CTXsp: 1/23=4%, e.g., intermediate endopiriform claustrum (len); CNU: 4/120=3%, olfactory tubercle (Tu); CB: 1/65=1.5%, e.g., lateral cerebellar nucleus parvicellular part (LatPC); TH: 6/94=6%, e.g., medial geniculate nucleus (MG); HY: 1/104=1%, medial mammillary nucleus (MM); MB: 8/109=7%, e.g., inferior colliculus (IC); and HB: 2/198%=2%, e.g., Kolliker-fuse nucleus (KF)) (FIGS. 13G, 17). An orthogonal, voxel-by-voxel analysis of differences in brain-wide cell density was performed. Like the region-by-region analysis, significantly greater voxels were visualized across all major brain areas confirming wide-spread enrichment of labeled cell density by ArgiNLS (FIG. 13H).

ArgiNLS-optimized applications in volumetric brain cell counting: Spatially integrated features of brain structure and function can be studied through volumetric imaging paired with genetically-encoded fluorescent labels. For example, many transgenic lines are available for, among other purposes, guiding FP labeling amongst a constellation of cell-types of choice(64). Further, AAV technology is advancing rapidly as seen by recent serotype development directing FP labeling in retrograde, transsynaptic, or systemic fashions (D. L. Haggerty, et al., Molecular Therapy—Methods & Clinical Development 17, 69-82 (2020)). Since ArgiNLS optimizes single-cell FP labeling and segmentation, its utility for cell counting applications was established when incorporated into a variety of molecular genetic tools.

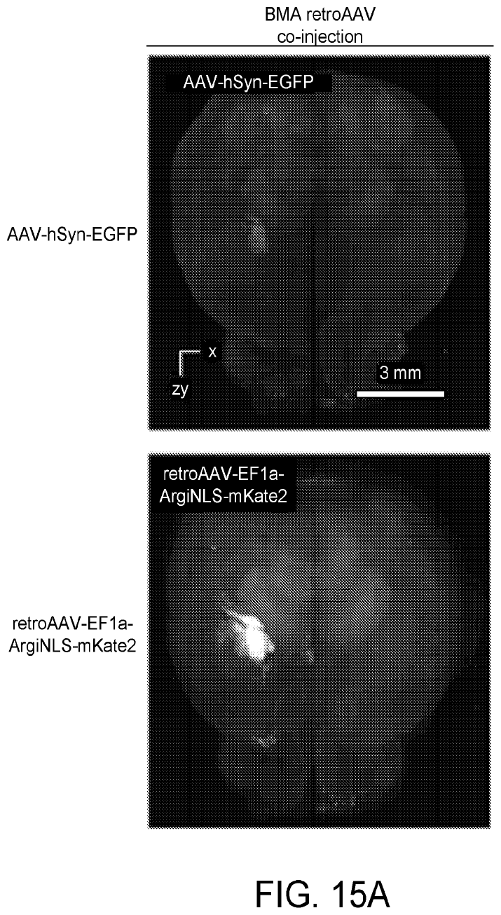

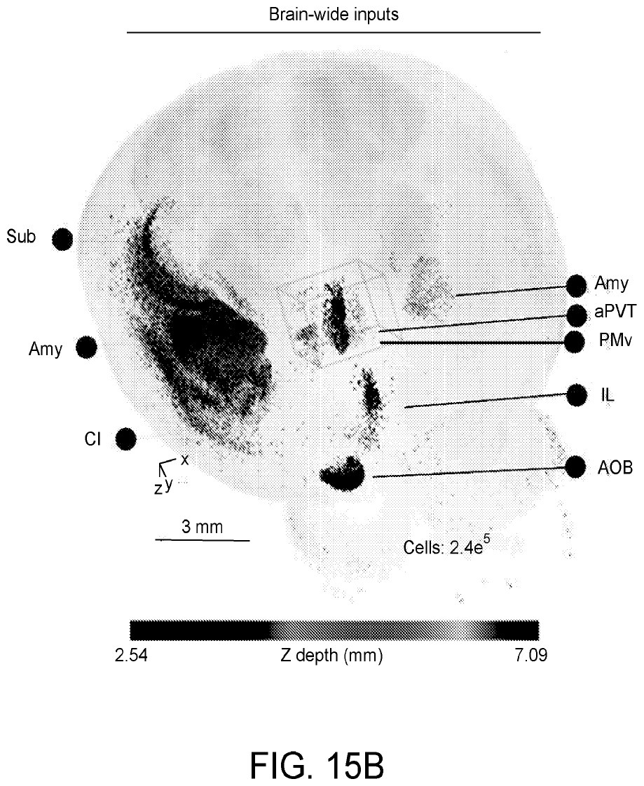

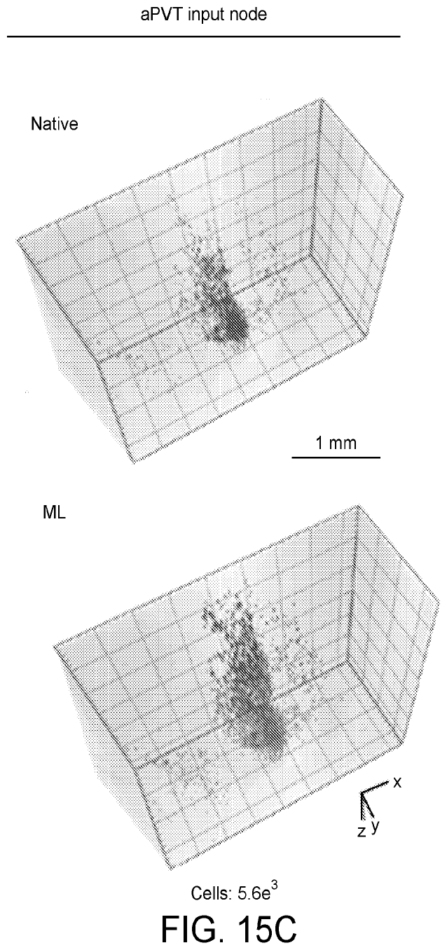

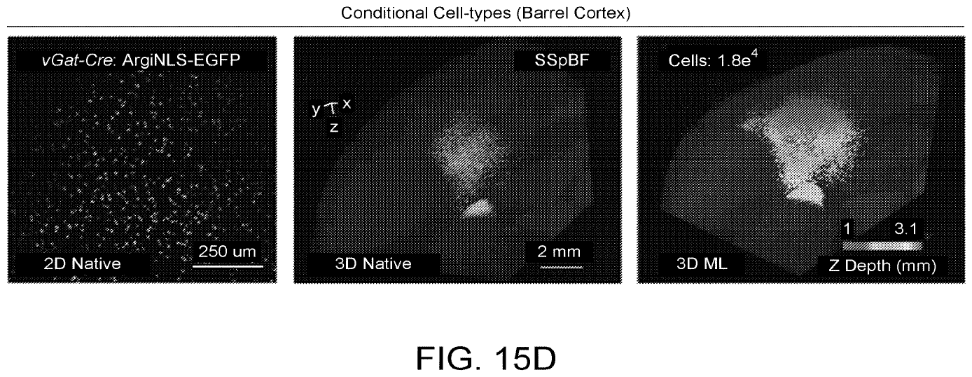

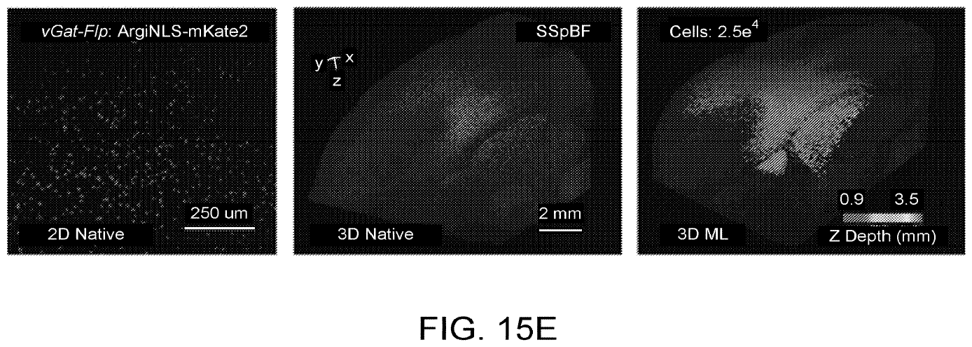

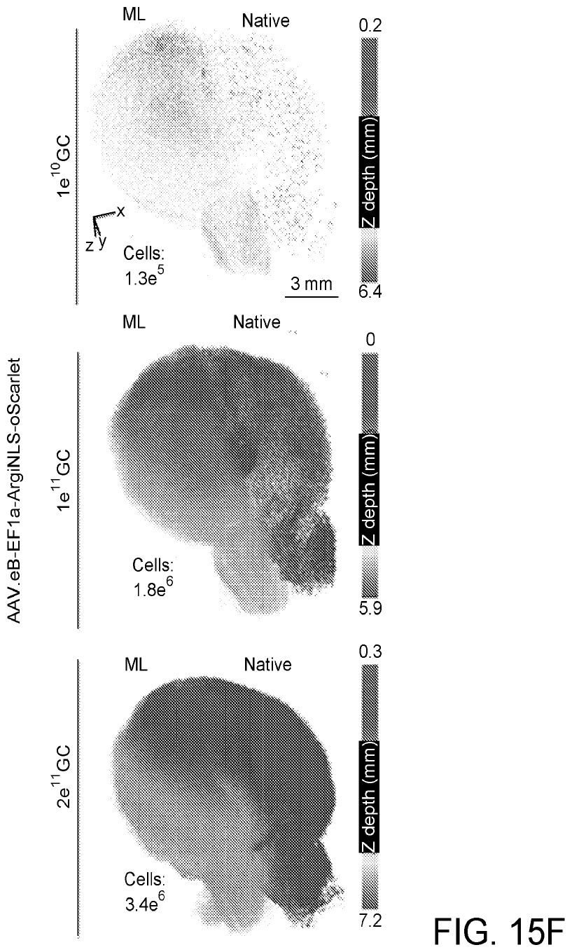

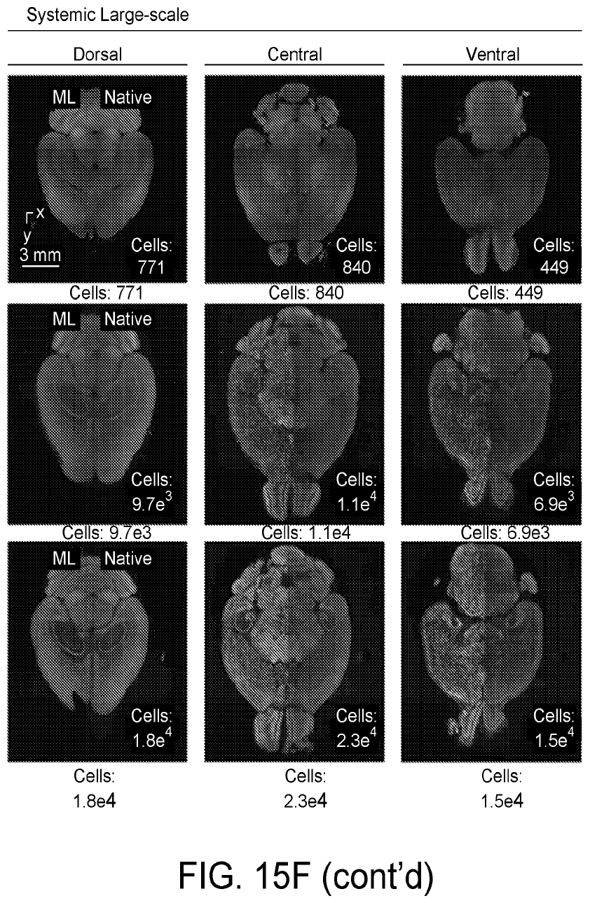

FIGS. 15A-15F illustrate optimized volumetric brain cell counting applications with ArgiNLS viral vectors. FIGS. 15A-15C illustrate example circuit mapping with ArgiNLS-mKate2-expressing retroAAV into the basomedia amygdala (BMA). In FIG. 15A, the top panel illustrates an example whole-brain volumetric rendering of AAV-hSyn-EGFP native expression labeling the injection site in green channel. In FIG. 15A, the bottom panel illustrates corresponding red channel image displaying native fluorescence of ArgiNLS-mKate2+ single-cell inputs. FIG. 15B illustrates a volumetric rendering of single-cell brain-wide input segmentations and their total counts using 3D ML, gradient color coded by Z depth. Total BMA input cells are listed in the bottom right. Main anatomical areas of local and long-range inputs are listed to the left and right, respectively. Sub=subiculum; Amy=amygdala; Cl=claustrum; aPVT=anterior paraventricular nucleus of the thalamus; PMv=ventral premammillary nucleus; IL=infralimbic cortex; AOB=accessory olfactory bulb FIG. 15C illustrates single-cells (native fluorescence: top; ML segmentation: bottom) and their total count from the anterior PVT (aPVT) input node cropped and expanded from the outlined inlay in 15B. FIGS. 15D, 15E provide conditional labeling of GABAergic single-cells in the barrel cortex using local (15D) Cre- or (15E) Flp-dependent AAV injections in vGAT-Cre or vGAT-Flp driver mice, respectively. In FIGS. 15D, 15E, the left panel provides 2D confocal image stacks displaying native conditional single-cell labeling from separate example brain samples. In FIGS. 15D, 15E, the middle panel provides example volumetric renderings of native conditional single-cell labeling from separate brain samples. In FIGS. 15D, 15E, the right panel Provides volumetric renderings of 3D ML-segmented single-cells and their total counts for each sample. FIG. 15F illustrates large-scale labeling of single-cells using systemic delivery of AAV.eB-EF1α-ArgiNLS-oScarlet virus at 1e¹⁰, 1e¹¹, or 2e¹¹ GC payloads. In FIG. 15F, the left panel illustrates example whole-brain volumetric renderings of combined hemispheric native ArgiNLS-oScarlet fluorescence (left hemisphere) and 3D ML segmentation (right hemisphere) of single-cells from separate example brain samples per viral condition. Total hemispheric cell counts are listed in the bottom left. In FIG. 15F, the right panel illustrates 2D horizontal planes of each sample across (top to bottom) dorsal, central, and ventral positions. Images display native ArgiNLS-oScarlet fluorescence with overlaid 3D ML classification on the right hemisphere and total cell counts per plane listed in the bottom right.

An AAV vector toolkit was compiled consisting of multiple ArgiNLS-tagged FPs, including bright green-(AausFP1(G. G. Lambert, et al., PLOS Biology 18 (2020)) and mGreenLantern (mGreenLantern. PNAS)), yellow-(mVenus ME(R. S. Cox, et al., Journal of Biological Engineering 4, 10 (2010))) and red-emitting (oScarlet(53)) variants within constitutive and conditional formats to offer experimental range. Several DREADD receptor-expressing constructs that co-express ArgiNLS-tagged FPs as a reporter gene were included, which enables high-fidelity labeling of DREADD-modulated single-cells with behavioral compatibility (Table 3) (B. L. Roth, Neuron 89, 683-694 (2016)). Aqueous-based SHIELD clearing (Y.-G. Park, et al., Nat Biotechnol 37, 73-83 (2019)) and axial swept LSFM imaging (K. M. Dean, et al., Nat Protoc 17, 2025-2053 (2022)) were applied to demonstrate ease of ArgiNLS-assisted segmentation of single-cells and their total count quantification using 3D ML from native fluorescence in 3D volumes. ArgiNLS-assisted quantification was demonstrated by generating and injecting a retrograde AAV-EF1α-ArgiNLS-mKate2 virus into the basomedial amygdala (BMA)—an amygdalar structure involved in anxiety and fear modulation (A. Adhikari, et al., Nature 527, 179-185 (2015))—to create an anatomical input map (FIGS. 15A-15C). Co-injected EGFP-expressing virus confirmed BMA injection placement, and native retro-ArgNLS-mKate2+ single-cells were readily identifiable at local and long-range distances from the injection site (FIG. 15A). 3D ML classification of brain-wide ArgiNLS-mKate2+cells quantified at total of 2.4e⁵ BMA inputs that were mainly distributed continuously throughout the ipsilateral cortical subplate including amongst multiple amygdala subregions (Amy) and claustrum (C), as well as within the subiculum of the hippocampus (FIG. 151B). Major long-range BMA input cells resided in 5 nodes including (from anterior to posterior) the accessory olfactory bulb (AOB), infralimbic cortex (IL), anterior paraventricular nucleus (aPVT) of the thalamus, contralateral amygdala, and ventral premammillary nucleus of the hypothalamus (PMv). The aPVT represented one of the largest long-range and singular thalamic node containing 5.6e³ input cells, highlighting the existence of a putative aPVT-BMA circuit motif of unknown behavioral function.

TABLE 3
ArgiNLS-tagged viral vector toolkit for optimized cell counting.

| Construct | Promoter | FP Gene | Ex/Em | Condition | Addgene ID |
|---|---|---|---|---|---|
| pAAV-EF1α-ArgiNLS-EGFP | EF1α | EGFP | 488/507 | Constitutive | 220594 |
| pAAV-EF1α-ArgiNLS-AausFP1 | EF1α | AausFP1 | 504/510 | Constitutive | 220595 |
| pAAV-EF1α-ArgiNLS-mGreenLantern | EF1α | mGreenLantern | 503/514 | Constitutive | 220596 |
| pAAV-EF1α-ArgiNLS-mVenus-Q69M (ME) | EF1α | mVenus ME | 515/528 | Constitutive | 220597 |
| pAAV-EF1α-ArgiNLS-oScarlet | EF1α | oScarlet | 569/594 | Constitutive | 220598 |
| pAAV-EF1α-ArgiNLS-mKate2 | EF1α | mKate2 | 588/633 | Constitutive | 220599 |
| pAAV-EF1α-ArgiNLS-miRFP670 | EF1α | miRFP670 | 642/670 | Constitutive | 220600 |
| pAAV-CAG-FLEX-ArgiNLS-EGFP | CAG | EGFP | 488/507 | Cre-dependent | 220601 |
| pAAV-CAG-FLEX-ArgiNLS-AausFP1 | CAG | AausFP1 | 504/510 | Cre-dependent | 220602 |
| pAAV-CAG-FLEX-ArgiNLS-oScarlet | CAG | oScarlet | 569/594 | Cre-dependent | 220603 |
| pAAV-CAG-FLEX-ArgiNLS-mKate2 | CAG | mKate2 | 588/633 | Cre-dependent | 220604 |
| pAAV-CAG-fDIO-ArgiNLS-AausFP1 | CAG | AausFP1 | 504/510 | Flp-dependent | 220605 |
| pAAV-CAG-fDIO-ArgiNLS-oScarlet | CAG | oScarlet | 569/594 | Flp-dependent | 220606 |
| pAAV-CAG-fDIO-ArgiNLS-mKate2 | CAG | mKate2 | 588/633 | Flp-dependent | 220607 |
| pAAV-EF1α-DIO-3x HA-hM3D(Gq)- | EF1α | oScarlet | 569/594 | Cre-dependent/hM3D(Gq) | 220612 |
| P2A-ArgiNLS-oScarlet | | | | DREADD co-expressing | |
| pAAV-EF1α-DIO-FLAG-hM4D(Gi)- | EF1α | AausFP1 | 504/510 | Cre-dependent/hM4D(Gi) | 220613 |
| P2A-ArgiNLS-AausFP1 | | | | DREADD co-expressing | |
| pAAV-EF1α-DIO-FLAG-KORD-P2A- | EF1α | oScarlet | 569/594 | Cre-dependent/KORD | 220614 |
| ArgiNLS-oScarlet | | | | DREADD co-expressing | |
| pAAV-hSyn-DIO-3x HA-hM3D(Gq)- | hSyn | oScarlet | 569/594 | Neuron-specific/Cre- | 220608 |
| P2A-ArgiNLS-oScarlet | | | | dependent/hM3D(Gq) | |
| | | | | DREADD co-expressing | |
| pAAV-hSyn-DIO-FLAG-hM4D(Gi)- | hSyn | AausFP1 | 504/510 | Neuron-specific/Cre- | 220609 |
| P2A-ArgiNLS-AausFP1 | | | | dependent/hM3D(Gq) | |
| | | | | DREADD co-expressing | |
| pAAV-hSyn-DIO-FLAG-KORD-P2A- | hSyn | oScarlet | 569/594 | Neuron-specific/Cre- | 220610 |
| ArgiNLS-oScarlet | | | | dependent/hM3D(Gq) | |
| | | | | DREADD co-expressing | |
| pAAV-hSyn-DIO-FLAG-KORD-P2A- | hSyn | n/a | n/a | Neuron-specific/Cre- | 220611 |
| 3x HA-hM3D(Gq) | | | | dependent/KORD and | |
| | | | | hM3D(Gq) DREADD | |
| | | | | co-expressing | |

Table 3 provides details about the NLS-tagged vectors described in this Example.

Second, AAV.eB-CAG-FLEX-ArgiNLS-EGFP and AAV.eB-CAG-fDIO-ArgiNLS-mKate2 virus were generated and locally injected into the barrel cortex of vGat-Cre or vGat-Flp driver mice, respectively, to conditionally label and 3D segment GABAergic single-cells (FIGS. 15D, 15E). High SNR-labeled single-cells were identically observed in Cre- or Flp-dependent conditions in 2D (FIGS. 15D, 15E, left). In 3D, a clear distribution of single-cell-labeled viral spread was observed and devoid of extranuclear signal artifact in both Ore- and Flp-dependent conditions (FIGS. 15D, 15E, middle), with 3D ML segmenting 1.8e⁴ and 2.5e⁴ GABAergic cells, respectively (FIGS. 15D, 15E, right).

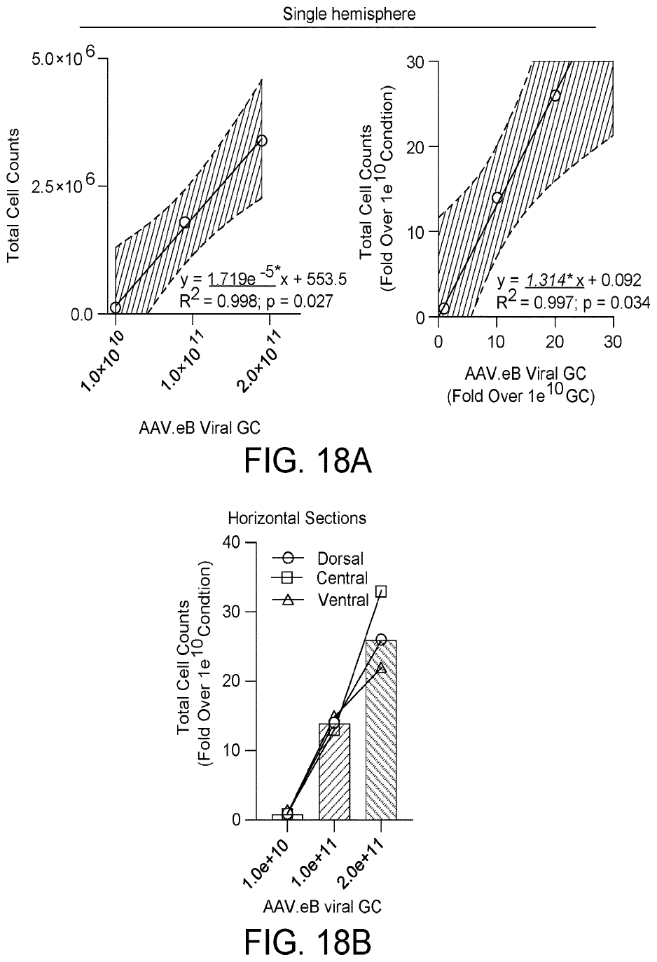

FIGS. 18A, 18B illustrate volumetric quantification of single-cells from systemic PHP.eB ArgiNLS-oScarlet labeling with 3 genetic payload amounts. In FIG. 18A, the left panel illustrates linear regression of total hemisphere cell counts versus AAV.eB viral GC payload amount. In FIG. 18A, the right panel illustrates linear regression of fold-expressed cell counts versus fold-expressed GC payload. FIG. 18B provides a bar graph of total cell counts from horizontal sections corresponding to 3 anatomical locations, taken from whole-brain samples.

Finally, an AAV.eB-EF1α-ArgiNLS-oScarlet virus was generated and systemically delivered to demonstrate robust native fluorescence labeling of single-cells and their ease of segmentation of large cell amounts ubiquitously throughout the brain (FIG. 15F). To gauge sensitivity of detection levels enabled by ArgiNLS, 3 amounts—1e¹⁰ (1×), 1e¹¹ (10×), and 2e¹¹ (20×)—of viral GC payloads were systemically delivered. 3D renderings of native signal displayed sharply contrasted single-cells distributed widely throughout the brain, and scaled with viral GC amount (FIG. 15F, left). 3D ML quantification of single hemispheres segmented 1.3e⁵, 1.8e⁶ (14-fold), and 3.4e⁶ (26-fold) single-cells for 1e¹⁰, 1e¹¹, and 2e¹¹ GC payloads, respectively, or 1 cell labeled for every 58,173 viral GCs delivered (FIG. 15F, left; FIG. 18A, left). Expressing fold change values over the lowest 1e¹⁰ GC values indicated the utility of ArgiNLS-oScarlet to precisely label and segment 1.3-fold cell count increases for every 1-fold increase in viral GC delivered, within the 1 to 20-fold (i.e., 1e¹⁰-2e¹¹) GC range tested (FIG. 18A, right). This total single-cell detection by viral GC relationship was maintained evenly throughout the brain as observed in cell counts across separate horizontal planes located in dorsal, central, and ventral anatomical positions (FIG. 15F, right; FIG. 18B).

### Discussion

Volumetric imaging methods can be used to unveil spatially integrated representations—or maps—of brain structure and function at the direct level of single-cells. Pivotal to this approach is the ability of genetically-encoded fluorescent labels to discriminate single-cells within dense and complex brain tissue. In this Example, a new nuclear-localizing genetic tag strategy named ArgiNLS was developed and characterized that optimizes FP labeling of single-cells, without off-target effects on cellular physiology or animal behavior. By extension, ArgiNLS provides seamless compatibility and enrichment of ML-automated brain cell segmentation across small-to-large scale image datasets of various applications, demonstrating its use as an improved single-cell resolution brain mapping genetic tool.

Unlike other recently developed synthetic NLS approaches created for enhanced genome-editing(K. Luk, et al., GEN Biotechnology 1, 271-284 (2022); T. Thumberger, et al., eLife 11 (2022); P. Hu, et al, G3 (Bethesda) 8, 823-831 (2018)), the ArgiNLS approach for image segmentation is based on a well-understood molecular foundation. This approach uses a poly-R-based rerouting mechanism to reduce extra-nuclear NLS-tagged FP localization by attracting it into the nucleolus. Optimal tag efficacy of ArgiNLS occurs when the BP cNLS sequence is combined with a specific poly-R length (R7) that titrates FP localization into a balanced nucleoplasmic/nucleolar composition with optimal native brightness. MP cNLS combinations fail to reduce extra-nuclear artifact in combinations up to 11 Rs, indicating that the function of R7 synergism on ectopically-fused NLS activity requires sequence and/or length characteristics of a BP NLS. The function of this configuration within cells outside of the CNS and amongst varied species was not determined in this Example, and therefore context-specific modification may be required. However, as demonstrated in mouse brain here, the ArgiNLS strategy lays a robust framework for tailoring function through the parameterization of BP NLS sequence selection and/or poly-R lengths.

The nature of the poly-R7 component of ArgiNLS additionally provides functional fidelity across a wide variety of FPs, that can provide spectral flexibility for single-cell labeling, and potential nuclear localization of other non-FP proteins, through its dual role as a protein linker. This capability was demonstrated on an initial group of spectrally separate FPs. However, further validation amongst a larger FP selection and with non-FP proteins for applications outside of image segmentation is necessary to strengthen the generalization of this linker-like modularity.

NLS performance of ArgiNLS in vivo demonstrates several advantages over current SV40nls tag or H2B fusion single-cell labeling approaches. First, ArgiNLS causes FP nuclear restriction that is equally robust whether an AAV is delivered locally or systemically to the brain. ArgiNLS eliminates background fluorescence-producing, extra-nuclear signal that is consistently observed with SV40nls. ArgiNLS elicits consistent nuclear-restricted FP labelling across major cortical cell classes. This property enables improved ML image classification of intact whole brain tissue, which is characterized by a reduction in model training requirements and coupled to increased precision and recall of segmentations. Unlike H2B fusion35, these benefits were not associated with deleterious effects to neural physiology or animal behavior. Therefore, ArgiNLS is appropriate for genetic labeling experiments of single-cells that require concomitant behavioral procedures paired with AAV infection.

The in vivo use of ArgiNLS highlighted how the selection of genetic labelling strategy can significantly influence whole-brain single-cell segmentations, with an effective doubling of cell counts across subregions located in all major brain structures. Additionally, since a fraction of wild-type FP molecules misfold or do not form a proper chromophore (O. V. Stepanenko, et al., International Review of Cell and Molecular Biology, (2013), pp. 221-278; B. P. Cormack, et al., Gene 173, 33-38 (1996)), poly-R driven interaction with nucleolar ribosomal RNA (R. M. Martin, et al., Nucleus 6, 314-325 (2015)) and/or nucleolus-dependent changes of pH appear to provide a stabilizing influence in aggregation or folding of ArgiNLS-tagged FPs, leading to more single-cells detected.

Finally, ArgiNLS can be applied in various volumetric cell labeling applications to resolve cell counts seamlessly and rapidly with 3D ML segmentation. The described experiments begin to demonstrate ArgiNLS compatibility with, and enhancement of, anatomical input mapping, conditional segmentation of cell-types, and mapping a wide range of titrated cell counts within 3D brain volumes using various AAV vectors. However, ArgiNLS may also be incorporated into other molecular genetic tools that will provide improvements to single-cell counting applications including intersectional mapping (O. V. Stepanenko, et al., International Review of Cell and Molecular Biology, (2013), pp. 221-278) with or without multiplexing of spectrally separate ArgiNLS-tagged FPs, transsynaptic anatomical tracing (D. L. Haggerty, et al., Molecular Therapy—Methods & Clinical Development 17, 69-82 (2020)), activity-dependent reporting (L. DeNardo, L. Luo, Current Opinion in Neurobiology 45, 121-129 (2017)), and transgenic reporter mouse lines for genomically-encoded expression (S. Navabpour, et al., Neurosci Biobehav Rev 108, 732-748 (2020)). The creation of future ArgiNLS-tagged expression vectors for gene delivery from other commonly used viral vectors (S. Ghosh, et al., Applied Biosafety 25, 7-18 (2020)), as well as gene-targeting vectors for the creation of reporter mouse lines, will help expand optimized single-cell labeling and segmentation amongst broad genetic delivery options.

In conclusion, the simple yet powerful ArgiNLS genetic tag strategy described here advances streamlined quantification of single-cells from small-to-large scale brain image datasets with enriched labeling capacity and enhanced ML capabilities.

### EXAMPLE CLAUSES

1. A method of delivering a peptide or protein to a cell nucleus, the method including:
contacting a eukaryotic cell with the peptide or protein including a nuclear targeting tag including:
   a bipartite nuclear localization sequence (NLS); and
   a linker including four, five, six, seven, eight, nine, or ten arginines.
2. A method of delivering an agent to a nucleus of a cell, the method including:
   providing, to a sample or a subject including the cell, the agent conjugated to a nuclear targeting tag, the nuclear targeting tag including:
      an NLS; and
      a polyarginine linker including three, four, five, six, seven, eight, nine, ten, eleven, or twelve arginines.
3. The method of clause 1 or 2, wherein the agent includes at least one of a peptide, a protein, a detectable label, a therapeutic agent, a vector, nucleic acid, or a chemical agent.
4. The method of clause 3, wherein the therapeutic agent includes at least one of a gene therapy agent, a small molecule, a peptide, a protein, an immune therapy, a vaccine, or an enzyme.
5. The method of clause 4, wherein the gene therapy agent includes at least one of a microRNA, an antisense oligonucleotide, a short interfering RNA, a short hairpin RNA, a plasmid, a viral vector, a non-viral vector, a clustered regularly interspaced short palindromic repeats (CRISPR) RNA, or a Cas protein.
6. The method of any of clauses 3-5, wherein the detectable label includes at least one of a fluorescent label, a luminescent label, a radioisotope, a contrast reagent, or a quantum dot.
7. The method of clause 6, wherein the fluorescent label includes at least one of enhanced green fluorescent protein (EGFP), oScarlet, mKate2, miRFP670, AausFP1, mVenus-ME, E2-Crimson or mGreenLantern.
8. The method of any of clauses 1-7, wherein the cell is a brain cell, a central nervous system cell, a heart cell, an epithelial cell, an endothelial cell, a muscle cell, an immune cell, an adipocyte, an osteocyte, or a stem cell.
9. The method of any of clauses 1-8, wherein the cell is a glutamatergic cell or a gamma-aminobutyric acid(GABA)ergic cell.
10. The method of any of clauses 1-9, wherein the cell is a mammalian cell.
11. The method of clause 10, wherein the mammalian cell is mouse cell, a rat cell, a non-human primate cell, or a human cell.
12. The method of any of clauses 1-11, wherein the providing includes applying a fluid including the agent conjugated to the nuclear targeting tag.
13. The method of clause 12, wherein the pipetting is to a brain slice or a tissue sample.
14. The method of any of clauses 1-13, wherein the providing includes administering the agent conjugated to the nuclear targeting tag to a living subject.
15. The method of clause 14, wherein the living subject is a mammal.
16. The method of clause 14 or 15, wherein the living subject is a mouse, a rat, a non-human primate, or a human.
17. The method of any of clauses 14-16, wherein the administering to a living subject is through injection.
18. The method of clause 17, wherein the injection includes intravenous injection, intraparenchymal injection into brain tissue, intracerebroventricular (ICV) injection, intra-cisterna magna (ICM) injection, or intrathecal injection.
19. The method of any of clauses 14-18, wherein the living subject is a subject in need thereof,
   wherein the agent includes a therapeutic agent, and
   wherein the administering includes administering a therapeutically effective amount of the therapeutic agent to the living subject, thereby treating the living subject.
20. The method of any of clauses 1-19, wherein the agent includes or is conjugated to a detectable label, the method further including:
   detecting, in the sample, the detectable label.
21. The method of clause 20, wherein the detecting includes generating an image depicting the sample or performing fluorescence activated cell sorting.
22. The method of clause 21, further including:
   generating a segmentation mask by inputting, into a machine learning (ML) model trained to segment cells, the image depicting the sample.
23. The method of any of clauses 20-22, wherein the agent is a first agent, and the detectable label is a first detectable label, the method further including:
   providing, to the subject, a second agent including or conjugated to a second detectable label;
   detecting, in the sample, the second detectable label; and
   comparing a level or a distribution of the first detectable label to a level or a distribution of the second detectable label.
24. The method of any of clauses 1-23, wherein the NLS is a bipartite NLS including the sequence set forth in SEQ ID NO: 2, SEQ ID NO: 4, SEQ ID NO: 6.
25. The method of any of clauses 1-24, wherein the nuclear targeting tag includes the sequence set forth in SEQ ID NO: 1, SEQ ID NO: 3, or SEQ ID NO: 5.
26. The method of any of clauses 1-25, wherein the NLS is a bipartite NLS including the sequence (K/R)-X₁₀₋₁₂-(K/R/X), where K/R represents any combination of any length of K and R, X represents any amino acid, and K/R/X represents any combination of any length of K, R, and X.
27. The method of any of clauses 3-26, wherein the nuclear targeting tag is at or near a N terminus or a C terminus of the peptide or protein.
28. The method of any of clauses 1-27, wherein the providing includes transforming the cell with an expression construct including a nucleic acid sequence encoding the agent.
29. The method of any of clauses 1-28, wherein the polyarginine linker includes at least seven arginines.
30. An isolated protein or peptide including a nuclear targeting tag including:
   an NLS; and
   a linker including three, four, five, six, seven, eight, nine, ten, eleven, or twelve arginines.
31. An isolated agent including a nuclear targeting tag including:
   an NLS; and
   an arginine linker including three, four, five, six, seven, eight, nine, ten, eleven, or twelve arginines.
32. The isolated agent of clause 30 or 31, wherein the isolated agent further includes at least one of a protein, a peptide, a detectable label, a therapeutic agent, a vector, a nucleic acid, or a chemical agent.
33. The isolated agent of clause 32, wherein the detectable label includes at least one of a fluorescent label, a luminescent label, a radioisotope, a contrast reagent, or a quantum dot.
34. The isolated agent of clause 33, wherein the fluorescent label includes at least one of EGFP, oScarlet, mKate2, miRFP670, AausFP1, mVenus-ME, or mGreenLantern.
35. The isolated agent of any of clauses 30-34, wherein the NLS is a bipartite NLS including the sequence set forth in SEQ ID NO: 2, SEQ ID NO: 4, or SEQ ID NO: 6.
36. The isolated agent of any of clauses 30-35, wherein the nuclear targeting tag includes the sequence set forth in SEQ ID NO: 1, SEQ ID NO: 3, or SEQ ID NO: 5.
37. The isolated agent of any of clauses 30-36, wherein the NLS is a bipartite NLS including the sequence (K/R)-X₁₀₋₁₂-(K/R/X), where K/R represents any combination of any length of K and R, X represents any amino acid, and K/R/X represents any combination of any length of K, R, and X.
38. A host cell including the isolated protein or peptide of clause 30 or the isolated agent of any one of clauses 31-37.
39. The host cell of clause 38, wherein the host cell expresses the isolated agent.
40. The host cell of clause 38 or 39, wherein the isolated agent is targeted to the nucleolus of the host cell.
41. The host cell of any of clauses 38-40, wherein the host cell is a mammalian cell.
42. The host cell of any of clauses 38-41, wherein the host cell is a mouse cell, a rat cell, a non-human primate cell, or a human cell.
43. A host animal including the isolated protein or peptide of clause 30, the isolated agent of any one of clauses 31-37, or the host cell of any one of clauses 38-42.
44. The host animal of clause 43, wherein the isolated agent is expressed over an extended period of time.
45. The host animal of clause 44, wherein the extended period of time is greater than 3 weeks.
46. The host animal of any of clauses 43-45, wherein the host animal is a mammal.
47. The host animal of any of clauses 43-46, wherein the host animal is a mouse, a rat, a non-human primate, or a human.
48. An imaging system, including:
   the isolated protein or peptide of clause 30, the isolated agent of any of clauses 31-37, or the host cell of any of clauses 38-42, wherein the nuclear targeting tag is conjugated to a detectable label;
   a sensor configured to detect the detectable label; and
   a processor configured to determine a level or a distribution of the detectable label.
49. The imaging system of clause 48, which is a system for in vivo or in vitro brain imaging.
50. The imaging system of clause 48 or 49, which is a system for single-cell imaging.
51. The imaging system of any of clauses 48-50, wherein the sensor includes a charge-coupled device (CCD) camera or a complementary metal-oxide-semiconductor (CMOS) camera.
52. The imaging system of any of clauses 48-51, wherein the processor is configured to apply a machine learning algorithm for cellular segmentation.
53. An expression construct, including:
   a nucleic acid encoding the isolated protein or peptide of clause 30 or the isolated agent of any of clauses 31-37, the nucleic acid being functionally linked to a promoter; or
   a nucleic acid encoding:
      a nuclear targeting tag including:
         an NLS; and
         an arginine linker including three, four, five, six, seven, eight, nine, ten, eleven, or twelve arginines;
   the nucleic acid being adjacent to a multicloning site.
54. The expression construct of clause 53, wherein the promoter includes any naturally occurring or synthetic promoter used for gene-overexpression, tissue-specific expression, or inducible expression.
55. The expression construct of clause 53 or 54, wherein the promoter includes an EF1a promoter, a human synapsin 1 (hSyn1) promoter, a tetracycline-responsive element (TRE) promoter, a Fos promoter, an enhanced synaptic activity-responsive element (E-SARE) promoter, a cytomegalovirus (CMV) promoter, a CMV immediate enhancer/β-actin (CAG) promoter, a beta-globin promoter, a rhodopsin (Rho) promoter, SV40 early promoter, a Hsp68 minimal promoter (proHSP68), or a Rous Sarcoma Virus (RSV) long-terminal repeat (LTR) promoter.
56. The expression construct of any of clauses 53-55, wherein the NLS is a bipartite NLS including the sequence set forth in SEQ ID NO: 2, SEQ ID NO: 4, SEQ ID NO: 56, or (K/R)-X₁₀₋₁₂-(K/R/X), where K/R represents any combination of any length of K and R, X represents any amino acid, and K/R/X represents any combination of any length of K, R, and X.
57. The expression construct of clause 53, wherein the nuclear targeting tag includes the sequence set forth in SEQ ID NO: 1, SEQ ID NO: 3, or SEQ ID NO: 5.
58. A vector including the expression construct of any of clauses 53-57.
59. The vector of clause 58, wherein the vector includes a viral vector, an adenovirus vector, an adeno-associated virus vector, a lentiviral vector, a herpes simplex virus vector, an over-expression vector, a cloning vector, or a gene-modifying vector.
60. A method of cell imaging including:
   expressing, in a cell, a peptide or protein including:
      a detectable label; and
      a nuclear targeting tag including:
         an NLS; and
         an arginine linker including at least seven arginines; and obtaining an image of the cell.
61. A method including:
   expressing, in a cell, a detectable label conjugated to a nuclear targeting tag, the nuclear targeting tag including:
      an NLS; and
      an arginine linker including three, four, fix, six, seven, eight, nine, ten, eleven, or twelve arginines; and
   generating an image depicting the cell.
62. The method of clause 61, wherein the cell is a brain cell, a central nervous system cell, a heart cell, an epithelial cell, an endothelial cell, a muscle cell, an immune cell, an adipocyte, an osteocyte, or a stem cell.
63. The method of clause 61 or 62, wherein the cell is a glutamatergic cell or a GABAergic cell.
64. The method of any of clauses 61-63, wherein the cell is a mouse cell, a rat cell, or a human cell.
65. The method of any of clauses 61-64, wherein the detectable label includes at least one or a fluorescent label, a luminescent label, a radioisotope, a contrast reagent, or a quantum dot.
66. The method of any of clauses 61-65, wherein the detectable label is further conjugated to at least one of a peptide, a protein, a detectable label, a gene therapy agent, a therapeutic agent, a vector, a nucleic acid, or a chemical agent.
67. The method of clause 61, wherein the NLS is a bipartite NLS including the sequence set forth in SEQ ID NO: 2, SEQ ID NO: 4, SEQ ID NO: 6, or (K/R)-X₁₀₋₁₂-(K/R/X), where K/R represents any combination of any length of K and R, X represents any amino acid, and K/R/X represents any combination of any length of K, R, and X.
68. The method of clause 61, wherein the nuclear targeting tag includes the sequence set forth in SEQ ID NO: 1, SEQ ID NO: 3, or SEQ ID NO: 5.
69. A kit, including:
   a nuclear targeting tag or a nucleic acid encoding the nuclear targeting tag, the nuclear targeting tag including:
      an NLS; and
      an arginine linker including three, four, five, six, seven, eight, nine, ten, eleven, or twelve arginines.
70. The kit of clause 69, wherein the nuclear targeting tag is conjugated to a detectable label.
71. The kit of clause 69 or 70, further including:
   reagents to conjugate the nuclear targeting tag to an agent.
72. The kit of clause 71, wherein the agent includes at least one of a peptide, a protein, a detectable label, a gene therapy agent, a therapeutic agent, a vector, a nucleic acid, or a chemical agent.
73. A method of studying health of the host cell of any of clauses 38-42 or the host animal of any of clauses 43-47, including:
   evaluating a parameter representing a normal function of the host cell or the host animal; and
   determining that the parameter is within a normal physiological range for the host cell or the host animal.
74. The method of clause 73, wherein evaluating the parameter includes taking more than one measurement of a characteristic of the host cell or the host animal over a period of time selected from: at least one week, two or more weeks, three or more weeks, at least a month, or more than a month.

### Closing Paragraphs:

The features disclosed in the foregoing description, or the following claims, or the accompanying drawings, expressed in their specific forms or in terms of a means for performing the disclosed function, or a method or process for attaining the disclosed result, as appropriate, may, separately, or in any combination of such features, be used for realizing implementations of the disclosure in diverse forms thereof.

As will be understood by one of ordinary skill in the art, each implementation disclosed herein can comprise, consist essentially of or consist of its particular stated element, step, or component. Thus, the terms “include” or “including” should be interpreted to recite: “comprise, consist of, or consist essentially of.” The transition term “comprise” or “comprises” means has, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the implementation to the specified elements, steps, ingredients or components and to those that do not materially affect the implementation. As used herein, the term “based on” is equivalent to “based at least partly on,” unless otherwise specified.

Unless otherwise indicated, all numbers expressing quantities, properties, conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing implementations (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate implementations of the disclosure and does not pose a limitation on the scope of the disclosure. No language in the specification should be construed as indicating any non-claimed element essential to the practice of implementations of the disclosure.

Groupings of alternative elements or implementations disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Variants of the sequences disclosed and referenced herein are also included. Variants of the protein, nucleic acid, and gene sequences disclosed herein also include sequences with at least 70% sequence identity, 80% sequence identity, 85% sequence, 90% sequence identity, 95% sequence identity, 96% sequence identity, 97% sequence identity, 98% sequence identity, or 99% sequence identity to the protein, nucleic acid, or gene sequences disclosed herein.

“% sequence identity” refers to a relationship between two or more sequences, as determined by comparing the sequences. In the art, “identity” also means the degree of sequence relatedness between protein, nucleic acid, or gene sequences as determined by the match between strings of such sequences. “Identity” (often referred to as “similarity”) can be readily calculated by known methods, including those described in: Computational Molecular Biology (Lesk, A. M., ed.) Oxford University Press, NY (1988); Biocomputing: Informatics and Genome Projects (Smith, D. W., ed.) Academic Press, NY (1994); Computer Analysis of Sequence Data, Part I (Griffin, A. M., and Griffin, H. G., eds.) Humana Press, NJ (1994); Sequence Analysis in Molecular Biology (Von Heijne, G., ed.) Academic Press (1987); and Sequence Analysis Primer (Gribskov, M. and Devereux, J., eds.) Oxford University Press, NY (1992). Preferred methods to determine identity are designed to give the best match between the sequences tested. Methods to determine identity and similarity are codified in publicly available computer programs. Sequence alignments and percent identity calculations may be performed using the Megalign program of the LASERGENE bioinformatics computing suite (DNASTAR, Inc., Madison, Wisconsin). Multiple alignment of the sequences can also be performed using the Clustal method of alignment (Higgins and Sharp CABIOS, 5, 151-153 (1989) with default parameters (GAP PENALTY=10, GAP LENGTH PENALTY=10). Relevant programs also include the GCG suite of programs (Wisconsin Package Version 9.0, Genetics Computer Group (GCG), Madison, Wisconsin); BLASTP, BLASTN, BLASTX (Altschul et al., J. Mol. Biol. 215:403-410 (1990); DNASTAR (DNASTAR, Inc., Madison, Wisconsin); and the FASTA program incorporating the Smith-Waterman algorithm (Pearson, Comput. Methods Genome Res., [Proc. Int. Symp.](1994), Meeting Date 1992, 111-20. Editor(s): Suhai, Sandor. Publisher: Plenum, New York, N.Y. Within the context of this disclosure It will be understood that where sequence analysis software is used for analysis, the results of the analysis are based on the “default values” of the program referenced. As used herein “default values” will mean any set of values or parameters, which originally load with the software when first initialized.

Variants also include nucleic acid molecules that hybridizes under stringent hybridization conditions to a sequence disclosed herein and provide the same function as the reference sequence. Exemplary stringent hybridization conditions include an overnight incubation at 42 °C in a solution including 50% formamide, 5×SSC (750 mM NaCl, 75 mM trisodium citrate), 50 mM sodium phosphate (pH 7.6), 5Xdenhardt's solution, 10% dextran sulfate, and 20 μg/ml denatured, sheared salmon sperm DNA, followed by washing the filters in 0.1×SSC at 50 °C. Changes in the stringency of hybridization and signal detection are primarily accomplished through the manipulation of formamide concentration (lower percentages of formamide result in lowered stringency); salt conditions, or temperature. For example, moderately high stringency conditions include an overnight incubation at 37 °C in a solution including 6×SSPE (20×SSPE=3M NaCl; 0.2M NaH₂PO₄; 0.02M EDTA, pH 7.4), 0.5% SDS, 30% formamide, 100 μg/ml salmon sperm blocking DNA; followed by washes at 50 °C with 1×SSPE, 0.1% SDS. In addition, to achieve even lower stringency, washes performed following stringent hybridization can be done at higher salt concentrations (e.g. 5×SSC). Variations in the above conditions may be accomplished through the inclusion and/or substitution of alternate blocking reagents used to suppress background in hybridization experiments. Typical blocking reagents include Denhardt's reagent, BLOTTO, heparin, denatured salmon sperm DNA, and commercially available proprietary formulations. The inclusion of specific blocking reagents may require modification of the hybridization conditions described above, due to problems with compatibility.

Unless otherwise indicated, the practice of the present disclosure can employ conventional techniques of immunology, molecular biology, microbiology, cell biology and recombinant DNA. These methods are described in the following publications. See, e.g., Sambrook, et al. Molecular Cloning: A Laboratory Manual, 2^{nd} Edition (1989); F. M. Ausubel, et al. eds., Current Protocols in Molecular Biology, (1987); the series Methods IN Enzymology (Academic Press, Inc.); M. MacPherson, et al., PCR: A Practical Approach, IRL Press at Oxford University Press (1991); MacPherson et al., eds. PCR 2: Practical Approach, (1995); Harlow and Lane, eds. Antibodies, A Laboratory Manual, (1988); and R. I. Freshney, ed. Animal Cell Culture (1987). All references to printed publications, patent applications, patents, or other resources are incorporated by reference herein in their entirety.

Certain implementations are described herein, including the best mode known to the inventors for carrying out implementations of the disclosure. Of course, variations on these described implementations will become apparent to those of ordinary skill in the art upon reading the foregoing description. The inventor expects skilled artisans to employ such variations as appropriate, and the inventors intend for implementations to be practiced otherwise than specifically described herein. Accordingly, the scope of this disclosure includes all modifications and equivalents of the subject matter recited in the claims appended hereto as permitted by applicable law. Moreover, any combination of the above-described elements in all possible variations thereof is encompassed by implementations of the disclosure unless otherwise indicated herein or otherwise clearly contradicted by context.

1-74. (canceled)

75. A method of delivering an agent to a nucleus of a cell, the method comprising:
providing, to a sample or a subject comprising the cell, the agent conjugated to a nuclear targeting tag, the nuclear targeting tag comprising:
an NLS comprising the sequence set forth in SEQ ID NO: 4 or SEQ ID NO: 6; and
a polyarginine linker comprising three, four, five, six, seven, eight, nine, ten, eleven, or twelve arginine residues.

76. The method of claim 75, wherein the agent comprises at least one of a peptide, a protein, a detectable label, a therapeutic agent, a vector, a nucleic acid, or a chemical agent; and/or
wherein the cell is a brain cell, a central nervous system cell, a glutamatergic cell, a gamma-aminobutyric acid(GABA)ergic cell, a heart cell, an epithelial cell, an endothelial cell, a muscle cell, an immune cell, an adipocyte, an osteocyte, or a stem cell.

77. The method of claim 76, wherein the therapeutic agent comprises at least one of a gene therapy agent, a small molecule, a peptide, a protein, an immune therapy, a vaccine, or an enzyme;
wherein the detectable label comprises at least one of a fluorescent label, a luminescent label, a radioisotope, a contrast reagent, or a quantum dot; or
wherein the nuclear targeting tag is at a N terminus or a C terminus of the peptide or protein.

78. The method of claim 77, wherein the gene therapy agent comprises at least one of a microRNA, an antisense oligonucleotide, a short interfering RNA, a short hairpin RNA, a plasmid, a viral vector, a non-viral vector, a clustered regularly interspaced short palindromic repeats (CRISPR) RNA, or a Cas protein; and/or
wherein the fluorescent label comprises at least one of enhanced green fluorescent protein (EGFP), oScarlet, mKate2, miRFP670, AausFP1, mVenus-ME, E2-Crimson or mGreenLantern.

79. The method of claim 75, wherein the cell is a mammalian cell; and/or
wherein the cell is a mouse cell, a rat cell, a non-human primate cell, or a human cell.

80. The method of claim 75, wherein the providing comprises:
applying a fluid comprising the agent conjugated to the nuclear targeting tag;
administering the agent conjugated to the nuclear targeting tag to a living subject; or
transforming the cell with an expression construct comprising a nucleic acid sequence encoding the agent.

81. The method of claim 80, wherein the living subject is a subject in need of treating,
wherein the agent comprises a therapeutic agent, and
wherein the administering comprises administering a therapeutically effective amount of the therapeutic agent to the living subject, thereby treating the living subject in need.

82. The method of claim 75, wherein the agent comprises or is conjugated to a detectable label, the method further comprising:
detecting, in the sample, the detectable label.

83. The method of claim 82, wherein the detecting comprises generating an image depicting the sample, performing fluorescence activated cell sorting, or performing automated image segmentation.

84. The method of claim 75, wherein the nuclear targeting tag comprises the sequence set forth in SEQ ID NO: 3 or SEQ ID NO: 5.

85. An isolated agent comprising a nuclear targeting tag comprising:
an NLS comprising the sequence set forth in SEQ ID NO: 4 or SEQ ID NO: 6; and
an arginine linker comprising three, four, five, six, seven, eight, nine, ten, eleven, or twelve arginine residues.

86. The isolated agent of claim 85, wherein the isolated agent further comprises at least one of a protein, a peptide, a detectable label, a therapeutic agent, a vector, a nucleic acid, or a chemical agent; and/or
wherein the nuclear targeting tag comprises the sequence set forth in SEQ ID NO: 3 or SEQ ID NO: 5.

87. A host cell comprising the isolated agent of claim 85.

88. The host cell of claim 87, wherein the host cell expresses the isolated agent; and/or
wherein the isolated agent is targeted to a nucleolus of the host cell.

89. A host animal comprising the isolated agent of claim 85.

90. The host animal of claim 89, wherein the isolated agent is expressed, by the host animal, for at least 3 weeks.

91. The host animal of claim 90, wherein a parameter representing a normal function of the host animal is within a normal physiological range for the host after expression of the isolated agent for at least 3 weeks.

92. The host animal of claim 89, wherein the host animal is a mammal; and/or
wherein the host animal is a mouse, a rat, a non-human primate, or a human.

93. An expression construct, comprising:
a nucleic acid encoding a nuclear targeting tag comprising:
an NLS comprising the sequence set forth in SEQ ID NO: 4 or SEQ ID NO: 6; and
an arginine linker comprising three, four, five, six, seven, eight, nine, ten, eleven, or twelve arginine residues,
the nucleic acid being adjacent to a multicloning site.

94. The expression construct of claim 93, wherein the nuclear targeting tag comprises the sequence set forth in SEQ ID NO: 3 or SEQ ID NO: 5.
